(19) United States
(12) Patent Application Publication
(10) Pub. No.: US 2023/0313252 A1
(43) Pub. Date: Oct. 5, 2023

(54) CELLULAR PRODUCTION OF SIALYLATED DI AND/OR OLIGOSACCHARIDES

(71) Applicant: INBIOSE N.V., Zwijnaarde (BE)

(72) Inventors: Sofie Aesaert, Zwijnaarde (BE); Joeri Beauprez, Zwijnaarde (BE); Pieter Coussement, Zwijnaarde (BE); Thomas Decoene, Zwijnaarde (BE); Nausicaä Lannoo, Zwijnaarde (BE); Gert Peters, Zwijnaarde (BE); Kristof Vandewalle, Zwijnaarde (BE)

(21) Appl. No.: 18/041,137

(22) PCT Filed: Aug. 10, 2021
(86) PCT No.: PCT/EP2021/072274

§ 371 Date: Feb. 9, 2023

(30) Foreign Application Priority Data
Aug. 10, 2020 (EP) 20190198.0
Aug. 10, 2020 (EP) 20190200.4
Aug. 10, 2020 (EP) 20190201.2
Aug. 10, 2020 (EP) 20190202.0
Aug. 10, 2020 (EP) 20190203.8
Aug. 10, 2020 (EP) 20190205.3
Apr. 16, 2021 (EP) 21168997.1

(51) Int. Cl. C12P 19/18

(52) U.S. Cl. CPC C12P 19/18

(57) ABSTRACT

The disclosure is in the technical field of synthetic biology and metabolic engineering. More particularly, the disclosure is in the technical field of metabolically engineered cells and use of the cells in a cultivation or fermentation. The disclosure describes a metabolically engineered cell and a method by cultivation or fermentation with the cell for production of a sialylated di- and/or oligosaccharide. The metabolically engineered cell comprises a pathway for production of the sialylated di- and/or oligosaccharide and is modified for expression and/or overexpression of multiple coding DNA sequences encoding one or more isoproteins that catalyze the same chemical reaction. Furthermore, the disclosure provides for purification of the sialylated di- and/or oligosaccharide from the cultivation.

### CROSS-REFERENCE TO RELATED APPLICATIONS

This application is a national phase entry under 35 U.S.C. § 371 of International Patent Application PCT/EP2021/072274, filed Aug. 10, 2021, designating the United States of America and published as International Patent Publication WO 2022/034080 A1 on Feb. 17, 2022, which claims the benefit under Article 8 of the Patent Cooperation Treaty to European Patent Application Serial No. 20190198.0, filed Aug. 10, 2020, European Patent Application Serial No. 20190200.4, filed Aug. 10, 2020, European Patent Application Serial No. 20190201.2, filed Aug. 10, 2020, European Patent Application Serial No. 20190202.0, filed Aug. 10, 2020, European Patent Application Serial No. 20190203.8, filed Aug. 10, 2020, European Patent Application Serial No. 20190205.3, filed Aug. 10, 2020, and European Patent Application Serial No. 21168997.1, filed Apr. 16, 2021.

### STATEMENT REGARDING ELECTRONIC FILING OF A SEQUENCE LISTING

Pursuant to 37 C.F.R. § 1.821, a Sequence Listing ASCII text file entitled “4006-P17245US_041-PCT-US_SequenceListing_ST25.txt,” 207,736 bytes in size, generated Feb. 7, 2023, has been submitted via EFS-Web is provided in lieu of a paper copy. This Sequence Listing is hereby incorporated by reference into the specification for its disclosures.

### TECHNICAL FIELD

This disclosure is in the technical field of synthetic biology and metabolic engineering. More particularly, the disclosure is in the technical field of metabolically engineered cells and use of the cells in a cultivation or fermentation. The disclosure describes a metabolically engineered cell and a method by cultivation or fermentation with the cell for production of a sialylated di- and/or oligosaccharide. The metabolically engineered cell comprises a pathway for production of the sialylated di- and/or oligosaccharide and is modified for expression and/or overexpression of multiple coding DNA sequences encoding one or more isoproteins that catalyze the same chemical reaction. Furthermore, the disclosure provides for purification of the sialylated di- and/or oligosaccharide from the cultivation.

### BACKGROUND

Sialylated di- and oligosaccharides, often present as glyco-conjugated forms to proteins and lipids, are involved in many vital phenomena such as development, differentiation, fertilization, embryogenesis, host pathogen adhesion and inflammation. Sialylated oligosaccharides can also be present as unconjugated glycans in body fluids and mammalian milk wherein they modulate as bioactive glycans in important developmental and immunological processes (Bode, Early Hum. Dev. 2015, 91(11): 619-622; Bode, Nestle Nutr. Inst. Workshop Ser. 2019, 90: 191-201; Reily et al., Nat. Rev. Nephrol. 2019, 15: 346-366; Varki, Glycobiology 2017, 27: 3-49; Walsh et al., J. Funct. Foods 2020, 72: 10474). There is large scientific and commercial interest in sialylated di- and oligosaccharides due to their wide functional spectrum. Yet, the availability of sialylated di- and/or oligosaccharides is limited as production relies on chemical or chemo-enzymatic synthesis or on purification from natural sources such as e.g., animal milk. Chemical synthesis methods are laborious and time-consuming and because of the large number of steps involved they are difficult to scale-up. Enzymatic approaches using nucleotide-activated sugars and glycosyltransferases offer many advantages above chemical synthesis. Glycosyltransferases catalyze the transfer of a sugar moiety from a nucleotide-activated sugar donor onto saccharide or non-saccharide acceptors (Coutinho et al., J. Mol. Biol. 2003, 328: 307-317). These glycosyltransferases are the source for biotechnologists to synthesize sialylated di- and oligosaccharides and are used both in (chemo)enzymatic approaches as well as in cell-based production systems. However, stereospecificity and regioselectivity of glycosyltransferases are still a formidable challenge. In addition, chemo-enzymatic approaches need to regenerate in situ nucleotide-activated sugar donors. Cellular production of sialylated di- and oligosaccharides needs tight control of spatiotemporal availability of adequate levels of nucleotide-activated sugar donors in proximity of complementary glycosyltransferases. Due to these difficulties, current methods often result in small-scale synthesis of sialylated di- and/or oligosaccharides.

PEP or phosphoenolpyruvate is a common precursor in the anabolism of a cell and of key importance for the synthesis of secondary metabolites such as flavonoids, aromatic amino acids and many monosaccharide subunits of sialylated di- and oligosaccharides or sialylated di- and oligosaccharide modifications. Such monosaccharide subunits are, for instance, N-acetylneuraminic acid, legionaminic acid, ketodeoxyoctonate, keto-deoxy-nonulonic acid, pseudaminic acid, N, N′-diacetyl-8-epilegionaminate, N-acetyl-D-muramate and their nucleotide and phosphorylated derivatives. To enhance synthesis of these monosaccharide subunits and sialylated di- and/or oligosaccharides, the PEP concentration in the cell can be enhanced by means of overexpression and deletion of several genes.

Zhu et al. (Biotechnol. Lett. 2017, 39: 227-234) has shown that by the overexpression of PEP synthase (EC: 2.7.9.2) and PEP carboxykinase (EC: 4.1.1.49) the synthesis of N-acetylneuraminic acid was increased by 96.4% and 61% compared to the control respectively, combined overexpression increased the synthesis further up to 116.7% compared to the control. Zhu et al. (Biotechnol. Lett 2016, doi 10.1007/s10529-016-2215-z) has further shown that the deletion of a substrate phosphotransferase (PTS) system like the N-acetylglucosamine PTS system encoded by the gene nagE in E. coli, transporting and phosphorylating with the use of PEP N-acetylglucosamine (GlcNAc) and glucosamine (GlcN) into the cell, or like the mannose PTS system encoded by the genes manX, manY and manZ in E. coli, transporting and phosphorylating with the use of PEP mannose, N-acetylmannosamine, glucose, fructose, GlcN and GlcNAc into the cell, increases Neu5Ac synthesis significantly. The upregulation of ppsA in E. coli was later also shown to be effective in EP3697805 and EP3575404, combining also ppsA overexpression with the deletion of manXYZ and nagE.

Zhang et al. (Biotech and Bioeng. 2018, 115(9): 217-2231) improved PEP synthesis in Bacillus subtilis in a similar fashion. The glucose PTS system was deleted to reduce PEP usages upon glucose uptake, the gene pyruvate kinase (EC: 2.7.1.40) was deleted to reduce PEP consumption and the gene PEP carboxykinase (EC: 4.1.1.49) was overexpressed to enhance the flux toward. To compensate for the deletion of the glucose PTS system, glucose permease and glucokinase were used to internalize and phosphorylate glucose in the cell. Further, the malic enzyme (EC: 1.1.1.38, EC: 1.1.1.39 or EC: 1.1.1.40) was introduced to increase the flux from the Krebs cycle toward pyruvate, the precursor of PEP. A reduced glycolysis and the introduction of the Entner-Doudoroff pathway further enhanced the production of N-acetylneuraminate. Note that these strains are in their basis modified in their acetate and lactate synthesis capacity, which inherently leads to improved availability of PEP, pyruvate and acetyl-CoA.

Zhang et al. (Biotech. Adv. 2019, 37: 787-800) also reviewed and described how the precursors of N-acetylneuraminic acid and sialylated oligosaccharides can be modulated. By impacting the PEP and pyruvate availability in the cell, the flux toward sialylated oligosaccharides and N-acetylneuraminate (or other monosaccharide subunits as described above) is enhanced. Also here, techniques are described to delete or knock down the glycolysis pathway (comprising phosphofructokinase (pfkA gene, E.C.:2.7.1.11) and pyruvate kinase (pyk, EC: 2.7.1.40)) and to upregulate the phosphoenolpyruvate synthase gene (ppsA, EC: 2.7.9.2). Introduction or overexpression of the Entner-Doudoroff pathway and reduced PTS activity further led to improvements in synthesis. The system described was not only achieved by overexpression or deletions, but also by dynamic control through biosensors, which selectively upregulate and downregulate reactions in the cellular biochemistry.

### SUMMARY OF THE DISCLOSURE

Provided are tools and methods by means of which a sialylated di- and/or oligosaccharide can be produced by a cell and preferably in an efficient, time and cost-effective way and which yields high amounts of the desired sialylated di- and/or oligosaccharide.

Provided are a cell and a method for the production of a sialylated di- and/or oligosaccharide wherein the cell is metabolically engineered with a pathway for the production of the sialylated di- and/or oligosaccharide and wherein the cell is modified with multiple coding DNA sequences that are capable to express and/or overexpress one or more proteins that catalyze the same chemical reaction. Surprisingly, it has now been found that the cell of disclosure which is metabolically engineered for the production of a sialylated di- and/or oligosaccharide does not suffer from clonal instability, clonal heterogeneity or transgene silencing by the introduction of multiple coding DNA sequences that encode one or more proteins that catalyze the same chemical reaction. The introduction and expression and/or overexpression of the multiple coding DNA sequences in the cell of disclosure preferably has a positive effect on fermentative production of the sialylated di- and/or oligosaccharide, and even more preferably, provide a better yield, productivity, specific productivity and/or growth speed of the cell when used to metabolically engineer a cell producing the sialylated di- and/or oligosaccharide when compared to a cell with the same genetic background but lacking the multiple coding DNA sequences as defined in the disclosure. The disclosure also provides a method for the production of a sialylated di- and/or oligosaccharide. The method comprises the steps of providing a cell comprising a pathway for the production of a sialylated di- and/or oligosaccharide, wherein the cell is modified with multiple coding DNA sequences encoding one or more proteins that catalyze the same chemical reaction and cultivating the cell under conditions permissive to produce the sialylated di- and/or oligosaccharide. The proteins encoded by the multiple coding DNA sequences comprise, amongst others, enzymes involved in the synthesis of a nucleotide-activated sugar, wherein the nucleotide-activated sugar is to be used in the production of the sialylated di- and/or oligosaccharide, and membrane transporter proteins. The disclosure also provides methods to separate the sialylated di- and/or oligosaccharide.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the disclosure disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims which follow, reference characters used to designate claim steps are provided for convenience of description only, and are not intended to imply any particular order for performing the steps.

In this document and in its claims, the verb “to comprise” and its conjugations is used in its non-limiting sense to mean that items following the word are included, but items not specifically mentioned are not excluded. Throughout the disclosure, the verb “to comprise” may be replaced by “to consist” or “to consist essentially of” and vice versa. In addition the verb “to consist” may be replaced by “to consist essentially of” meaning that a composition as defined herein may comprise additional component(s) than the ones specifically identified, the additional component(s) not altering the unique characteristic of the disclosure. In addition, reference to an element by the indefinite article “a” or “an” does not exclude the possibility that more than one of the elements is present, unless the context clearly requires that there is one and only one of the elements. The indefinite article “a” or “an” thus usually means “at least one.” Throughout the disclosure, unless explicitly stated otherwise, the articles “a” and “an” are preferably replaced by “at least two,” more preferably by “at least three,” even more preferably by “at least four,” even more preferably by “at least five,” even more preferably by “at least six,” most preferably by “at least seven.”

Throughout the disclosure, unless explicitly stated otherwise, the features “synthesize,” “synthesized” and “synthesis” are interchangeably used with the features “produce,” “produced” and “production,” respectively.

Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference. The full content of the priority applications, including EP21168997, EP20190198, EP20190200, EP20190205 are also incorporated by reference to the same extent as if the priority applications were specifically and individually indicated to be incorporated by reference.

According to the disclosure, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the disclosure. Moreover, DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated.

The term “endogenous,” within the context of the disclosure refers to any polynucleotide, polypeptide or protein sequence which is a natural part of a cell and is occurring at its natural location in the cell chromosome and of which the control of expression has not been altered compared to the natural control mechanism acting on its expression. The term “exogenous” refers to any polynucleotide, polypeptide or protein sequence which originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “polynucleotide encoding a polypeptide” as used herein encompasses polynucleotides that include a sequence encoding a polypeptide of the disclosure. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

The term “modified expression” of a gene relates to a change in expression compared to the wild type expression of the gene in any phase of the production process of the desired sialylated di- and/or oligosaccharide. The modified expression is either a lower or higher expression compared to the wild type, wherein the term “higher expression” is also defined as “overexpression” of the gene in the case of an endogenous gene or “expression” in the case of a heterologous gene that is not present in the wild type strain. Lower expression is obtained by means of common well-known technologies for a skilled person (such as the usage of siRNA, CrispR, CrispRi, riboswitches, recombineering, homologous recombination, ssDNA mutagenesis, RNAi, miRNA, asRNA, mutating genes, knocking-out genes, transposon mutagenesis, . . . ) which are used to change the genes in such a way that they are less-able (i.e., statistically significantly ‘less-able’ compared to a functional wild-type gene) or completely unable (such as knocked-out genes) to produce functional final products. The term “riboswitch” as used herein is defined to be part of the messenger RNA that folds into intricate structures that block expression by interfering with translation. Binding of an effector molecule induces conformational change(s) permitting regulated expression post-transcriptionally. Overexpression or expression is obtained by means of common well-known technologies for a skilled person (such as the usage of artificial transcription factors, de novo design of a promoter sequence, ribosome engineering, introduction or re-introduction of an expression module at euchromatin, usage of high-copy-number plasmids), wherein the gene is part of an “expression cassette,” which relates to any sequence in which a promoter sequence, untranslated region sequence (containing either a ribosome binding sequence or Kozak sequence), a coding sequence (for instance, an N-acylneuraminate cytidylyltransferase) and optionally a transcription terminator is present, and leading to the expression of a functional active protein. The expression is either constitutive or conditional or regulated or tuneable.

The term “constitutive expression” is defined as expression that is not regulated by transcription factors other than the subunits of RNA polymerase (e.g., the bacterial sigma factors) under certain growth conditions. Non-limiting examples of such transcription factors are CRP, LacI, ArcA, Cra, IclR in E. coli, or, Aft2p, Crz1p, Skn7 in Saccharomyces cerevisiae, or, DeoR, GntR, Fur in B. subtilis. These transcription factors bind on a specific sequence and may block or enhance expression in certain growth conditions. The RNA polymerase is the catalytic machinery for the synthesis of RNA from a DNA template. RNA polymerase binds a specific sequence to initiate transcription, for instance, via a sigma factor in prokaryotic hosts or via MTF1 in yeasts. Constitutive expression offers a constant level of expression with no need for induction or repression.

The term “regulated expression” is defined as expression that is regulated by transcription factors other than the subunits of RNA polymerase (e.g., bacterial sigma factors like s⁷⁰, s⁵⁴, or related s-factors and the yeast mitochondrial RNA polymerase specificity factor MTF1 that co-associate with the RNA polymerase core enzyme) under certain growth conditions. Examples of such transcription factors are described above. Commonly expression regulation is obtained by means of an inducer, such as but not limited to IPTG, arabinose, rhamnose, fucose, allo-lactose or pH shifts, or temperature shifts or carbon depletion or substrates or the produced product.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

The term “wild type” refers to the commonly known genetic or phenotypical situation as it occurs in nature.

The term “modified expression or activity of a protein” as used herein refers to i) higher expression or overexpression of an endogenous protein, ii) expression of a heterologous protein or iii) expression and/or overexpression of a variant protein that has a higher activity compared to the wild-type (i.e., native) protein.

As used herein, the term “mammary cell(s)” generally refers to mammary epithelial cell(s), mammary-epithelial luminal cell(s), or mammalian epithelial alveolar cell(s), or any combination thereof. As used herein, the term “mammary-like cell(s)” generally refers to cell(s) having a phenotype/genotype similar (or substantially similar) to natural mammary cell(s) but is/are derived from non-mammary cell source(s). Such mammary-like cell(s) may be engineered to remove at least one undesired genetic component and/or to include at least one predetermined genetic construct that is typical of a mammary cell. Non-limiting examples of mammary-like cell(s) may include mammary epithelial-like cell(s), mammary epithelial luminal-like cell(s), non-mammary cell(s) that exhibits one or more characteristics of a cell of a mammary cell lineage, or any combination thereof. Further non-limiting examples of mammary-like cell(s) may include cell(s) having a phenotype similar (or substantially similar) to natural mammary cell(s), or more particularly a phenotype similar (or substantially similar) to natural mammary epithelial cell(s). A cell with a phenotype or that exhibits at least one characteristic similar to (or substantially similar to) a natural mammary cell or a mammary epithelial cell may comprise a cell (e.g., derived from a mammary cell lineage or a non-mammary cell lineage) that exhibits either naturally, or has been engineered to, be capable of expressing at least one milk component.

As used herein, the term “non-mammary cell(s)” may generally include any cell of non-mammary lineage. In the context of the disclosure, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

Throughout the disclosure, unless explicitly stated otherwise, the expressions “capable of . . . <verb>” and “capable to . . . <verb>” are preferably replaced with the active voice of the verb and vice versa. For example, the expression “capable of expressing” is preferably replaced with “expresses” and vice versa, i.e., “expresses” is preferably replaced with “capable of expressing.”

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the present disclosure contemplates making functional variants by modifying the structure of a protein of interest as used in the disclosure. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide.

The term “functional homolog” as used herein describes those molecules that have sequence similarity and also share at least one functional characteristic such as a biochemical activity. More specifically, the term “functional homolog” as used herein describes those proteins that have sequence similarity (in other words, homology) and at the same time have at least one functional similarity such as a biochemical activity (Altenhoff et al., PLoS Comput. Biol. 8 (2012) e1002514).

Functional homologs are sometimes referred to as orthologs, where “ortholog” refers to a homologous gene or protein that is the functional equivalent of the referenced gene or protein in another species. Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events.

Functional homologs can be identified by analysis of nucleotide and polypeptide sequence alignments. For example, performing a query on a database of nucleotide or polypeptide sequences can identify homologs of e.g., a biomass-modulating polypeptide, a glycosyltransferase, a protein involved in nucleotide-activated sugar synthesis or a membrane transporter protein. Sequence analysis can involve BLAST, Reciprocal BLAST, or PSI-BLAST analysis of non-redundant databases using amino acid sequence of a biomass-modulating polypeptide, a glycosyltransferase, a protein involved in nucleotide-activated sugar synthesis or a membrane transporter protein, respectively, as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide, a glycosyltransferase, a protein involved in nucleotide-activated sugar synthesis or a membrane transporter protein, respectively. Amino acid sequence similarity allows for conservative amino acid substitutions, such as substitution of one hydrophobic residue for another or substitution of one polar residue for another. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out in order to narrow the number of candidates to be further evaluated. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

A domain can be characterized, for example, by a Pfam (El-Gebali et al., Nucleic Acids Res. 47 (2019) D427-D432), an IPR (InterPro domain) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (www.ncbi.nlm.nih.gov/cdd) (Lu et al., Nucleic Acids Res. 48 (2020) D265-D268), a PTHR domain (www.pantherdb.org) (Mi et al., Nucleic Acids. Res. 41 (2013) D377-D386; Thomas et al., Genome Research 13 (2003) 2129-2141) or a PATRIC identifier or PATRIC DB global family domain (www.patricbrc.org/) (Davis et al., Nucleic Acids Res. 48(D1) (2020) D606-D612). It should be understood for those skilled in the art that for the databases used herein, comprising Pfam 32.0 (released September 2018), CDD v3.17 (released 3 Apr. 2019), eggnogdb 4.5.1 (released September 2016), InterPro 75.0 (released 4 Jul. 2019), TCDB (released 17 Jun. 2019) and PATRIC 3.6.9 (released March 2020), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

Throughout the disclosure, the sequence of a polynucleotide can be represented by a SEQ ID NO or alternatively by a GenBank NO. Therefore, the terms “polynucleotide SEQ ID NO” and “polynucleotide GenBank NO.” can be interchangeably used, unless explicitly stated otherwise.

Throughout the disclosure, the sequence of a polypeptide can be represented by a SEQ ID NO or alternatively by an UniProt ID or GenBank NO. Therefore, the terms “polypeptide SEQ ID NO” and “polypeptide UniProt ID” and “polypeptide GenBank NO.” can be interchangeably used, unless explicitly stated otherwise.

The term “isoproteins” as used herein refers to any family of closely related enzymes or proteins that have similar structural and functional properties, catalysing the same chemical reaction.

The term “chemical reaction” as used herein refers to a process in which one or more substances, the reactants, are converted or translocated to one or more different substances or locations, the products or cellular location, respectively. Substances are either chemical elements or compounds. A chemical reaction rearranges or transports the constituent atoms or molecules of the reactants to create different substances as products or translocates the atoms or molecules to either the cytoplasm or the extracellular space. Examples of such chemical reactions are biochemical, enzymatic, organic chemical, inorganic chemical, biocatalytic and metabolic reactions or are transport reactions comprising import, efflux, secretion and excretion reactions.

The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity may be calculated globally over the full-length sequence of the reference sequence, resulting in a global percent identity score. Alternatively, percent identity may be calculated over a partial sequence of the reference sequence, resulting in a local percent identity score. Using the full-length of the reference sequence in a local sequence alignment results in a global percent identity score between the test and the reference sequence.

Percent identity can be determined using different algorithms like, for example, BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle.

The BLAST (Basic Local Alignment Search Tool)) method of alignment is an algorithm provided by the National Center for Biotechnology Information (NCBI) to compare sequences using default parameters. The program compares nucleotide or protein sequences to sequence databases and calculates the statistical significance. PSI-BLAST (Position-Specific Iterative Basic Local Alignment Search Tool) derives a position-specific scoring matrix (PSSM) or profile from the multiple sequence alignment of sequences detected above a given score threshold using protein-protein BLAST (BLASTp). The BLAST method can be used for pairwise or multiple sequence alignments. Pairwise Sequence Alignment is used to identify regions of similarity that may indicate functional, structural and/or evolutionary relationships between two biological sequences (protein or nucleic acid). The web interface for BLAST is available at: blast.ncbi.nlm.nih.gov/Blast.cgi.

Clustal Omega (Clustal W) is a multiple sequence alignment program that uses seeded guide trees and HMM profile-profile techniques to generate alignments between three or more sequences. It produces biologically meaningful multiple sequence alignments of divergent sequences. The web interface for Clustal W is available at www.ebi.ac.uk/Tools/msa/clustalo/. Default parameters for multiple sequence alignments and calculation of percent identity of protein sequences using the Clustal W method are: enabling de-alignment of input sequences: FALSE; enabling mbed-like clustering guide-tree: TRUE; enabling mbed-like clustering iteration: TRUE; Number of (combined guide-tree/HMM) iterations: default(0); Max Guide Tree Iterations: default [−1]; Max HMM Iterations: default [−1]; order: aligned.

MatGAT (Matrix Global Alignment Tool) is a computer application that generates similarity/identity matrices for DNA or protein sequences without needing pre-alignment of the data. The program performs a series of pairwise alignments using the Myers and Miller global alignment algorithm, calculates similarity and identity, and then places the results in a distance matrix. The user may specify which type of alignment matrix (e.g., BLOSUM50, BLOSUM62, and PAM250) to employ with their protein sequence examination.

EMBOSS Needle (galaxy-iuc.github.io/emboss-5.0-docs/needle.html) uses the Needleman-Wunsch global alignment algorithm to find the optimal alignment (including gaps) of two sequences when considering their entire length. The optimal alignment is ensured by dynamic programming methods by exploring all possible alignments and choosing the best. The Needleman-Wunsch algorithm is a member of the class of algorithms that can calculate the best score and alignment in the order of mn steps, (where ‘n’ and ‘m’ are the lengths of the two sequences). The gap open penalty (default 10.0) is the score taken away when a gap is created. The default value assumes you are using the EBLOSUM62 matrix for protein sequences. The gap extension (default 0.5) penalty is added to the standard gap penalty for each base or residue in the gap. This is how long gaps are penalized.

As used herein, a polypeptide having an amino acid sequence having at least 80% sequence identity to the full-length sequence of a reference polypeptide sequence is to be understood as that the sequence has 80%, 81%, 82%, 83%, 84%, 85%, 86%, 87%, 88%, 89%, 90%, 91%, 91.50%, 92.00%, 92.50%, 93.00%, 93.50%, 94.00%, 94.50% 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of the amino acid sequence of the reference polypeptide sequence. Throughout the disclosure, unless explicitly specified otherwise, a polypeptide (or DNA sequence) comprising/consisting/having an amino acid sequence (or nucleotide sequence) having at least 80% sequence identity to the full-length amino acid sequence (or nucleotide sequence) of a reference polypeptide (or nucleotide sequence), usually indicated with a SEQ ID NO or UniProt ID or Genbank NO., preferably has at least 85%, 90%, 91%, 92%, 93%, 94%, 95%, 96%, 97%, 98% or 99%, more preferably has at least 85%, even more preferably has at least 90%, most preferably has at least 95%, sequence identity to the full length reference sequence.

For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM65. In a preferred embodiment, sequence identity is calculated based on the full-length sequence of a given SEQ ID NO, i.e., the reference sequence, or a part thereof. Part thereof preferably means at least 50%, 60%, 70%, 80%, 90% or 95% of the complete reference sequence.

The terms “sialic acid,” “N-acetylneuraminate,” “N-acylneuraminate,” “N-acetylneuraminic acid” are used interchangeably and refer to an acidic sugar with a nine-carbon backbone comprising but not limited to Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4, Neu4,5,7,8,9Ac5 and Neu5Gc.

Neu4Ac is also known as 4-O-acetyl-5-amino-3,5-dideoxy-D-glycero-D-galacto-non-2-ulopyranosonic acid or 4-O-acetyl neuraminic acid and has C₁₁H₁₉NO₉ as molecular formula. Neu5Ac is also known as 5-acetamido-3,5-dideoxy-D-glycero-D-galacto-non-2-ulopyranosonic acid, D-glycero-5-acetamido-3,5-dideoxy-D-galacto-non-2-ulo-pyranosonic acid, 5-(acetylamino)-3,5-dideoxy-D-glycero-D-galacto-2-nonulopyranosonic acid, 5-(acetylamino)-3,5-dideoxy-D-glycero-D-galacto-2-nonulosonic acid, 5-(acetylamino)-3,5-dideoxy-D-glycero-D-galacto-non-2-nonulosonic acid or 5-(acetylamino)-3,5-dideoxy-D-glycero-D-galacto-non-2-ulopyranosonic acid and has C₁₁H₁₉NO₉ as molecular formula. Neu4,5Ac2 is also known as N-acetyl-4-O-acetylneuraminic acid, 4-O-acetyl-N-acetylneuraminic acid, 4-O-acetyl-N-acetylneuraminate, 4-acetate 5-acetamido-3,5-dideoxy-D-glycero-D-galacto-nonulosonate, 4-acetate 5-(acetylamino)-3,5-dideoxy-D-glycero-D-galacto-2-nonulosonate, 4-acetate 5-acetamido-3,5-dideoxy-D-glycero-D-galacto-nonulosonic acid or 4-acetate 5-(acetylamino)-3,5-dideoxy-D-glycero-D-galacto-2-nonulosonic acid and has C₁₃H₂₁NO₁₀ as molecular formula. Neu5,7Ac2 is also known as 7-O-acetyl-N-acetylneuraminic acid, N-acetyl-7-O-acetylneuraminic acid, 7-O-acetyl-N-acetylneuraminate, 7-acetate 5-acetamido-3,5-dideoxy-D-glycero-D-galacto-nonulosonate, 7-acetate 5-(acetylamino)-3,5-dideoxy-D-glycero-D-galacto-2-nonulosonate, 7-acetate 5-acetamido-3,5-dideoxy-D-glycero-D-galacto-nonulosonic acid or 7-acetate 5-(acetylamino)-3,5-dideoxy-D-glycero-D-galacto-2-nonulosonic acid and has C₁₃H₂₁NO₁₀ as molecular formula. Neu5,8Ac2 is also known as 5-n-acetyl-8-o-acetyl neuraminic acid and has C₁₃H₂₁NO₁₀ as molecular formula. Neu5,9Ac2 is also known as N-acetyl-9-O-acetylneuraminic acid, 9-anana, 9-O-acetylsialic acid, 9-O-acetyl-N-acetylneuraminic acid, 5-n-acetyl-9-O-acetyl neuraminic acid, N,9-O-diacetylneuraminate or N,9-O-diacetylneuraminate and has C₁₃H₂₁NO₁₀ as molecular formula. Neu4,5,9Ac3 is also known as 5-N-acetyl-4,9-di-O-acetylneuraminic acid. Neu5,7,9Ac3 is also known as 5-N-acetyl-7,9-di-O-acetylneuraminic acid. Neu5,8,9Ac3 is also known as 5-N-acetyl-8,9-di-O-acetylneuraminic acid. Neu4,5,7,9Ac4 is also known as 5-N-acetyl-4,7,9-tri-O-acetylneuraminic acid. Neu5,7,8,9Ac4 is also known as 5-N-acetyl-7,8,9-tri-O-acetylneuraminic acid. Neu4,5,7,8,9Ac5 is also known as 5-N-acetyl-4,7,8,9-tetra-O-acetylneuraminic acid. Neu5Gc is also known as N-glycolyl-neuraminic acid, N-glycolylneuraminic acid, N-glycolylneuraminate, N-glycoloyl-neuraminate, N-glycoloylneuraminic acid, N-glycoloylneuraminic acid, 3,5-dideoxy-5-((hydroxyacetyl)amino)-D-glycero-D-galacto-2-nonulosonic acid, 3,5-dideoxy-5-(glycoloylamino)-D-glycero-D-galacto-2-nonulopyranosonic acid, 3,5-dideoxy-5-(glycoloylamino)-D-glycero-D-galacto-non-2-ulopyranosonic acid, 3,5-dideoxy-5-[(hydroxyacetyl)amino]-D-glycero-D-galacto-non-2-ulopyranosonic acid, D-glycero-5-glycolylamido-3,5-dideoxy-D-galacto-non-2-ulo-pyranosonic acid and has C₁₁H₁₉NO₁₀ as molecular formula.

The terms “NeunAc synthase,” “N-acetylneuraminic acid synthase,” “N-acetylneuraminate synthase,” “sialic acid synthase,” “NeuAc synthase,” “NeuB,” “NeuB1,” “NeuNAc synthase,” “NANA condensing enzyme,” “N-acetylneuraminate lyase synthase,” “N-acetylneuraminic acid condensing enzyme” as used herein are used interchangeably and refer to an enzyme capable to synthesize sialic acid from N-acetylmannosamine (ManNAc) in a reaction using phosphoenolpyruvate (PEP).

The terms “CMP-sialic acid synthase,” “N-acylneuraminate cytidylyltransferase,” “CMP-sialate synthase,” “CMP-NeuAc synthase,” “NeuA” and “CMP-N-acetylneuraminic acid synthase” as used herein are used interchangeably and refer to an enzyme capable to synthesize CMP-N-acetylneuraminate from N-acetylneuraminate using CTP in the reaction.

The terms “N-acylneuraminate-9-phosphate synthetase,” “NANA synthase,” “NANAS,” “NANS,” “NmeNANAS,” “N-acetylneuraminate pyruvate-lyase (pyruvate-phosphorylating)” as used herein are used interchangeably and refer to an enzyme capable to synthesize N-acylneuraminate-9-phosphate from N-acetylmannosamine-6-phosphate (ManNAc-6-phosphate) in a reaction using phosphoenolpyruvate (PEP).

The term “N-acylneuraminate-9-phosphatase” refers to an enzyme capable to dephosphorylate N-acylneuraminate-9-phosphate to synthesise N-acylneuraminate.

An N-acylglucosamine 2-epimerase is an enzyme that catalyses the reaction N-acyl-D-glucosamine=N-acyl-D-mannosamine. Alternative names for this enzyme comprise N-acetylglucosamine 2-epimerase, N-acetyl-D-glucosamine 2-epimerase, GlcNAc 2-epimerase and N-acyl-D-glucosamine 2-epimerase.

An UDP-N-acetylglucosamine 2-epimerase is an enzyme that catalyses the reaction N-acetyl-D-glucosamine=N-acetylmannosamine. Alternative names for this enzyme comprise UDP-N-acylglucosamine 2-epimerase, UDP-GlcNAc-2-epimerase and UDP-N-acetyl-D-glucosamine 2-epimerase.

An N-acetylmannosamine-6-phosphate 2-epimerase is an enzyme that catalyses the reaction N-acetyl-D-glucosamine 6-phosphate=N-acetyl-D-mannosamine 6-phosphate.

A bifunctional UDP-GlcNAc 2-epimerase/kinase is a bifunctional enzyme that catalyses the reaction UDP-N-acetyl-D-glucosamine=N-acetyl-D-mannosamine and the reaction N-acetyl-D-mannosamine+ATP=ADP+N-acetyl-D-mannosamine 6-phosphate.

The terms “N-acetylneuraminate lyase,” “Neu5Ac lyase,” “N-acetylneuraminate pyruvate-lyase,” “N-acetylneuraminic acid aldolase,” “NALase,” “sialate lyase,” “sialic acid aldolase,” “sialic acid lyase” and “nanA” are used interchangeably and refer to an enzyme that degrades N-acetylneuraminate into N-acetylmannosamine (ManNAc) and pyruvate.

The terms “N-acetylneuraminate kinase,” “ManNAc kinase,” “N-acetyl-D-mannosamine kinase” and “nanK” are used interchangeably and refer to an enzyme that phosphorylates ManNAc to synthesize N-acetylmannosamine-phosphate (ManNAc-6-P).

The terms “ManNAc-6-P isomerase,” “ManNAc-6-P 2-epimerase” and “nanE” are used interchangeably and refer to an enzyme that converts ManNAc-6-P to N-acetylglucosamine-6-phosphate (GlcNAc-6-P).

The terms “N-acetylglucosamine-6-P deacetylase” and “nagA” are used interchangeably and refer to an enzyme that catalyses the hydrolysis of the N-acetyl group of N-acetylglucosamine-6-phosphate (GlcNAc-6-P) to yield glucosamine-6-phosphate (GlcN6P) and acetate.

The terms “glucosamine-6-P deaminase,” “GlcN6P deaminase,” “glucosamine-6-phosphate isomerase,” “glmD” and “nagB” are used interchangeably and refer to an enzyme that catalyses the reversible isomerization-deamination of glucosamine-6-phosphate (GlcN6P) to form fructose-6-phosphate and an ammonium ion.

The term “glycosyltransferase” as used herein refers to an enzyme capable to catalyse the transfer of sugar moieties from activated donor molecules to specific acceptor molecules, forming glycosidic bonds. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org).

As used herein the glycosyltransferase can be selected from the list comprising but not limited to: fucosyltransferases (e.g., alpha-1,2-fucosyltransferases, alpha-1,3/1,4-fucosyltransferases, alpha-1,6-fucosyltransferases), sialyltransferases (e.g., alpha-2,3-sialyltransferases, alpha-2,6-sialyltransferases, alpha-2,8-sialyltransferases), galactosyltransferases (e.g beta-1,3-galactosyltransferases, beta-1,4-galactosyltransferases, alpha-1,3-galactosyltransferases, alpha-1,4-galactosyltransferases), glucosyltransferases, mannosyltransferases, N-acetylglucosaminyltransferases (e.g., beta-1,3-N-acetylglucosaminyltransferases, beta-1,6-N-acetylglucosaminyltransferases), N-acetylgalactosaminyltransferases (e.g., alpha-1,3-N-acetylgalactosaminyltransferases, beta-1,3-N-acetylgalactosaminyltransferases), N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

Fucosyltransferases are glycosyltransferases that transfer a fucose residue (Fuc) from a GDP-fucose (GDP-Fuc) donor onto a glycan acceptor. Fucosyltransferases comprise alpha-1,2-fucosyltransferases, alpha-1,3-fucosyltransferases, alpha-1,4-fucosyltransferases and alpha-1,6-fucosyltransferases that catalyse the transfer of a Fuc residue from GDP-Fuc onto a glycan acceptor via alpha-glycosidic bonds. Fucosyltransferases can be found but are not limited to the GT10, GT 11, GT23, GT65 and GT68 CAZy families. Sialyltransferases are glycosyltransferases that transfer a sialic acid (like Neu5Ac or Neu5Gc) from a donor (like CMP-Neu5Ac or CMP-Neu5Gc) onto a glycan acceptor. Sialyltransferases comprise alpha-2,3-sialyltransferases, alpha-2,6-sialyltransferases and alpha-2,8-sialyltransferases that catalyse the transfer of a sialic acid onto a glycan acceptor via alpha-glycosidic bonds. Sialyltransferases can be found but are not limited to the GT29, GT42, GT80 and GT97 CAZy families. Galactosyltransferases are glycosyltransferases that transfer a galactosyl group (Gal) from an UDP-galactose (UDP-Gal) donor onto a glycan acceptor. Galactosyltransferases comprise beta-1,3-galactosyltransferases, N-acetylglucosamine beta-1,3-galactosyltransferases, beta-1,4-galactosyltransferases, N-acetylglucosamine beta-1,4-galactosyltransferases, alpha-1,3-galactosyltransferases and alpha-1,4-galactosyltransferases that transfer a Gal residue from UDP-Gal onto a glycan acceptor via alpha- or beta-glycosidic bonds. Galactosyltransferases can be found but are not limited to the GT2, GT6, GT8, GT25 and GT92 CAZy families. Glucosyltransferases are glycosyltransferases that transfer a glucosyl group (Glc) from an UDP-glucose (UDP-Glc) donor onto a glycan acceptor. Glucosyltransferases comprise alpha-glucosyltransferases, beta-1,2-glucosyltransferases, beta-1,3-glucosyltransferases and beta-1,4-glucosyltransferases that transfer a Glc residue from UDP-Glc onto a glycan acceptor via alpha- or beta-glycosidic bonds. Glucosyltransferases can be found but are not limited to the GT1, GT4 and GT25 CAZy families. Mannosyltransferases are glycosyltransferases that transfer a mannose group (Man) from a GDP-mannose (GDP-Man) donor onto a glycan acceptor. Mannosyltransferases comprise alpha-1,2-mannosyltransferases, alpha-1,3-mannosyltransferases and alpha-1,6-mannosyltransferases that transfer a Man residue from GDP-Man onto a glycan acceptor via alpha-glycosidic bonds. Mannosyltransferases can be found but are not limited to the GT22, GT39, GT62 and GT69 CAZy families. N-acetylglucosaminyltransferases are glycosyltransferases that transfer an N-acetylglucosamine group (GlcNAc) from an UDP-N-acetylglucosamine (UDP-GlcNAc) donor onto a glycan acceptor. N-acetylglucosaminyltransferases can be found but are not limited to GT2 and GT4 CAZy families. Galactoside beta-1,3-N-acetylglucosaminyltransferases are part of N-acetylglucosaminyltransferases and transfer GlcNAc from an UDP-GlcNAc donor onto a terminal galactose unit present in a glycan acceptor via a beta-1,3-linkage. Beta-1,6-N-acetylglucosaminyltransferases are N-acetylglucosaminyltransferases that transfer GlcNAc from an UDP-GlcNAc donor onto a glycan acceptor via a beta-1,6-linkage. N-acetylgalactosaminyltransferases are glycosyltransferases that transfer an N-acetylgalactosamine group (GalNAc) from an UDP-N-acetylgalactosamine (UDP-GalNAc) donor onto a glycan acceptor. N-acetylgalactosaminyltransferases can be found but are not limited to GT7, GT12 and GT27 CAZy families. Alpha-1,3-N-acetylgalactosaminyltransferases are part of the N-acetylgalactosaminyltransferases and transfer GalNAc from an UDP-GalNAc donor to a glycan acceptor via an alpha-1,3-linkage. N-acetylmannosaminyltransferases are glycosyltransferases that transfer an N-acetylmannosamine group (ManNAc) from an UDP-N-acetylmannosamine (UDP-ManNAc) donor onto a glycan acceptor. Xylosyltransferases are glycosyltransferases that transfer a xylose residue (Xyl) from an UDP-xylose (UDP-Xyl) donor onto a glycan acceptor. Xylosyltransferases can be found but are not limited to GT14, GT61 and GT77 CAZy families. Glucuronyltransferases are glycosyltransferases that transfer a glucuronate from an UDP-glucuronate donor onto a glycan acceptor via alpha- or beta-glycosidic bonds. Glucuronyltransferases can be found but are not limited to GT4, GT43 and GT93 CAZy families. Galacturonyltransferases are glycosyltransferases that transfer a galacturonate from an UDP-galacturonate donor onto a glycan acceptor. N-glycolylneuraminyltransferases are glycosyltransferases that transfer an N-glycolylneuraminic acid group (Neu5Gc) from a CMP-Neu5Gc donor onto a glycan acceptor. Rhamnosyltransferases are glycosyltransferases that transfer a rhamnose residue from a GDP-rhamnose donor onto a glycan acceptor.

Rhamnosyltransferases can be found but are not limited to the GT1, GT2 and GT102 CAZy families. N-acetylrhamnosyltransferases are glycosyltransferases that transfer an N-acetylrhamnosamine residue from an UDP-N-acetyl-L-rhamnosamine donor onto a glycan acceptor. UDP-4-amino-4,6-dideoxy-N-acetyl-beta-L-altrosamine transaminases are glycosyltransferases that use an UDP-2-acetamido-2,6-dideoxy--L-arabino-4-hexulose in the biosynthesis of pseudaminic acid, which is a sialic acid-like sugar that is used to modify flagellin. UDP-N-acetylglucosamine enolpyruvyl transferases (murA) are glycosyltransferases that transfer an enolpyruvyl group from phosphoenolpyruvate (PEP) to UDP-N-acetylglucosamine (UDPAG) to form UDP-N-acetylglucosamine enolpyruvate. Fucosaminyltransferases are glycosyltransferases that transfer an N-acetylfucosamine residue from a dTDP-N-acetylfucosamine or an UDP-N-acetylfucosamine donor onto a glycan acceptor.

The terms “alpha-2,3-sialyltransferase,” “alpha 2,3 sialyltransferase,” “3-sialyltransferase,” “α-2,3-sialyltransferase,” “a 2,3 sialyltransferase,” “3 sialyltransferase,” “3-ST” or “3ST” as used in the disclosure, are used interchangeably and refer to a glycosyltransferase that catalyzes the transfer of sialic acid from the donor CMP-Neu5Ac, to the acceptor molecule in an alpha-2,3-linkage. The terms “3′ sialyllactose,” “3′-sialyllactose,” “alpha-2,3-sialyllactose,” “alpha 2,3 sialyllactose,” “α-2,3-sialyllactose,” “a 2,3 sialyllactose,” “3SL” or “3′SL” as used in the disclosure, are used interchangeably and refer to the product obtained by the catalysis of the alpha-2,3-fucosyltransferase transferring the sialic acid group from CMP-Neu5Ac to lactose in an alpha-2,3-linkage. The terms “alpha-2,6-sialyltransferase,” “alpha 2,6 sialyltransferase,” “6-sialyltransferase,” “α-2,6-sialyltransferase,” “a 2,6 sialyltransferase,” “6 sialyltransferase,” “6-ST” or “6ST” as used in the disclosure, are used interchangeably and refer to a glycosyltransferase that catalyzes the transfer of sialic acid from the donor CMP-Neu5Ac, to the acceptor molecule in an alpha-2,6-linkage. The terms “6′ sialyllactose,” “6′-sialyllactose,” “alpha-2,6-sialyllactose,” “alpha 2,6 sialyllactose,” “α-2,6-sialyllactose,” “α 2,6 sialyllactose,” “6SL” or “6′SL” as used in the disclosure, are used interchangeably and refer to the product obtained by the catalysis of the alpha-2,6-fucosyltransferase transferring the sialic acid group from CMP-Neu5Ac to lactose in an alpha-2,6-linkage.

The terms “alpha-2,8-sialyltransferase,” “alpha 2,8 sialyltransferase,” “8-sialyltransferase,” “α-2,8-sialyltransferase,” “α 2,8 sialyltransferase,” “8 sialyltransferase,” “8-ST” or “8ST” as used in the disclosure, are used interchangeably and refer to a glycosyltransferase that catalyzes the transfer of sialic acid from the donor CMP-Neu5Ac, to the acceptor in an alpha-2,8-linkage.

The terms “activated monosaccharide,” “nucleotide-activated sugar,” “nucleotide-sugar,” “activated sugar,” “nucleoside” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), CMP-N-glycolylneuraminic acid (CMP-Neu5Gc), CMP-Neu4Ac, CMP-Neu5Ac9N₃, CMP-Neu4,5Ac2, CMP-Neu5,7Ac₂, CMP-Neu5,9Ac2, CMP-Neu5,7(8,9)Ac2, GDP-fucose (GDP-Fuc), GDP-rhamnose and UDP-xylose. Nucleotide-sugars act as glycosyl donors in glycosylation reactions. Glycosylation reactions are reactions that are catalysed by glycosyltransferases.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O—[(R)-1-carboxyethyl]-2-deoxy-D-glucopyranose, 2-Acetamido-3-O—[(R)-carboxyethyl]-2-deoxy-D-glucopyranose, 2-Glycolylamido-3-O—[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, 2-acetamido-2,6-dideoxy-L-arabino-4-hexulose, 2-acetamido-2,6-dideoxy--L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), N-glycolylneuraminic acid, N-acetylgalactosamine (GalNAc), galactosamine (Galn), fucose (Fuc), rhamnose (Rha), glucuronic acid, gluconic acid, fructose (Fru) and polyols.

With the term polyol is meant an alcohol containing multiple hydroxyl groups. For example, glycerol, sorbitol, or mannitol.

The term “disaccharide” as used herein refers to a saccharide polymer containing two simple sugars, i.e., monosaccharides. Such disaccharides contain monosaccharides preferably selected from the list of monosaccharides as used herein above. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc).

The term “sialylated disaccharide” as used herein refers to a disaccharide containing two monosaccharides wherein one of the monosaccharides is a sialic acid as defined herein. Examples of sialylated disaccharides comprise Neu5Ac-a2,3-Gal, Neu5Ac-a2,6-Gal and fucopyranosyl-(1-4)-N-glycolylneuraminic acid (Fuc-(1-4)-Neu5Gc).

“Oligosaccharide” as the term is used herein and as generally understood in the state of the art, refers to a saccharide polymer containing a small number, typically three to twenty, of simple sugars, i.e., monosaccharides. Preferably the oligosaccharide as described herein contains monosaccharides selected from the list as used herein above. The oligosaccharide as used in the disclosure can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc,” “b-Gal-(1->4)-Glc,” “Galbeta1-4-Glc” and “Gal-b(1-4)-Glc” have the same meaning, i.e., a beta-glycosidic bond links carbon-1 of galactose (Gal) with the carbon-4 of glucose (Glc). Each monosaccharide can be in the cyclic form (e.g., pyranose or furanose form). An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically.

Examples of oligosaccharides include but are not limited to Lewis-type antigen oligosaccharides, mammalian (including human) milk oligosaccharides, O-antigen, enterobacterial common antigen (ECA), the glycan chain present in lipopolysaccharides (LPS), the oligosaccharide repeats present in capsular polysaccharides, peptidoglycan (PG), amino-sugars and antigens of the human ABO blood group system.

The term “glycan acceptor” as used herein refers to mono-, di- and oligosaccharides as defined herein.

As used herein, “mammalian milk oligosaccharide” (MMO) refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-triose II, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

A ‘fucosylated oligosaccharide’ as used herein and as generally understood in the state of the art is an oligosaccharide that is carrying a fucose-residue. Examples comprise 2′-fucosyllactose (2′FL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNF I), Lacto-N-fucopentaose II (LNF II), Lacto-N-fucopentaose III (LNF III), lacto-N-fucopentaose V (LNF V), lacto-N-fucopentaose VI (LNF VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose. Mammalian milk oligosaccharides comprise oligosaccharides present in milk found in any phase during lactation including colostrum milk from humans and mammals including but not limited to cows (Bos Taurus), sheep (Ovis aries), goats (Capra aegagrus hircus), bactrian camels (Camelus bactrianus), horses (Equusferus caballus), pigs (Sus scropha), dogs (Canis lupusfamiliaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus). Human milk oligosaccharides (HMOs) are also known as human identical milk oligosaccharides, which are chemically identical to the human milk oligosaccharides found in human breast milk, but which are biotechnologically-produced (e.g., using cell free systems or cells and organisms comprising a bacterium, a fungus, a yeast, a plant, animal, or protozoan cell, preferably genetically engineered cells and organisms). Human identical milk oligosaccharides are marketed under the name HiMO.

As used herein, a ‘sialylated oligosaccharide’ is to be understood as a charged sialic acid containing oligosaccharide, i.e., an oligosaccharide having a sialic acid residue. It has an acidic nature. Some examples are 3-SL (3′-sialyllactose or 3′-SL or Neu5Ac-a2,3-Gal-b1,4-Glc), 3′-sialyllactosamine, 6-SL (6′-sialyllactose or 6′-SL or Neu5Ac-a2,6-Gal-b1,4-Glc), 3,6-disialyllactose (Neu5Ac-a2,3-(Neu5Ac-a2,6)-Gal-b1,4-Glc), 6,6′-disialyllactose (Neu5Ac-a2,6-Gal-b1,4-(Neu5Ac-a2,6)-Glc), 8,3-disialyllactose (Neu5Ac-a2,8-Neu5Ac-a2,3-Gal-b1,4-Glc), 6′-sialyllactosamine, oligosaccharides comprising 6′-sialyllactose, SGG hexasaccharide (Neu5Acα-2,3Galβ-1,3GalNacβ-1,3Galα-1,4Galβ-1,4Gal), sialylated tetrasaccharide (Neu5Acα-2,3Galβ-1,4GlcNacβ-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s), including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GD1a Neu5Acα-2,3Galβ-1,3GalNAcβ-1,4(Neu5Acα-2,3)Galβ-1,4Glc, GT1a Neu5Acα-2,8Neu5Acα-2,3Galβ-1,3GalNAcβ-1,4(Neu5Acα-2,3)Galβ-1,4Glc, GD2 GalNAcβ-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAc β-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAc β-1,4(Neu5Acα-2,8Neu5Acα-2,8Neu5Acα2,3)Galβ-1,4Glc, GP1c Neu5Acα-2,8Neu5Acα-2,3Galβ-1,3GalNAc β-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fucα-1,2Galβ-1,3GalNAcβ-1,4(Neu5Acα-2,3)Gal β-1,4Glc; all of which may be extended to the production of the corresponding gangliosides by reacting the above oligosaccharide moieties with ceramide or synthetizing the above oligosaccharides on a ceramide.

A ‘neutral oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2′, 3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

As used herein the term “Lewis-type antigens” comprise the following oligosaccharides: H1 antigen, which is Fucα1-2Galβ1-3GlcNAc, or in short 2′FLNB; Lewisa, which is the trisaccharide Galβ1-3[Fucα1-4]GlcNAc, or in short 4-FLNB; Lewisb, which is the tetrasaccharide Fucα1-2Galβ1-3[Fucα1-4]GlcNAc, or in short DiF-LNB; sialyl Lewisa, which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H₂ antigen, which is Fucα1-2Galβ1-4GlcNAc, or otherwise stated 2′fucosyl-N-acetyl-lactosamine, in short 2′FLacNAc; Lewisx, which is the trisaccharide Galβ1-4[Fucα1-3]GlcNAc, or otherwise known as 3-Fucosyl-N-acetyl-lactosamine, in short 3-FLacNAc, Lewisy, which is the tetrasaccharide Fucα1-2Galβ1-4[Fucα1-3]GlcNAc and sialyl Lewisx, which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GlcNAc.

As used herein, the term “O-antigen” refers to the repetitive glycan component of the surface lipopolysaccharide (LPS) of Gram-negative bacteria. The term “lipopolysaccharide” or “LPS” refers to glycolipids found in the outer membrane of Gram-negative bacteria, which are composed of a lipid A, a core oligosaccharide and the O-antigen. The term “capsular polysaccharides” refers to long-chain polysaccharides with oligosaccharide repeat structures that are present in bacterial capsules, the latter being a polysaccharide layer that lies outside the cell envelope. The terms “peptidoglycan” or “murein” refers to an essential structural element in the cell wall of most bacteria, being composed of sugars and amino acids, wherein the sugar components consist of alternating residues of beta-1,4 linked GlcNAc and N-acetylmuramic acid. The term “amino-sugar” as used herein refers to a sugar molecule in which a hydroxyl group has been replaced with an amine group. As used herein, an antigen of the human ABO blood group system is an oligosaccharide. Such antigens of the human ABO blood group system are not restricted to human structures. The structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal- that are present on disaccharide core structures comprising Gal-beta1,3-GlcNAc, Gal-beta1,4-GlcNAc, Gal-beta1,3-GalNAc and Gal-beta1,4-Glc.

The terms “LNT II,” “LNT-II,” “LN3,” “lacto-N-triose II,” “lacto-N-triose II,” “lacto-N-triose,” “lacto-N-triose” or “GlcNAcβ1-3Galβ1-4Glc” as used in the disclosure, are used interchangeably.

The terms “LNT,” “lacto-N-tetraose,” “lacto-N-tetraose” or “Galβ1-3GlcNAcβ1-3Galβ1-4Glc” as used in the disclosure, are used interchangeably.

The terms “LNnT,” “lacto-N-neotetraose,” “lacto-N-neotetraose,” “neo-LNT” or “Galβ1-4GlcNAcβ1-3Galβ1-4Glc” as used in the disclosure, are used interchangeably.

The terms “LSTa,” “LS-Tetrasaccharide a,” “Sialyl-lacto-N-tetraose a,” “sialyllacto-N-tetraose a” or “Neu5Ac-a2,3-Gal-b1,3-GlcNAc-b1,3-Gal-b1,4-Glc” as used in the disclosure, are used interchangeably.

The terms “LSTb,” “LS-Tetrasaccharide b,” “Sialyl-lacto-N-tetraose b,” “sialyllacto-N-tetraose b” or “Gal-b1,3-(Neu5Ac-a2,6)-GlcNAc-b1,3-Gal-b1,4-Glc” as used in the disclosure, are used interchangeably.

The terms “LSTc,” “LS-Tetrasaccharide c,” “Sialyl-lacto-N-tetraose c,” “sialyllacto-N-tetraose c,” “sialyllacto-N-neotetraose c” or “Neu5Ac-a2,6-Gal-b1,4-GlcNAc-b1,3-Gal-b1,4-Glc” as used in the disclosure, are used interchangeably.

The terms “LSTd,” “LS-Tetrasaccharide d,” “Sialyl-lacto-N-tetraose d,” “sialyllacto-N-tetraose d,” “sialyllacto-N-neotetraose d” or “Neu5Ac-a2,3-Gal-b1,4-GlcNAc-b1,3-Gal-b1,4-Glc” as used in the disclosure, are used interchangeably.

The terms “DSLNnT” and “Disialyllacto-N-neotetraose” are used interchangeably and refer to Neu5Ac-a2,6-[Neu5Ac-a2,6-Gal-b1,4-GlcNAc-b1,3]-Gal-b1,4-Glc.

The terms “DSLNT” and “Disialyllacto-N-tetraose” are used interchangeably and refer to Neu5Ac-a2,6-[Neu5Ac-a2,3-Gal-b1,3-GlcNAc-b1,3]-Gal-b1,4-Glc. The terms “LNFP-I,” “lacto-N-fucopentaose I,” “LNFP I,” “LNF I OH type I determinant,” “LNF I,” “LNF1,” “LNF 1” and “Blood group H antigen pentaose type 1” are used interchangeably and refer to Fuc-a1,2-Gal-b1,3-GlcNAc-b1,3-Gal-b1,4-Glc.

The terms “LNB” and “Lacto-N-biose” are used interchangeably and refer to the disaccharide Gal-b1,3-GlcNAc.

The terms “LacNAc” and “N-acetyllactosamine” are used interchangeably and refer to the disaccharide Gal-b 1,4-GlcNAc.

The term “membrane transporter proteins” as used herein refers to proteins that are part of or interact with the cell membrane and control the flow of molecules and information across the cell. The membrane proteins are thus involved in transport, be it import into or export out of the cell.

The term “Siderophore” as used herein is referring to the secondary metabolite of various microorganisms, which are mainly ferric ion specific chelators. These molecules have been classified as catecholate, hydroxamate, carboxylate and mixed types. Siderophores are in general synthesized by a nonribosomal peptide synthetase (NRPS) dependent pathway or an NRPS independent pathway (NIS). The most important precursor in NRPS-dependent siderophore biosynthetic pathway is chorismate. 2,3-DHBA could be formed from chorismate by a three-step reaction catalysed by isochorismate synthase, isochorismatase, and 2,3-dihydroxybenzoate-2,3-dehydrogenase. Siderophores can also be formed from salicylate, which is formed from isochorismate by isochorismate pyruvate lyase. When ornithine is used as precursor for siderophores, biosynthesis depends on the hydroxylation of ornithine catalysed by L-ornithine N5-monooxygenase. In the NIS pathway, an important step in siderophore biosynthesis is N(6)-hydroxylysine synthase.

A transporter is needed to export the siderophore outside the cell. Four superfamilies of membrane proteins are identified so far in this process: the major facilitator superfamily (MFS); the Multidrug/Oligosaccharidyl-lipid/Polysaccharide Flippase Superfamily (MOP); the resistance, nodulation and cell division superfamily (RND); and the ABC superfamily. In general, the genes involved in siderophore export are clustered together with the siderophore biosynthesis genes. The term “siderophore exporter” as used herein refers to such transporters needed to export the siderophore outside of the cell.

The ATP-binding cassette (ABC) superfamily contains both uptake and efflux transport systems, and the members of these two porter groups generally cluster loosely together. ATP hydrolysis without protein phosphorylation energizes transport. There are dozens of families within the ABC superfamily, and family generally correlates with substrate specificity. Members are classified according to class 3.A.1 as defined by the Transporter Classification Database operated by the Saier Lab Bioinformatics Group available via www.tcdb.org and providing a functional and phylogenetic classification of membrane transporter proteins.

The major facilitator superfamily (MFS) is a superfamily of membrane transporter proteins catalysing uniport, solute:cation (H+, but seldom Na+) symport and/or solute:H+ or solute:solute antiport. Most are of 400-600 amino acyl residues in length and possess either 12, 14, or occasionally, 24 transmembrane α-helical spanners (TMSs) as defined by the Transporter Classification Database operated by the Saier Lab Bioinformatics Group (www.tcdb.org).

“SET” or “Sugar Efflux Transporter” as used herein refers to membrane proteins of the SET family, which are proteins with InterPRO domain IPR004750 and/or are proteins that belong to the eggNOGv4.5 family ENOG410XTE9. Identification of the InterPro domain can be done by using the online tool on www.ebi.ac.uk/interpro/ or a standalone version of InterProScan (www.ebi.ac.uk/interpro/download.html) using the default values. Identification of the orthology family in eggNOGv4.5 can be done using the online version or a standalone version of eggNOG-mapperv1 (eggnogdb.embl.de/#/app/home).

It should be understood for those skilled in the art that for the databases used herein, comprising eggnogdb 4.5.1 (released September 2016) and InterPro 75.0 (released 4 Jul. 2019), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

The terms “cell genetically modified for the production of a sialylated di- and/or oligosaccharide” within the context of the present disclosure refers to a cell that is genetically manipulated to comprise at least one sialyltransferase combined with any one or more of i) a gene encoding a glycosyltransferase necessary for the synthesis of the sialylated di- and/or oligosaccharide, ii) a biosynthetic pathway to produce a nucleotide donor suitable to be transferred by the glycosyltransferase to a carbohydrate precursor, and/or iii) a biosynthetic pathway to produce a precursor or a mechanism of internalization of a precursor from the culture medium into the cell where it is glycosylated to produce the sialylated di- and/or oligosaccharide.

The term “pathway for production of a sialylated di- and/or oligosaccharide” as used herein is a biochemical pathway consisting of the enzymes and their respective genes involved in the synthesis of a sialylated di- and/or oligosaccharide as defined herein. The pathway for production of a sialylated di- and/or oligosaccharide comprises at least one sialyltransferase. Furthermore, the pathway for production of a sialylated di- and/or oligosaccharide can comprise but is not limited to pathways involved in the synthesis of a nucleotide-activated sugar and the transfer of the nucleotide-activated sugar to an acceptor to create a sialylated di- and/or oligosaccharide of the disclosure. Examples of such pathway comprise but are not limited to a fucosylation, sialylation, galactosylation, N-acetylglucosaminylation, N-acetylgalactosylation, mannosylation, N-acetylmannosaminylation pathway.

A ‘fucosylation pathway’ as used herein is a biochemical pathway comprising at least one of the enzymes and their respective genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase combined with a fucosyltransferase leading to a 1,2; a 1,3; a 1,4 and/or a 1,6 fucosylated oligosaccharides.

A ‘sialylation pathway’ is a biochemical pathway comprising at least one of the enzymes and their respective genes chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-acylneuraminate-9-phosphate synthetase, phosphatase, N-acetylneuraminate synthase, N-acylneuraminate cytidylyltransferase and sialic acid transporter combined with a sialyltransferase leading to a 2,3; a 2,6 and/or α2,8 sialylated oligosaccharides.

A ‘galactosylation pathway’ as used herein is a biochemical pathway comprising at least one of the enzymes and their respective genes chosen from the list comprising galactose-1-epimerase, galactokinase, glucokinase, galactose-1-phosphate uridylyltransferase, UDP-glucose 4-epimerase, glucose-1-phosphate uridylyltransferase, glucophosphomutase combined with a galactosyltransferase leading to a galactosylated compound comprising a mono-, di-, or oligosaccharide having an alpha or beta bound galactose on any one or more of the 2, 3, 4 and 6 hydroxyl group of the mono-, di-, or oligosaccharide.

An ‘N-acetylglucosaminylation pathway’ as used herein is a biochemical pathway comprising at least one of the enzymes and their respective genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase combined with a glycosyltransferase leading to a GlcNAc-modified compound comprising a mono-, di-, or oligosaccharide having an alpha or beta bound N-acetylglucosamine (GlcNAc) on any one or more of the 3, 4 and 6 hydroxyl group of the mono-, di- or oligosaccharide.

An ‘N-acetylgalactosylation pathway’ as used herein is a biochemical pathway comprising at least one of the enzymes and their respective genes chosen from the list comprising L-glutamine-D-fructose-6-phosphate aminotransferase, phosphoglucosamine mutase, N-acetylglucosamine 1-phosphate uridylyltransferase, UDP-N-acetylglucosamine 4-epimerase, UDP-galactose 4-epimerase, N-acetylgalactosamine kinase and/or UDP-GalNAc pyrophosphorylase combined with a glycosyltransferase leading to a GalNAc-modified compound comprising a mono-, di- or oligosaccharide having an alpha or beta bound N-acetylgalactosamine on the mono-, di- or oligosaccharide.

A ‘mannosylation pathway’ as used herein is a biochemical pathway comprising at least one of the enzymes and their respective genes chosen from the list comprising mannose-6-phosphate isomerase, phosphomannomutase and/or mannose-1-phosphate guanyltransferase combined with a glycosyltransferase leading to a mannosylated compound comprising a mono-, di- or oligosaccharide having an alpha or beta bound mannose on the mono-, di- or oligosaccharide.

An ‘N-acetylmannosaminylation pathway’ as used herein is a biochemical pathway comprising at least one of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase combined with a glycosyltransferase leading to a ManNAc-modified compound comprising a mono-, di- or oligosaccharide having an alpha or beta bound N-acetylmannosamine on the mono-, di- or oligosaccharide.

The terms “mannose-6-phosphate isomerase,” “phosphomannose isomerase,” “mannose phosphate isomerase,” “phosphohexoisomerase,” “phosphomannoisomerase,” “phosphomannose-isomerase,” “phosphohexomutase,” “D-mannose-6-phosphate ketol-isomerase”and “manA” are used interchangeably and refer to an enzyme that catalyses the reversible conversion of D-fructose 6-phosphate to D-mannose 6-phosphate.

The terms “phosphomannomutase,” “mannose phosphomutase,” “phosphomannose mutase,” “D-mannose 1,6-phosphomutase” and “manB” are used interchangeably and refer to an enzyme that catalyses the reversible conversion of D-mannose 6-phosphate to D-mannose 1-phosphate.

The terms “mannose-1-phosphate guanylyltransferase,” “GTP-mannose-1-phosphate guanylyltransferase,” “PIM-GMP (phosphomannose isomerase-guanosine 5′-diphospho-D-mannose pyrophosphorylase),” “GDP-mannose pyrophosphorylase,” “guanosine 5′-diphospho-D-mannose pyrophosphorylase,” “guanosine diphosphomannose pyrophosphorylase,” “guanosine triphosphate-mannose 1-phosphate guanylyltransferase,” “mannose 1-phosphate guanylyltransferase (guanosine triphosphate)” and “manC” are used interchangeably and refer to an enzyme that converts D-mannose-1-phosphate using GTP into GDP-mannose and diphosphate.

The terms “GDP-mannose 4,6-dehydratase,” “guanosine 5′-diphosphate-D-mannose oxidoreductase,” “guanosine diphosphomannose oxidoreductase,” “guanosine diphosphomannose 4,6-dehydratase,” “GDP-D-mannose dehydratase,” “GDP-D-mannose 4,6-dehydratase,” “GDP-mannose 4,6-hydro-lyase,” “GDP-mannose 4,6-hydro-lyase (GDP-4-dehydro-6-deoxy-D-mannose-forming)” and “gmd” are used interchangeably and refer to an enzyme that forms the first step in the biosynthesis of GDP-rhamnose and GDP-fucose.

The terms “GDP-L-fucose synthase,” “GDP-4-keto-6-deoxy-D-mannose-3,5-epimerase-4-reductase,” “GDP-L-fucose:NADP+4-oxidoreductase (3,5-epimerizing)” and “fcl” are used interchangeably and refer to an enzyme that forms the second step in the biosynthesis of GDP-fucose.

The terms “L-fucokinase/GDP-fucose pyrophosphorylase,” “L-fucokinase/L-fucose-1-P guanylyltransferase,” “GDP-fucose pyrophosphorylase,” “GDP-L-fucose pyrophosphorylase,” and “fkp” are used interchangeably and refer to an enzyme that catalyses the conversion of L-fucose-1-phosphate into GDP-fucose using GTP.

The terms “L-glutamine-D-fructose-6-phosphate aminotransferase,” “glutamine---fructose-6-phosphate transaminase (isomerizing),” “hexosephosphate aminotransferase,” “glucosamine-6-phosphate isomerase (glutamine-forming),” “glutamine-fructose-6-phosphate transaminase (isomerizing),” “D-fructose-6-phosphate amidotransferase,” “fructose-6-phosphate aminotransferase,” “glucosaminephosphate isomerase,” “glucosamine 6-phosphate synthase,” “GlcN6P synthase,” “GFA,” “glms,” “glmS” and “glmS*54” are used interchangeably and refer to an enzyme that catalyses the conversion of D-fructose-6-phosphate into D-glucosamine-6-phosphate using L-glutamine.

The terms “glucosamine-6-P deaminase,” “glucosamine-6-phosphate deaminase,” “GlcN6P deaminase,” “glucosamine-6-phosphate isomerase,” “glmD” and “nagB” are used interchangeably and refer to an enzyme that catalyses the reversible isomerization-deamination of glucosamine-6-phosphate (GlcN6P) to form fructose-6-phosphate and an ammonium ion.

The terms “phosphoglucosamine mutase” and “glmM” are used interchangeably and refer to an enzyme that catalyses the conversion of glucosamine-6-phosphate to glucosamine-1-phosphate. Phosphoglucosamine mutase can also catalyse the formation of glucose-6-P from glucose-1-P, although at a 1400-fold lower rate.

The terms “N-acetylglucosamine-6-P deacetylase,” “N-acetylglucosamine-6-phosphate deacetylase” and “nagA” are used interchangeably and refer to an enzyme that catalyses the hydrolysis of the N-acetyl group of N-acetylglucosamine-6-phosphate (GlcNAc-6-P) to yield glucosamine-6-phosphate (GlcN6P) and acetate.

An N-acylglucosamine 2-epimerase is an enzyme that catalyses the reaction N-acyl-D-glucosamine=N-acyl-D-mannosamine. Alternative names for this enzyme comprise N-acetylglucosamine 2-epimerase, N-acetyl-D-glucosamine 2-epimerase, GlcNAc 2-epimerase, N-acyl-D-glucosamine 2-epimerase and N-acetylglucosamine epimerase.

An UDP-N-acetylglucosamine 2-epimerase is an enzyme that catalyses the reaction N-acetyl-D-glucosamine=N-acetylmannosamine. Alternative names for this enzyme comprise UDP-N-acylglucosamine 2-epimerase, UDP-GlcNAc-2-epimerase and UDP-N-acetyl-D-glucosamine 2-epimerase.

An N-acetylmannosamine-6-phosphate 2-epimerase is an enzyme that catalyses the reaction N-acetyl-D-glucosamine 6-phosphate=N-acetyl-D-mannosamine 6-phosphate.

A bifunctional UDP-GlcNAc 2-epimerase/kinase is a bifunctional enzyme that catalyses the reaction UDP-N-acetyl-D-glucosamine=N-acetyl-D-mannosamine and the reaction N-acetyl-D-mannosamine+ATP=ADP+N-acetyl-D-mannosamine 6-phosphate.

A glucosamine 6-phosphate N-acetyltransferase is an enzyme that catalyses the transfer of an acetyl group from acetyl-CoA to D-glucosamine-6-phosphate thereby generating a free CoA and N-acetyl-D-glucosamine 6-phosphate. Alternative names comprise aminodeoxyglucosephosphate acetyltransferase, D-glucosamine-6-P N-acetyltransferase, glucosamine 6-phosphate acetylase, glucosamine 6-phosphate N-acetyltransferase, glucosaminephosphate N-acetyltransferase, glucosamine-6-phosphate acetylase, N-acetylglucosamine-6-phosphate synthase, phosphoglucosamine acetylase, phosphoglucosamine N-acetylase phosphoglucosamine N-acetylase, phosphoglucosamine transacetylase, GNA and GNA1.

The term “N-acetylglucosamine-6-phosphate phosphatase” refers to an enzyme that dephosphorylates N-acetylglucosamine-6-phosphate (GlcNAc-6-P) hereby synthesizing N-acetylglucosamine (GlcNAc).

The term “N-acetylmannosamine-6-phosphate phosphatase” refers to an enzyme that dephosphorylates N-acetylmannosamine-6-phosphate (ManNAc-6P) to N-acetylmannosamine (ManNAc).

The terms “N-acetylmannosamine-6-phosphate 2-epimerase,” “ManNAc-6-P isomerase,” “ManNAc-6-P 2-epimerase,” N-acetylglucosamine-6P 2-epimerase and “nanE” are used interchangeably and refer to an enzyme that converts ManNAc-6-P to N-acetylglucosamine-6-phosphate (GlcNAc-6-P).

The terms “phosphoacetylglucosamine mutase,” “acetylglucosamine phosphomutase,” “acetylaminodeoxyglucose phosphomutase,” “phospho-N-acetylglucosamine mutase” and “N-acetyl-D-glucosamine 1,6-phosphomutase” are used interchangeably and refer to an enzyme that catalyses the conversion of N-acetyl-glucosamine 1-phosphate into N-acetylglucosamine 6-phosphate.

The terms “N-acetylglucosamine 1-phosphate uridylyltransferase,” “N-acetylglucosamine-1-phosphate uridyltransferase,” “UDP-N-acetylglucosamine diphosphorylase,” “UDP-N-acetylglucosamine pyrophosphorylase,” “uridine diphosphoacetylglucosamine pyrophosphorylase,” “UTP:2-acetamido-2-deoxy-alpha-D-glucose-1-phosphate uridylyltransferase,” “UDP-GlcNAc pyrophosphorylase,” “GlmU uridylyltransferase,” “Acetylglucosamine 1-phosphate uridylyltransferase,” “UDP-acetylglucosamine pyrophosphorylase,” “uridine diphosphate-N-acetylglucosamine pyrophosphorylase,” “uridine diphosphoacetylglucosamine phosphorylase,” and “acetylglucosamine 1-phosphate uridylyltransferase” are used interchangeably and refer to an enzyme that catalyses the conversion of N-acetylglucosamine 1-phosphate (GlcNAc-1-P) into UDP-N-acetylglucosamine (UDP-GlcNAc) by the transfer of uridine 5-monophosphate (from uridine 5-triphosphate (UTP)).

The term glucosamine-1-phosphate acetyltransferase refers to an enzyme that catalyses the transfer of the acetyl group from acetyl coenzyme A to glucosamine-1-phosphate (GlcN-1-P) to produce N-acetylglucosamine-1-phosphate (GlcNAc-1-P).

The term “glmU” refers to a bifunctional enzyme that has both N-acetylglucosamine-1-phosphate uridyltransferase and glucosamine-1-phosphate acetyltransferase activity and that catalyses two sequential reactions in the de novo biosynthetic pathway for UDP-GlcNAc. The C-terminal domain catalyses the transfer of acetyl group from acetyl coenzyme A to GlcN-1-P to produce GlcNAc-1-P, which is converted into UDP-GlcNAc by the transfer of uridine 5-monophosphate, a reaction catalysed by the N-terminal domain.

The terms “NeunAc synthase,” “N-acetylneuraminic acid synthase,” “N-acetylneuraminate synthase,” “sialic acid synthase,” “NeuAc synthase,” “NeuB,” “NeuB1,” “NeuNAc synthase,” “NANA condensing enzyme,” “N-acetylneuraminate lyase synthase,” “N-acetylneuraminic acid condensing enzyme” as used herein are used interchangeably and refer to an enzyme capable to synthesize sialic acid from N-acetylmannosamine (ManNAc) in a reaction using phosphoenolpyruvate (PEP).

The terms “N-acetylneuraminate lyase,” “Neu5Ac lyase,” “N-acetylneuraminate pyruvate-lyase,” “N-acetylneuraminic acid aldolase,” “NALase,” “sialate lyase,” “sialic acid aldolase,” “sialic acid lyase” and “nanA” are used interchangeably and refer to an enzyme that degrades N-acetylneuraminate into N-acetylmannosamine (ManNAc) and pyruvate.

The terms “N-acylneuraminate-9-phosphate synthase,” “N-acylneuraminate-9-phosphate synthetase,” “NANA synthase,” “NANAS,” “NANS,” “NmeNANAS,” “N-acetylneuraminate pyruvate-lyase (pyruvate-phosphorylating)” as used herein are used interchangeably and refer to an enzyme capable to synthesize N-acylneuraminate-9-phosphate from N-acetylmannosamine-6-phosphate (ManNAc-6-phosphate) in a reaction using phosphoenolpyruvate (PEP).

The term “N-acylneuraminate-9-phosphatase” refers to an enzyme capable to dephosphorylate N-acylneuraminate-9-phosphate to synthesise N-acylneuraminate.

The terms “CMP-sialic acid synthase,” “N-acylneuraminate cytidylyltransferase,” “CMP-sialate synthase,” “CMP-NeuAc synthase,” “NeuA” and “CMP-N-acetylneuraminic acid synthase” as used herein are used interchangeably and refer to an enzyme capable to synthesize CMP-N-acetylneuraminate from N-acetylneuraminate using CTP in the reaction.

The terms “galactose-1-epimerase,” “aldose 1-epimerase,” “mutarotase,” “aldose mutarotase,” “galactose mutarotase,” “galactose 1-epimerase” and “D-galactose 1-epimerase” are used interchangeably and refer to an enzyme that catalyses the conversion of beta-D-galactose into alpha-D-galactose.

The terms “galactokinase,” “galactokinase (phosphorylating)” and “ATP:D-galactose-1-phosphotransferase” are used interchangeably and refer to an enzyme that catalyses the conversion of alpha-D-galactose into alpha-D-galactose 1-phosphate using ATP.

The terms glucokinase, and “glucokinase (phosphorylating)” are used interchangeably and refer to an enzyme that catalyses the conversion of D-glucose into D-glucose 6-phosphate using ATP.

The terms “galactose-1-phosphate uridylyltransferase,” “Gal-1-P uridylyltransferase,” “UDP-glucose---hexose-1-phosphate uridylyltransferase,” “uridyl transferase,” “hexose-1-phosphate uridylyltransferase,” “uridyltransferase”; “hexose 1-phosphate uridyltransferase,” “UDP-glucose:alpha-D-galactose-1-phosphate uridylyltransferase,” “galB” and “galT” are used interchangeably and refer to an enzyme that catalyses the reaction D-galactose 1-phosphate+UDP-D-glucose=D-glucose 1-phosphate+UDP-D-galactose.

The terms “UDP-glucose 4-epimerase,” “UDP-galactose 4-epimerase,” “uridine diphosphoglucose epimerase,” “galactowaldenase,” “UDPG-4-epimerase,” “uridine diphosphate galactose 4-epimerase,” “uridine diphospho-galactose-4-epimerase,” “UDP-glucose epimerase,” “4-epimerase,” “uridine diphosphoglucose 4-epimerase,” “uridine diphosphate glucose 4-epimerase” and “UDP-D-galactose 4-epimerase” are used interchangeably and refer to an enzyme that catalyses the conversion of UDP-D-glucose into UDP-galactose.

The terms “glucose-1-phosphate uridylyltransferase,” “UTP---glucose-1-phosphate uridylyltransferase,” “UDP glucose pyrophosphorylase,” “UDPG phosphorylase,” “UDPG pyrophosphorylase,” “uridine 5′-diphosphoglucose pyrophosphorylase,” “uridine diphosphoglucose pyrophosphorylase,” “uridine diphosphate-D-glucose pyrophosphorylase,” “uridine-diphosphate glucose pyrophosphorylase” and “galU” are used interchangeably and refer to an enzyme that catalyses the conversion of D-glucose-1-phosphate into UDP-glucose using UTP.

The terms “phosphoglucomutase (alpha-D-glucose-1,6-bisphosphate-dependent),” “glucose phosphomutase (ambiguous)” and “phosphoglucose mutase (ambiguous)” are used interchangeably and refer to an enzyme that catalyses the conversion of D-glucose 1-phosphate into D-glucose 6-phosphate.

The terms “UDP-N-acetylglucosamine 4-epimerase,” “UDP acetylglucosamine epimerase,” “uridine diphosphoacetylglucosamine epimerase,” “uridine diphosphate N-acetylglucosamine-4-epimerase,” “uridine 5′-diphospho-N-acetylglucosamine-4-epimerase” and “UDP-N-acetyl-D-glucosamine 4-epimerase” are used interchangeably and refer to an enzyme that catalyses the epimerization of UDP-N-acetylglucosamine (UDP-GlcNAc) to UDP-N-acetylgalactosamine (UDP-GalNAc).

The terms “N-acetylgalactosamine kinase,” “GALK2,” “GK2,” “GalNAc kinase,” “N-acetylgalactosamine (GalNAc)-1-phosphate kinase” and “ATP:N-acetyl-D-galactosamine 1-phosphotransferase” are used interchangeably and refer to an enzyme that catalyses the synthesis of N-acetylgalactosamine 1-phosphate (GalNAc-1-P) from N-acetylgalactosamine (GalNAc) using ATP.

The terms “UDP-N-acetylgalactosamine pyrophosphorylase” and “UDP-GalNAc pyrophosphorylase” are used interchangeably and refer to an enzyme that catalyses the conversion of N-acetylgalactosamine 1-phosphate (GalNAc-1-P) into UDP-N-acetylgalactosamine (UDP-GalNAc) using UTP.

The terms “N-acetylneuraminate kinase,” “ManNAc kinase,” “N-acetyl-D-mannosamine kinase” and “nanK” are used interchangeably and refer to an enzyme that phosphorylates ManNAc to synthesize N-acetylmannosamine-phosphate (ManNAc-6-P).

The terms “acetyl-coenzyme A synthetase,” “acs,” “acetyl-CoA synthetase,” “AcCoA synthetase,” “acetate--CoA ligase,” “acyl-activating enzyme” and “yfaC” are used interchangeably and refer to an enzyme that catalyses the conversion of acetate into acetyl-coezyme A (AcCoA) in an ATP-dependent reaction.

The terms “pyruvate dehydrogenase,” “pyruvate oxidase,” “POX,” “poxB” and “pyruvate:ubiquinone-8 oxidoreductase” are used interchangeably and refer to an enzyme that catalyses the oxidative decarboxylation of pyruvate to produce acetate and CO₂.

The terms “lactate dehydrogenase,” “D-lactate dehydrogenase,” “ldhA,” “hslI,” “htpH,” “D-LDH,” “fermentative lactate dehydrogenase” and “D-specific 2-hydroxyacid dehydrogenase” are used interchangeably and refer to an enzyme that catalyses the conversion of lactate into pyruvate hereby generating NADH.

The term “enabled efflux” means to introduce the activity of transport of a solute over the cytoplasm membrane and/or the cell wall. The transport may be enabled by introducing and/or increasing the expression of a transporter protein as described in the disclosure. The term “enhanced efflux” means to improve the activity of transport of a solute over the cytoplasm membrane and/or the cell wall. Transport of a solute over the cytoplasm membrane and/or cell wall may be enhanced by introducing and/or increasing the expression of a membrane transporter protein as described in the disclosure. “Expression” of a membrane transporter protein is defined as “overexpression” of the gene encoding the membrane transporter protein in the case the gene is an endogenous gene or “expression” in the case the gene encoding the membrane transporter protein is a heterologous gene that is not present in the wild type strain or cell.

As used herein, the term “cell productivity index (CPI)” refers to the mass of the product produced by the cells divided by the mass of the cells produced in the culture.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as but not limited to thin layer chromatography, gas chromatography, NMR, HPLC, capillary electrophoresis or mass spectroscopy.

The term “cultivation” refers to the culture medium wherein the cell is cultivated or fermented, the cell itself, and the sialylated di- and/or oligosaccharide that is produced by the cell in whole broth, i.e., inside (intracellularly) as well as outside (extracellularly) of the cell.

The term “precursor” as used herein refers to substances that are taken up or synthetized by the cell for the specific production of sialylated di- and/or oligosaccharide according to the disclosure. In this sense a precursor can be an acceptor as defined herein, but can also be another substance, metabolite, which is first modified within the cell as part of the biochemical synthesis route of the sialylated di- and/or oligosaccharide. Examples of such precursors comprise the acceptors as defined herein, and/or glucose, galactose, fructose, glycerol, sialic acid, fucose, mannose, maltose, sucrose, lactose, dihydroxyacetone, glucosamine, N-acetyl-glucosamine, mannosamine, N-acetyl-mannosamine, galactosamine, N-acetylgalactosamine, phosphorylated sugars like e.g., but not limited to glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetyl-glucosamine-6-phosphate, N-acetylmannosamine-6-phosphate, N-acetylglucosamine-1-phosphate, N-acetyl-neuraminic acid-9-phosphate and/or nucleotide-activated sugars as defined herein like e.g., UDP-glucose, UDP-galactose, UDP-N-acetylglucosamine, CMP-sialic acid, GDP-mannose, GDP-4-dehydro-6-deoxy-α-D-mannose, GDP-fucose.

Optionally, the cell is transformed to comprise and to express at least one nucleic acid sequence encoding a protein selected from the group consisting of lactose transporter, fucose transporter, transporter for a nucleotide-activated sugar wherein the transporter internalizes a to the medium added precursor for the synthesis of the sialylated di- and/or oligosaccharide of disclosure.

The term “acceptor” as used herein refers to a mono-, di- or oligosaccharide, which can be modified by a glycosyltransferase. Examples of such acceptors comprise glucose, galactose, fructose, glycerol, sialic acid, fucose, mannose, maltose, sucrose, lactose, lacto-N-triose, lacto-N-tetraose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and oligosaccharide containing 1 or more N-acetyllactosamine units and/or 1 or more lacto-N-biose units or an intermediate into oligosaccharide, fucosylated and sialylated versions thereof.

### DETAILED DESCRIPTION

According to a first embodiment, the disclosure provides a metabolically engineered cell for the production of a sialylated di- and/or oligosaccharide. Herein, a metabolically engineered cell comprising a pathway for the production of a sialylated di- and/or oligosaccharide is provided, which is modified for expression and/or overexpression of multiple coding DNA sequences encoding one or more proteins that catalyse the same chemical reaction.

According to a second embodiment, the disclosure provides a method for the production of a sialylated di- and/or oligosaccharide by a metabolically engineered cell. The method comprises the steps of:
1) providing a cell as described herein, and
2) cultivating the cell under conditions permissive to produce the sialylated di- and/or oligosaccharide.

Preferably, the sialylated di- and/or oligosaccharide is separated from the cultivation as explained herein.

In the scope of the disclosure, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of 30+/−20 degrees centigrade, a pH-range of 7+/−3.

According to one aspect of the method and/or cell of the disclosure, the one or more proteins that are encoded in the cell by expression and/or overexpression of multiple coding DNA sequences and that catalyze the same chemical reaction are isoproteins. Isoproteins are alternative forms of the same protein activity that can differ in any one or more of amino acid composition, sequence, three-dimensional structure, multimeric quaternary structure, protein stability, regulatory properties and kinetic parameters comprising K_{M}, k_{cat}, catalytic efficiency, enzymatic rate and velocity. Isoproteins may have different catalytic efficiencies to catalyse the same chemical reaction.

According to an alternative aspect of the method and/or cell of the disclosure, the one or more proteins that are encoded in the cell by expression and/or overexpression of multiple coding DNA sequences and that catalyze the same chemical reaction are proteins that are involved in the synthesis of a nucleotide-activated sugar to be used in the production of a sialylated di- and/or oligosaccharide.

According to an alternative aspect of the method and/or cell of the disclosure, the one or more proteins that are encoded in the cell by expression and/or overexpression of multiple coding DNA sequences and that catalyze the same chemical reaction are membrane transporter proteins. According to a preferred embodiment of the method and/or cell of the disclosure, the one or more membrane transporter proteins is/are chosen from the list comprising a siderophore exporter, an ABC transporter, an MFS transporter and Sugar Efflux Transporter as defined herein.

The disclosure provides different types of cells for the production of a sialylated di- and/or oligosaccharide with a metabolically engineered cell.

In a preferred embodiment of the method and/or cell of the disclosure, the cell expresses one protein that is expressed by multiple coding DNA sequences. In an alternative and/or additional preferred embodiment of the method and/or cell of the disclosure, the cell expresses two isoproteins that are expressed by multiple coding DNA sequences. In an alternative and/or additional preferred embodiment of the method and/or cell of the disclosure, the cell expresses three or more isoproteins that are expressed by multiple coding DNA sequences.

In a preferred embodiment of the method and/or cell of disclosure, the cell is metabolically engineered to comprise a pathway for the production of a sialylated di- and/or oligosaccharide as defined herein. In an alternative preferred embodiment of the method and/or cell of disclosure, the cell is metabolically engineered to comprise a pathway for the production of a sialylated di- and/or oligosaccharide and to express and/or overexpress any one or more isoproteins that catalyze the same chemical reaction.

According to a preferred aspect of the method and/or cell of the disclosure, the protein and/or two or more isoproteins that is/are encoded by expression and/or overexpression of multiple coding DNA sequences in the cell is/are involved in the pathway for the production of a sialylated di- and/or oligosaccharide.

In a further preferred aspect of the method and/or cell of the disclosure, the pathway for the production of a sialylated di- and/or oligosaccharide comprises a sialylation pathway as defined herein.

According to a preferred aspect of the method and/or cell of the disclosure, the cell comprises a pathway for production of a sialylated di- and/or oligosaccharide wherein the pathway comprises at least one protein chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-aceylneuraminate-9-phosphate synthetase, phosphatase, N-acetylneuraminate synthase, N-acylneuraminate cytidylyltransferase, sialyltransferase and sialic acid transporter.

According to a more preferred aspect of the method and/or cell of the disclosure, the cell expresses at least one protein chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-aceylneuraminate-9-phosphate synthetase, phosphatase, N-acetylneuraminate synthase, N-acylneuraminate cytidylyltransferase, sialyltransferase and sialic acid transporter wherein the at least one protein is encoded by the multiple coding DNA sequences.

According to another more preferred aspect of the method and/or cell of the disclosure, any one of the N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-aceylneuraminate-9-phosphate synthetase, phosphatase, N-acetylneuraminate synthase, N-acylneuraminate cytidylyltransferase, sialyltransferase and sialic acid transporter is an endogenous protein of the cell with a modified expression or activity, preferably any one of the endogenous N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-aceylneuraminate-9-phosphate synthetase, phosphatase, N-acetylneuraminate synthase, N-acylneuraminate cytidylyltransferase, sialyltransferase and sialic acid transporter is overexpressed; alternatively any one of the N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-aceylneuraminate-9-phosphate synthetase, phosphatase, N-acetylneuraminate synthase, N-acylneuraminate cytidylyltransferase, sialyltransferase and sialic acid transporter is an heterologous protein that is heterogeneously introduced and expressed in the cell, preferably overexpressed. Any one of the endogenous N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-aceylneuraminate-9-phosphate synthetase, phosphatase, N-acetylneuraminate synthase, N-acylneuraminate cytidylyltransferase, sialyltransferase and sialic acid transporter can have a modified expression in the cell, which also expresses any one of a heterologous N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-aceylneuraminate-9-phosphate synthetase, phosphatase, N-acetylneuraminate synthase, N-acylneuraminate cytidylyltransferase, sialyltransferase and sialic acid transporter.

In a preferred embodiment of the method and/or cell, the cell comprises a pathway for production of a sialylated di- and/or oligosaccharide wherein the cell expresses a sialic acid transporter that is e.g., a porter or a P-P-bond-hydrolysis-driven transporter as defined by the Transporter Classification Database that is operated and curated by the Saier Lab Bioinformatics Group available via www.tcdb.org., like e.g., nanT from E. coli with SEQ ID NO:08. Sialic acid can be added to the cell or can be provided by an enzyme expressed in the cell or by the mechanism of the cell as described herein.

In another and/or additional preferred embodiment of the method and/or cell, the cell comprises a pathway for production of a sialylated di- and/or oligosaccharide wherein the cell expresses a sialyltransferase chosen from the list comprising alpha-2,3-sialyltransferase, alpha-2,6-sialyltransferase, and alpha-2,8-sialyltransferase, which transfers sialic acid from CMP-sialic acid to one or more glycan acceptor(s) to produce a sialylated di- and/or oligosaccharide. In another and/or additional preferred embodiment of the method and/or cell, the cell expresses more than one sialyltransferase that synthesize any one or more sialylated di- and/or oligosaccharides as defined herein. CMP-sialic acid can be added to the cell and/or can be provided by an enzyme expressed in the cell or by the mechanism of the cell. Such cell producing CMP-sialic acid is described herein. Preferably, the cell is modified to produce CMP-sialic acid as described herein. More preferably, the cell is modified for enhanced production of CMP-sialic acid as described herein.

In an alternative and/or additional preferred embodiment, the cell comprises a pathway for production of a sialylated di- and/or oligosaccharide wherein the cell expresses an N-acylneuraminate cytidylyltransferase like is known e.g., from N. meningitidis, Homo sapiens, R. norvegicus, Streptomyces sp., C. jejuni, which converts sialic acid into CMP-sialic acid, and a sialyltransferase including an alpha-2,3-sialyltransferase, an alpha-2,6-sialyltransferase and/or an alpha-2,8-sialyltransferase. Sialic acid can be added to the cell and/or can be provided by an enzyme expressed in the cell or by the mechanism of the cell. Such cell producing sialic acid can express an N-acetylneuraminate synthase like is known e.g., from several species including N. meningitidis and C. jejuni. Preferably, the cell is modified to produce sialic acid. More preferably, the cell is modified for enhanced production of sialic acid. The modification can be any one or more chosen from the group comprising knockout of an N-acetylneuraminate lyase, over-expression of an N-acetylneuraminate synthase and over-expression of an N-acetylneuraminate transporter.

In an alternative and/or additional preferred embodiment, the cell comprises a pathway for production of a sialylated di- and/or oligosaccharide wherein the cell expresses an N-acetylneuraminate synthase like is known e,g. from N. meningitidis, Streptomyces sp., C. jejuni, which converts N-acetylmannosamine (ManNAc) into N-acetylneuraminate, an N-acylneuraminate cytidylyltransferase and a sialyltransferase as is described herein. ManNAc can be added to the cell and/or can be provided by an enzyme expressed in the cell or by the mechanism of the cell. Such cell producing ManNAc can express an UDP-N-acetylglucosamine 2-epimerase as described herein. Preferably, the cell is modified to produce ManNAc. More preferably, the cell is modified for enhanced production of ManNAc. The modification can be e.g., expression of an UDP-N-acetylglucosamine 2-epimerase.

In an alternative and/or additional preferred embodiment, the cell comprises a pathway for production of a sialylated di- and/or oligosaccharide wherein the cell expresses a phosphatase like e.g., the E. coli HAD-like phosphatase genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU, PsMupP from Pseudomonas putida, ScDOG1 from S. cerevisiae and BsAraL from Bacillus subtilis as described in WO 2018122225, which converts N-acetylglucosamine-6-phosphate (GlcNAc-6P) into N-acetylglucosamine (GlcNAc), and/or a phosphatase like e.g., an N-acylneuraminate-9-phosphatase from Candidatus Magnetomorum sp. HK-1 or Bacteroides thetaiotaomicron, which converts N-acylneuraminate-9-phosphate into sialic acid combined with e.g., expression of an N-acylglucosamine 2-epimerase, an N-acetylneuraminate synthase, an N-acylneuraminate cytidylyltransferase and a sialyltransferase as is described herein.

In an alternative and/or additional preferred embodiment, the cell comprises a pathway for production of a sialylated di- and/or oligosaccharide wherein the cell expresses an UDP-N-acetylglucosamine 2-epimerase like is known, e.g., from several species including C. jejuni, E. coli, N. meningitidis, Bacillus subtilis, Citrobacter rodentium, which converts UDP-N-acetylglucosamine (UDP-GlcNAc) into ManNAc, an N-acetylneuraminate synthase, and a sialyltransferase wherein the enzymes are as defined herein. UDP-GlcNAc can be added to the cell and/or can be provided by an enzyme expressed in the cell or by the metabolism of the cell. Such cell producing an UDP-GlcNAc is described herein. Preferably, the cell is modified to produce UDP-GlcNAc. More preferably, the cell is modified for enhanced UDP-GlcNAc production as described herein.

In an alternative and/or additional preferred embodiment, the cell comprises a pathway for production of a sialylated di- and/or oligosaccharide wherein the cell expresses an N-aceylneuraminate-9-phosphate synthetase, like is known e.g., from several species including Homo sapiens, Mus musculus, Rattus norvegicus, which converts N-acetylmannosamine-6-phosphate (ManNAc-6-P) into N-acetylneuraminate-9-phosphate, a phosphatase, an N-acylneuraminate cytidylyltransferase and a sialyltransferase as described herein. ManNAc-6-P can be added to the cell and/or can be provided by an enzyme expressed in the cell or by the metabolism of the cell. Such cell producing ManNAc-6-P can e.g., express a bifunctional UDP-GlcNAc 2-epimerase/kinase as described herein. Preferably, the cell is modified to produce ManNAc-6-P. More preferably, the cell is modified for enhanced ManNAc-6-P production.

In an alternative and/or additional preferred embodiment, the cell comprises a pathway for production of a sialylated di- and/or oligosaccharide wherein the cell expresses a bifunctional UDP-GlcNAc 2-epimerase/kinase like is known e.g., from several species including H. sapiens, R. norvegicus and Mus musculus, which converts UDP-GlcNAc into ManNAc-6-P an N-acylneuraminate-9-phosphate synthetase, an N-acylneuraminate-9-phosphate phosphatase, an N-acetylneuraminate synthase, an N-acylneuraminate cytidylyltransferase, and a sialyltransferase, wherein the enzymes are as defined herein. UDP-GlcNAc can be added to the cell and/or can be provided by an enzyme expressed in the cell or by the metabolism of the cell. Such cell producing an UDP-GlcNAc is described herein. Preferably, the cell is modified to produce UDP-GlcNAc. More preferably, the cell is modified for enhanced UDP-GlcNAc production as described herein.

In an alternative and/or additional preferred embodiment, the cell comprises a pathway for production of a sialylated di- and/or oligosaccharide wherein the cell expresses an N-acetylmannosamine-6-phosphate 2-epimerase like is known e.g., from several species including E. coli, Haemophilus influenzae, Enterobacter sp., Streptomyces sp., which converts N-acetylglucosamine-6-phosphate (GlcNAc-6P) into ManNAc-6-P, an N-acylneuraminate-9-phosphate synthetase, an N-acylneuraminate-9-phosphate phosphatase, an N-acetylneuraminate an N-acylneuraminate cytidylyltransferase and a sialyltransferase e, wherein the enzymes are as defined herein. GlcNAc-6P can be added to the cell and/or can be provided by an enzyme expressed in the cell or by the metabolism of the cell. Such cell producing GlcNAc-6P can express an enzyme converting, e.g., GlcN6P, which is to be added to the cell, to GlcNAc-6P. This enzyme may be a glucosamine 6-phosphate N-acetyltransferase from several species including Saccharomyces cerevisiae, Kluyveromyces lactis, Homo sapiens. Preferably, the cell is modified to produce GlcNAc-6P. More preferably, the cell is modified for enhanced GlcNAc-6P production. The modification can be any one or more chosen from the group comprising knockout of a glucosamine-6-phosphate deaminase, an N-acetylglucosamine-6-phosphate deacetylase and over-expression of an L-glutamine-D-fructose-6-phosphate aminotransferase and/or a glucosamine 6-phosphate N-acetyltransferase.

In an alternative and/or additional preferred embodiment, the cell comprises a pathway for production of a sialylated di- and/or oligosaccharide wherein the cell expresses an N-acylglucosamine 2-epimerase like is known e.g., from several species including Bacteroides ovatus, E. coli, H. sapiens, R. norvegicus, which converts GlcNAc into ManNAc, an N-acetylneuraminate synthase, an N-acylneuraminate cytidylyltransferase, and a sialyltransferase, wherein the enzymes are as defined herein. GlcNAc can be added to the cell and/or can be provided by an enzyme expressed in the cell or by the mechanism of the cell. Such cell producing GlcNAc can express a phosphatase converting GlcNAc-6-phosphate into GlcNAc, like any one or more of e.g., the E. coli HAD-like phosphatase genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU, PsMupP from Pseudomonas putida, ScDOG1 from S. cerevisiae and BsAraL from Bacillus subtilis as described in WO 2018122225. Preferably, the cell is modified to produce GlcNAc. More preferably, the cell is modified for enhanced GlcNAc production. The modification can be any one or more chosen from the group comprising knockout of a glucosamine-6-phosphate deaminase, an N-acetylglucosamine-6-phosphate deacetylase and/or an N-acetyl-D-glucosamine kinase and over-expression of an L-glutamine D-fructose-6-phosphate aminotransferase and/or a glucosamine 6-phosphate N-acetyltransferase.

Additionally, or alternatively, the host cell used herein is optionally genetically modified to import a precursor and/or an acceptor in the cell, by the introduction and/or overexpression of a membrane transporter protein that is able to import the respective precursor and/or acceptor in the cell. Such transporter is, for example, a membrane transporter protein belonging to the siderophore exporter family, the major facilitator superfamily (MFS), the ATP-binding cassette (ABC) transporter family, the sugar efflux transporter family or the PTS system involved in the uptake of e.g., mono-, di- and/or oligosaccharides.

Additionally, or alternatively, the host cell used herein is optionally genetically modified to import lactose in the cell, by the introduction and/or overexpression of a lactose permease. The lactose permease is, for example, encoded by the lacY gene or the lac12 gene.

Additionally, or alternatively, the host cell used herein is optionally genetically modified to export a sialylated di- and/or oligosaccharide over the membrane. Such transporter is, for example, a membrane transporter protein belonging to the siderophore exporter family, the major facilitator superfamily (MFS), the ATP-binding cassette (ABC) transporter family or the sugar efflux transporter family.

In a preferred embodiment of the method and/or cell of the disclosure, the cell comprises multiple coding DNA sequences wherein the multiple coding DNA sequences comprise multiple copies of the same coding DNA sequence that encode for one protein.

In another and/or additional preferred embodiment of the method and/or cell of the disclosure, the cell comprises multiple coding DNA sequences wherein the multiple coding DNA sequences comprise multiple different coding DNA sequence that encode for one protein.

In another and/or additional preferred embodiment of the method and/or cell of the disclosure, the cell comprises multiple coding DNA sequences wherein the multiple coding DNA sequences comprise multiple different coding DNA sequence that encode for multiple isoproteins that catalyze the same chemical reaction.

According to one aspect of the method and/or cell of the disclosure, the term “multiple” is at least two. In a preferred embodiment of the method and/or cell of the disclosure, the term “multiple” is at least three. In a more preferred embodiment of the method and/or cell of the disclosure, the term “multiple” is at least five.

In an exemplary embodiment of the method and/or cell of the disclosure, the cell comprises two identical coding DNA sequences that encode for the same protein. In another exemplary embodiment of the method and/or cell of the disclosure, the cell comprises two different coding DNA sequences that encode for the same protein. In another exemplary embodiment of the method and/or cell of the disclosure, the cell comprises two different coding DNA sequences that encode for two isoproteins that catalyze the same chemical reaction.

In another exemplary embodiment of the method and/or cell of the disclosure, the cell comprises three identical coding DNA sequences that encode for the same protein. In another exemplary embodiment of the method and/or cell of the disclosure, the cell comprises two identical coding DNA sequences and one coding DNA sequence that is different from the other two coding DNA sequences wherein the three coding DNA sequences encode for the same protein. In another exemplary embodiment of the method and/or cell of the disclosure, the cell comprises two identical coding DNA sequences and one coding DNA sequence that is different from the other two coding DNA sequences wherein the three coding DNA sequences encode for two isoproteins that catalyze the same chemical reaction. In another exemplary embodiment of the method and/or cell of the disclosure, the cell comprises three different coding DNA sequences that encode for the same protein. In another exemplary embodiment of the method and/or cell of the disclosure, the cell comprises three different coding DNA sequences that encode for three isoproteins that catalyze the same chemical reaction.

In another exemplary embodiment of the method and/or cell of the disclosure, the cell comprises five identical coding DNA sequences that encode for the same protein. In another exemplary embodiment of the method and/or cell of the disclosure, the cell comprises five different coding DNA sequences that encode for the same protein. In another exemplary embodiment of the method and/or cell of the disclosure, the cell comprises five different coding DNA sequences that encode for two isoproteins that catalyze the same chemical reaction. In another exemplary embodiment of the method and/or cell of the disclosure, the cell comprises five different coding DNA sequences that encode for three isoproteins that catalyze the same chemical reaction. In another exemplary embodiment of the method and/or cell of the disclosure, the cell comprises five different coding DNA sequences that encode for four isoproteins that catalyze the same chemical reaction. In another exemplary embodiment of the method and/or cell of the disclosure, the cell comprises five different coding DNA sequences that encode for five isoproteins that catalyze the same chemical reaction.

In a preferred aspect of the method and/or cell of the disclosure, the metabolically engineered cell is modified with one or more gene expression modules comprising multiple coding DNA sequences wherein the expression from any one of the multiple coding DNA sequences is regulated by one or more regulatory sequences. In another preferred aspect of the method and/or cell of the disclosure, the expression of any one or more of the expression modules is constitutive or is tuneable.

The expression modules are also known as transcriptional units and comprise polynucleotides for expression of recombinant genes including coding DNA sequences and appropriate transcriptional and/or translational control signals that are operably linked to the coding genes. The control signals comprise promoter sequences, untranslated regions, ribosome binding sites, terminator sequences. The expression modules can contain elements for expression of one single recombinant gene but can also contain elements for expression of more recombinant genes or can be organized in an operon structure for integrated expression of two or more recombinant genes. The polynucleotides may be produced by recombinant DNA technology using techniques well-known in the art. Methods that are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred aspect of the disclosure, the cell is modified with one or more expression modules. The expression modules can be integrated in the genome of the cell or can be presented to the cell on a vector. The vector can be present in the form of a plasmid, cosmid, phage, liposome, or virus, which is to be stably transformed/transfected into the metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the disclosure. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. The recombinant gene is involved in the expression of a polypeptide acting in the synthesis of the sialylated di- and/or oligosaccharide; or the recombinant gene is linked to other pathways in the host cell that are not involved in the synthesis of the sialylated di- and/or oligosaccharide. The recombinant genes encode endogenous proteins with a modified expression or activity, preferably the endogenous proteins are overexpressed; or the recombinant genes encode heterologous proteins that are heterogeneously introduced and expressed in the modified cell, preferably overexpressed. The endogenous proteins can have a modified expression in the cell, which also expresses a heterologous protein.

According to a preferred aspect of the disclosure, the expression of each of the expression modules is constitutive or tuneable as defined herein.

According to another aspect of the method and/or cell of the disclosure, the protein and/or isoproteins encoded by multiple coding DNA sequences is/are involved in the synthesis of a nucleotide-activated sugar. Herein, the nucleotide-activated sugar is to be used in the production of a sialylated di- and/or oligosaccharide. In a preferred embodiment of the method and/or cell of the disclosure, the nucleotide-activated sugar is chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-fucose (GDP-Fuc), GDP-rhamnose and UDP-xylose. In a more preferred embodiment of the method and/or cell of the disclosure, the nucleotide-activated sugar is chosen from the list comprising UDP-N-acetylglucosamine (UDP-GlcNAc), UDP-N-acetylmannosamine (UDP-ManNAc), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂ and CMP-N-glycolylneuraminic acid (CMP-Neu5Gc).

According to a preferred aspect of the method and/or cell of the disclosure, the protein and/or isoproteins encoded by multiple coding DNA sequences and that is/are involved in the synthesis of a nucleotide-activated sugar is/are chosen from the list comprising mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, N-acetylmannosamine-6-phosphate 2-epimerase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, CMP-sialic acid synthetase, galactose-1-epimerase, galactokinase, glucokinase, galactose-1-phosphate uridylyltransferase, UDP-glucose 4-epimerase, glucose-1-phosphate uridylyltransferase, glucophosphomutase, N-acetylglucosamine 1-phosphate uridylyltransferase, UDP-N-acetylglucosamine 4-epimerase, UDP-galactose 4-epimerase, N-acetylgalactosamine kinase, UDP-GalNAc pyrophosphorylase, mannose-1-phosphate guanyltransferase, UDP-GlcNAc 2-epimerase and ManNAc kinase.

In a further aspect of the method and/or cell of the disclosure, the cell is modified in the expression or activity of at least one of the protein and/or isoprotein involved in the synthesis of a nucleotide-activated sugar. In a preferred embodiment, the protein and/or isoprotein involved in the synthesis of a nucleotide-activated sugar is an endogenous protein of the cell with a modified expression or activity, preferably the endogenous protein and/or isoprotein involved in the synthesis of a nucleotide-activated sugar is overexpressed; alternatively the protein and/or isoprotein involved in the synthesis of a nucleotide-activated sugar is a heterologous protein that is heterogeneously introduced and expressed in the cell, preferably overexpressed. The endogenous protein and/or isoprotein involved in the synthesis of a nucleotide-activated sugar can have a modified expression in the cell, which also expresses a heterologous protein and/or isoprotein involved in the synthesis of a nucleotide-activated sugar.

In a preferred embodiment of the method and/or cell of disclosure, the cell is modified to produce UDP-GlcNAc from e.g., GlcNAc by expression of enzymes like e.g., an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. More preferably, the cell is modified for enhanced UDP-GlcNAc production. The modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

In another preferred embodiment of the method and/or cell of disclosure, the cell is modified to express de novo synthesis of CMP-sialic acid like e.g., CMP-Neu5Ac or CMP-Neu5Gc.

Such cell producing CMP-Neu5Ac can express an enzyme converting, e.g., sialic acid to CMP-Neu5Ac. This enzyme may be a CMP-sialic acid synthetase, like the N-acylneuraminate cytidylyltransferase from several species including Homo sapiens, Neisseria meningitidis, and Pasteurella multocida. More preferably, the cell is modified for enhanced CMP-Neu5Ac production. The modification can be any one or more chosen from the group comprising knock-out of an N-acetylglucosamine-6-phosphate deacetylase, knock-out of an glucosamine-6-phosphate deaminase, over-expression of a CMP-sialic acid synthetase, and over-expression of an N-acetyl-D-glucosamine-2-epimerase encoding gene.

CMP-Neu5Gc can be synthesized directly from CMP-Neu5Ac via a hydroxylation reaction performed by a vertebrate CMP-Neu5Ac hydroxylase (CMAH) enzyme. More preferably, the cell is modified for enhanced CMP-Neu5Gc production.

In another preferred embodiment of the method and/or cell of disclosure, the host cell used herein is optionally genetically modified to express the de novo synthesis of GDP-fucose. GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. The modification can be any one or more chosen from the group comprising knock-out of an UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

In another preferred embodiment of the method and/or cell of disclosure, the host cell used herein is optionally genetically modified to express the de novo synthesis of UDP-Gal. UDP-Gal can be provided by an enzyme expressed in the cell or by the metabolism of the cell. Such cell producing UDP-Gal can express an enzyme converting, e.g., UDP-glucose, to UDP-Gal. This enzyme may be, e.g., the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. The modification can be any one or more chosen from the group comprising knock-out of a bifunctional 5′-nucleotidase/UDP-sugar hydrolase encoding gene, knock-out of a galactose-1-phosphate uridylyltransferase encoding gene and over-expression of an UDP-glucose-4-epimerase encoding gene.

In another preferred embodiment of the method and/or cell of disclosure, the host cell used herein is optionally genetically modified to express the de novo synthesis of UDP-GalNAc. UDP-GalNAc can be synthesized from UDP-GlcNAc by the action of a single-step reaction using an UDP-N-acetylglucosamine 4-epimerase like e.g., wbgU from Plesiomonas shigelloides, gne from Yersinia enterocolitica or wbpP from Pseudomonas aeruginosa serotype 06. Preferably, the cell is modified to produce UDP-GalNAc. More preferably, the cell is modified for enhanced UDP-GalNAc production.

In another preferred embodiment of the method and/or cell of disclosure, the host cell used herein is optionally genetically modified to express the de novo synthesis of UDP-ManNAc. UDP-ManNAc can be synthesized directly from UDP-GlcNAc via an epimerization reaction performed by an UDP-GlcNAc 2-epimerase (like e.g., cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

According to another aspect of the method and/or cell of the disclosure, the cell expresses at least one glycosyltransferase chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

In a preferred embodiment of the method and/or cell of the disclosure, the fucosyltransferase is chosen from the list comprising alpha-1,2-fucosyltransferase, alpha-1,3-fucosyltransferase, alpha-1,4-fucosyltransferase and alpha-1,6-fucosyltransferase.

In an alternative and/or additional embodiment of the method and/or cell of the disclosure, the sialyltransferase is chosen from the list comprising alpha-2,3-sialyltransferase, alpha-2,6-sialyltransferase, and alpha-2,8-sialyltransferase.

In an alternative and/or additional embodiment of the method and/or cell of the disclosure, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase.

In an alternative and/or additional embodiment of the method and/or cell of the disclosure, the glucosyltransferase is chosen from the list comprising alpha-glucosyltransferase, beta-1,2-glucosyltransferase, beta-1,3-glucosyltransferase and beta-1,4-glucosyltransferase.

In an alternative and/or additional embodiment of the method and/or cell of the disclosure, the mannosyltransferase is chosen from the list comprising alpha-1,2-mannosyltransferase, alpha-1,3-mannosyltransferase and alpha-1,6-mannosyltransferase.

In an alternative and/or additional embodiment of the method and/or cell of the disclosure, the N-acetylglucosaminyltransferase is chosen from the list comprising galactoside beta-1,3-N-acetylglucosaminyltransferase and beta-1,6-N-acetylglucosaminyltransferase.

In an alternative and/or additional embodiment of the method and/or cell of the disclosure, the N-acetylgalactosaminyltransferase is chosen from the list comprising alpha-1,3-N-acetylgalactosaminyltransferase.

In a further aspect of the method and/or cell of the disclosure, the cell is modified in the expression or activity of at least one of the glycosyltransferases. In a preferred embodiment, the glycosyltransferase is an endogenous protein of the cell with a modified expression or activity, preferably the endogenous glycosyltransferase is overexpressed; alternatively the glycosyltransferase is a heterologous protein that is heterogeneously introduced and expressed in the cell, preferably overexpressed. The endogenous glycosyltransferase can have a modified expression in the cell, which also expresses a heterologous glycosyltransferase.

In an additional and/or alternative further aspect of the method and/or cell of the disclosure, the cell is modified for expression and/or overexpression of multiple coding DNA sequences that encode two or more glycosyltransferases that catalyze the same chemical reaction. In a preferred embodiment of the method and/or cell of the disclosure, the one or more glycosyltransferases expressed in the cell by multiple coding DNA sequences are enzymes with alpha-1,2-; alpha-1,3-; alpha-1,4- and/or alpha-1,6-fucosyltransferase activity.

In an alternative and/or additional embodiment of the method and/or cell of the disclosure, the one or more glycosyltransferases expressed in the cell by multiple coding DNA sequences are enzymes with alpha-2,3-; alpha-2,6- and/or alpha-2,8-sialyltransferase activity.

In an alternative and/or additional embodiment of the method and/or cell of the disclosure, the one or more glycosyltransferases expressed in the cell by multiple coding DNA sequences are enzymes with alpha-1,3-; alpha-1,4-; beta-1,3- and/or beta-1,4-galactosyltransferase activity.

In an alternative and/or additional embodiment of the method and/or cell of the disclosure, the one or more glycosyltransferases expressed in the cell by multiple coding DNA sequences are enzymes with N-acetylglucosamine beta-1,3- and/or N-acetylglucosamine beta-1,4-galactosyltransferase activity.

In an alternative and/or additional embodiment of the method and/or cell of the disclosure, the one or more glycosyltransferases expressed in the cell by multiple coding DNA sequences are enzymes with alpha-; beta-1,2-; beta-1,3- and/or beta-1,4-glucosyltransferase activity.

In an alternative and/or additional embodiment of the method and/or cell of the disclosure, the one or more glycosyltransferases expressed in the cell by multiple coding DNA sequences are enzymes with alpha-1,2-; alpha-1,3- and/or alpha-1,6-mannosyltransferase activity.

In an alternative and/or additional embodiment of the method and/or cell of the disclosure, the one or more glycosyltransferases expressed in the cell by multiple coding DNA sequences are enzymes with galactoside beta-1,3- and/or beta-1,6-N-acetylglucosaminyltransferase activity.

In an alternative and/or additional embodiment of the method and/or cell of the disclosure, the one or more glycosyltransferases expressed in the cell by multiple coding DNA sequences are enzymes with alpha-1,3-N-acetylgalactosaminyltransferase activity.

According to another aspect of the method and/or cell of the disclosure, the protein that is encoded by multiple coding DNA sequences is a membrane transporter protein. According to a further aspect of the method and/or cell of the disclosure, the membrane transporter protein is involved in the production of the sialylated di- and/or oligosaccharide.

According to a preferred embodiment of the method and/or cell of the disclosure, the cell expresses two or more copies of a membrane transporter protein that is chosen from the list comprising a siderophore exporter, an ABC transporter, an MFS transporter and a Sugar Efflux Transporter. In an exemplary embodiment of the method and/or cell of the disclosure, the cell comprises two or more coding DNA sequences that encode for the same siderophore exporter like e.g., the E. coli genes comprising entS with SEQ ID NO:49, MdfA with SEQ ID NO:50 and iceT with SEQ ID NO:51. In another and/or additional exemplary embodiment of the method and/or cell of the disclosure, the cell comprises two or more coding DNA sequences that encode for the same ABC transporter like e.g., oppF from E. coli with SEQ ID NO:52, lmrA from Lactococcus lactis subsp. lactis bv. diacetylactis and Blon_2475 from Bifidobacterium longum subsp. infantis. In another and/or additional exemplary embodiment of the method and/or cell of the disclosure, the cell comprises two or more coding DNA sequences that encode for the same MFS transporter like e.g., the E. coli genes comprising entS with SEQ ID NO:49, MdfA with SEQ ID NO:50 and iceT with SEQ ID NO:51. In another and/or additional exemplary embodiment of the method and/or cell of the disclosure, the cell comprises two or more coding DNA sequences that encode for the same Sugar Efflux Transporter like e.g., the E. coli genes comprising setA with SEQ ID NO:55, setB with SEQ ID NO:56 and setC with SEQ ID NO:57.

In another and/or additional exemplary embodiment of the method and/or cell of the disclosure, the cell comprises one coding DNA sequence of a siderophore exporter combined with two or more coding DNA sequences of any one or more of an ABC transporter, an MFS transporter and/or a Sugar Efflux Transporter. In another and/or additional exemplary embodiment of the method and/or cell of the disclosure, the cell comprises one coding DNA sequence of an ABC transporter combined with two or more coding DNA sequences of any one or more of a siderophore exporter, an MFS transporter and/or a Sugar Efflux Transporter. In another and/or additional exemplary embodiment of the method and/or cell of the disclosure, the cell comprises one coding DNA sequence of an MFS transporter combined with two or more coding DNA sequences of any one or more of a siderophore exporter, an ABC transporter and/or a Sugar Efflux Transporter. In another and/or additional exemplary embodiment of the method and/or cell of the disclosure, the cell comprises one coding DNA sequence of a Sugar Efflux Transporter combined with two or more coding DNA sequences of any one or more of a siderophore exporter, an ABC transporter and/or an MFS transporter.

In another and/or additional exemplary embodiment of the method and/or cell of the disclosure, the cell comprises one coding DNA sequence of a siderophore exporter and one coding DNA sequence of an ABC transporter combined with two or more coding DNA sequences of any one or more of an MFS transporter and/or a Sugar Efflux Transporter. In another and/or additional exemplary embodiment of the method and/or cell of the disclosure, the cell comprises one coding DNA sequence of a siderophore exporter and one coding DNA sequence of an MFS transporter combined with two or more coding DNA sequences of any one or more of an ABC transporter and/or a Sugar Efflux Transporter. In another and/or additional exemplary embodiment of the method and/or cell of the disclosure, the cell comprises one coding DNA sequence of a siderophore exporter and one coding DNA sequence of a Sugar Efflux Transporter combined with two or more coding DNA sequences of any one or more of an ABC transporter and/or an MFS transporter. In another and/or additional exemplary embodiment of the method and/or cell of the disclosure, the cell comprises one coding DNA sequence of an ABC transporter and one coding DNA sequence of an MFS transporter combined with two or more coding DNA sequences of any one or more of a siderophore exporter and/or a Sugar Efflux Transporter. In another and/or additional exemplary embodiment of the method and/or cell of the disclosure, the cell comprises one coding DNA sequence of an ABC transporter and one coding DNA sequence of a Sugar Efflux Transporter combined with two or more coding DNA sequences of any one or more of a siderophore exporter and/or an MFS transporter. In another and/or additional exemplary embodiment of the method and/or cell of the disclosure, the cell comprises one coding DNA sequence of an MFS transporter and one coding DNA sequence of a Sugar Efflux Transporter combined with two or more coding DNA sequences of any one or more of a siderophore exporter and/or an ABC transporter.

In another and/or additional exemplary embodiment of the method and/or cell of the disclosure, the cell comprises one coding DNA sequence of a siderophore exporter, one coding DNA sequence of an ABC transporter and one coding DNA sequence of an MFS transporter combined with two or more coding DNA sequences of a Sugar Efflux Transporter. In another and/or additional exemplary embodiment of the method and/or cell of the disclosure, the cell comprises one coding DNA sequence of a siderophore exporter, one coding DNA sequence of an MFS transporter and one coding DNA sequence of a Sugar Efflux Transporter combined with two or more coding DNA sequences of an ABC transporter. In another and/or additional exemplary embodiment of the method and/or cell of the disclosure, the cell comprises one coding DNA sequence of an ABC transporter, one coding DNA sequence of an MFS transporter and one coding DNA sequence of a Sugar Efflux Transporter combined with two or more coding DNA sequences of a siderophore exporter.

In a further aspect of the method and/or cell of the disclosure, the cell is modified in the expression or activity of at least one of the membrane transporter protein. In a preferred embodiment, the membrane transporter protein is an endogenous protein of the cell with a modified expression or activity, preferably the endogenous membrane transporter protein is overexpressed; alternatively the membrane transporter protein is a heterologous protein that is heterogeneously introduced and expressed in the cell, preferably overexpressed. The endogenous membrane transporter protein can have a modified expression in the cell, which also expresses a heterologous membrane transporter protein.

According to another aspect of the method and/or cell of the disclosure, the sialylated di- and/or oligosaccharide is chosen from the list comprising a milk oligosaccharide, O-antigen, enterobacterial common antigen (ECA), the oligosaccharide repeats present in capsular polysaccharides, peptidoglycan, amino-sugars and Lewis-type antigen oligosaccharides. In a preferred embodiment, the milk oligosaccharide is a mammalian milk oligosaccharide. In a more preferred embodiment, the milk oligosaccharide is a human milk oligosaccharide.

According to another aspect of the method and/or cell of the disclosure, the cell comprises a fucosylation pathway as described herein. According to a preferred embodiment, at least one protein encoding an enzyme as part of a fucosylation pathway is encoded by multiple coding DNA sequences that encode one or more enzymes that catalyze the same chemical reaction. According to a more preferred embodiment, at least one protein encoding an enzyme as part of a fucosylation pathway is encoded by two coding DNA sequences that encode one or more enzymes that catalyze the same chemical reaction. According to an even more preferred embodiment, at least one protein encoding an enzyme as part of a fucosylation pathway is encoded by three or more coding DNA sequences that encode one or more enzymes that catalyze the same chemical reaction.

According to another aspect of the method and/or cell of the disclosure, the cell comprises a galactosylation pathway as described herein. According to a preferred embodiment, at least one protein encoding an enzyme as part of a galactosylation pathway is encoded by multiple coding DNA sequences that encode one or more enzymes that catalyze the same chemical reaction. According to a more preferred embodiment, at least one protein encoding an enzyme as part of a galactosylation pathway is encoded by two coding DNA sequences that encode one or more enzymes that catalyze the same chemical reaction. According to an even more preferred embodiment, at least one protein encoding an enzyme as part of a galactosylation pathway is encoded by three or more coding DNA sequences that encode one or more enzymes that catalyze the same chemical reaction.

According to another aspect of the method and/or cell of the disclosure, the cell comprises an N-acetylglucosaminylation pathway as described herein. According to a preferred embodiment, at least one protein encoding an enzyme as part of an N-acetylglucosaminylation pathway is encoded by multiple coding DNA sequences that encode one or more enzymes that catalyze the same chemical reaction. According to a more preferred embodiment, at least one protein encoding an enzyme as part of an N-acetylglucosaminylation pathway is encoded by two coding DNA sequences that encode one or more enzymes that catalyze the same chemical reaction. According to an even more preferred embodiment, at least one protein encoding an enzyme as part of an N-acetylglucosaminylation pathway is encoded by three or more coding DNA sequences that encode one or more enzymes that catalyze the same chemical reaction.

According to another preferred aspect of the method and/or cell of the disclosure, the cell is capable to synthesize N-acetylmannosamine (ManNAc), N-acetylmannosamine-6-phosphate (ManNAc-6-phosphate) and/or phosphoenolpyruvate (PEP).

In a preferred embodiment, the cell comprises a pathway for production of a sialylated di- and/or oligosaccharide comprising a pathway for production of ManNAc. ManNAc can be provided by an enzyme expressed in the cell or by the mechanism of the cell. Such cell producing ManNAc can express an N-acylglucosamine 2-epimerase like is known e.g., from several species including Bacteroides ovatus, E. coli, Homo sapiens, Rattus norvegicus that converts GlcNAc into ManNAc. Alternatively, and/or additionally, the cell producing ManNAc can express an UDP-N-acetylglucosamine 2-epimerase like is known e.g., from several species including Campylobacter jejuni, E. coli, Neisseria meningitidis, Bacillus subtilis, Citrobacter rodentium that converts UDP-GlcNAc into ManNAc. GlcNAc and/or UDP-GlcNAc can be added to the cell and/or provided by an enzyme expressed in the cell or by the mechanism of the cell as described herein.

In a more preferred embodiment, the cell is modified for enhanced ManNAc production. The modification can be any one or more chosen from the group comprising knock-out of N-acetylmannosamine kinase, over-expression of N-acetylneuraminate lyase.

In another preferred embodiment, the cell comprises a pathway for production of a sialylated di- and/or oligosaccharide comprising a pathway for production of ManNAc-6-phosphate. ManNAc-6-phosphate can be provided by an enzyme expressed in the cell or by the mechanism of the cell. Such cell producing ManNAc-6-phosphate can express a bifunctional UDP-GlcNAc 2-epimerase/kinase like is known e.g., from several species including Homo sapiens, Rattus norvegicus and Mus musculus that converts UDP-GlcNAc into ManNAc-6-phosphate. Alternatively, and/or additionally, the cell producing ManNAc-6-phosphate can express an N-acetylmannosamine-6-phosphate 2-epimerase that converts GlcNAc-6-phosphate into ManNAc-6-phosphate. UDP-GlcNAc and/or GlcNAc-6-phosphate can be added to the cell and/or provided by an enzyme expressed in the cell or by the mechanism of the cell as described herein. In a more preferred embodiment, the cell is modified for enhanced ManNAc-6-phosphate production. The modification can be any one or more chosen from the group comprising over-expression of N-acetylglucosamine-6-phosphate deacetylase, over-expression of N-acetyl-D-glucosamine kinase, over-expression of phosphoglucosamine mutase, over-expression of N-acetylglucosamine-1-phosphate uridyltransferase/glucosamine-1-phosphate acetyltransferase.

In another preferred embodiment, the cell comprises a pathway for production of a sialylated di- and/or oligosaccharide comprising a pathway for production of phosphoenolpyruvate (PEP).

In another preferred embodiment, the cell comprises a pathway for production of a sialylated di- and/or oligosaccharide comprising any one or more modifications for enhanced production and/or supply of PEP.

In a preferred embodiment and as a means for enhanced production and/or supply of PEP, one or more PEP-dependent, sugar-transporting phosphotransferase system(s) is/are disrupted such as but not limited to: 1) the N-acetyl-D-glucosamine Npi-phosphotransferase (EC 2.7.1.193), which is, for instance, encoded by the nagE gene (or the cluster nagABCD) in E. coli or Bacillus species, 2) ManXYZ, which encodes the Enzyme 11 Man complex (mannose PTS permease, protein-Npi-phosphohistidine-D-mannose phosphotransferase) that imports exogenous hexoses (mannose, glucose, glucosamine, fructose, 2-deoxyglucose, mannosamine, N-acetylglucosamine, etc.) and releases the phosphate esters into the cell cytoplasm, 3) the glucose-specific PTS transporter (for instance, encoded by PtsG/Crr) which takes up glucose and forms glucose-6-phosphate in the cytoplasm, 4) the sucrose-specific PTS transporter, which takes up sucrose and forms sucrose-6-phosphate in the cytoplasm, 5) the fructose-specific PTS transporter (for instance, encoded by the genesfruA andfruB and the kinasefruK, which takes up fructose and forms in a first step fructose-1-phosphate and in a second step fructose1,6 bisphosphate, 6) the lactose PTS transporter (for instance, encoded by lacE in Lactococcus casei) which takes up lactose and forms lactose-6-phosphate, 7) the galactitol-specific PTS enzyme, which takes up galactitol and/or sorbitol and forms galactitol-1-phosphate or sorbitol-6-phosphate respectively, 8) the mannitol-specific PTS enzyme, which takes up mannitol and/or sorbitol and forms mannitol-1-phosphate or sorbitol-6-phosphate respectively, and 9) the trehalose-specific PTS enzyme, which takes up trehalose and forms trehalose-6-phosphate.

In another and/or additional preferred embodiment and as a means for enhanced production and/or supply of PEP, the full PTS system is disrupted by disrupting the PtsIH/Crr gene cluster. PtsI (Enzyme I) is a cytoplasmic protein that serves as the gateway for the phosphoenolpyruvate:sugar phosphotransferase system (PTS^{sugar}) of E. coli K-12. PtsI is one of two (PtsI and PtsH) sugar non-specific protein constituents of the PTS^{sugar}, which along with a sugar-specific inner membrane permease effects a phosphotransfer cascade that results in the coupled phosphorylation and transport of a variety of carbohydrate substrates. HPr (histidine containing protein) is one of two sugar-non-specific protein constituents of the PTS^{sugar}. It accepts a phosphoryl group from phosphorylated Enzyme I (PtsI-P) and then transfers it to the EIIA domain of any one of the many sugar-specific enzymes (collectively known as Enzymes II) of the PTS^{sugar}. Crr or EIIA^{Glc} is phosphorylated by PEP in a reaction requiring PtsH and PtsI.

In another and/or additional preferred embodiment, the cell is further modified to compensate for the deletion of a PTS system of a carbon source by the introduction and/or overexpression of the corresponding permease. These are e.g., permeases or ABC transporters that comprise but are not limited to transporters that specifically import lactose such as e.g., the transporter encoded by the LacY gene from E. coli, sucrose such as e.g., the transporter encoded by the cscB gene from E. coli, glucose such as e.g., the transporter encoded by the galP gene from E. coli, fructose such as e.g., the transporter encoded by the fruI gene from Streptococcus mutans, or the Sorbitol/mannitol ABC transporter such as the transporter encoded by the cluster SmoEFGK of Rhodobacter sphaeroides, the trehalose/sucrose/maltose transporter such as the transporter encoded by the gene cluster ThuEFGK of Sinorhizobium meliloti and the N-acetylglucosamine/galactose/glucose transporter such as the transporter encoded by NagP of Shewanella oneidensis. Examples of combinations of PTS deletions with overexpression of alternative transporters are: 1) the deletion of the glucose PTS system, e.g., ptsG gene, combined with the introduction and/or overexpression of a glucose permease (e.g., galP of glcP), 2) the deletion of the fructose PTS system, e.g., one or more of thefruB, fruA, fruK genes, combined with the introduction and/or overexpression of fructose permease, e.g., fruI, 3) the deletion of the lactose PTS system, combined with the introduction and/or overexpression of lactose permease, e.g., LacY, and/or 4) the deletion of the sucrose PTS system, combined with the introduction and/or overexpression of a sucrose permease, e.g., cscB.

In a further preferred embodiment, the cell is modified to compensate for the deletion of a PTS system of a carbon source by the introduction of carbohydrate kinases, such as glucokinase (EC 2.7.1.1, EC 2.7.1.2, EC 2.7.1.63), galactokinase (EC 2.7.1.6), and/or fructokinase (EC 2.7.1.3, EC 2.7.1.4). Examples of combinations of PTS deletions with overexpression of alternative transporters and a kinase are: 1) the deletion of the glucose PTS system, e.g., ptsG gene, combined with the introduction and/or overexpression of a glucose permease (e.g., galP of glcP), combined with the introduction and/or overexpression of a glucokinase (e.g., glk), and/or 2) the deletion of the fructose PTS system, e.g., one or more of thefruB, fruA, fruK genes, combined with the introduction and/or overexpression of fructose permease, e.g., fruI, combined with the introduction and/or overexpression of a fructokinase (e.g., frk or mak).

In another and/or additional preferred embodiment and as a means for enhanced production and/or supply of PEP, the cell is modified by the introduction of or modification in any one or more of the list comprising phosphoenolpyruvate synthase activity (EC: 2.7.9.2 encoded, for instance, in E. coli by ppsA), phosphoenolpyruvate carboxykinase activity (EC 4.1.1.32 or EC 4.1.1.49 encoded, for instance, in Corynebacterium glutamicum by PCK or in E. coli by pckA, resp.), phosphoenolpyruvate carboxylase activity (EC 4.1.1.31 encoded, for instance, in E. coli by ppc), oxaloacetate decarboxylase activity (EC 4.1.1.112 encoded, for instance, in E. coli by eda), pyruvate kinase activity (EC 2.7.1.40 encoded, for instance, in E. coli by pykA and pykF), pyruvate carboxylase activity (EC 6.4.1.1 encoded, for instance, in B. subtilis by pyc) and malate dehydrogenase activity (EC 1.1.1.38 or EC 1.1.1.40 encoded, for instance, in E. coli by maeA or maeB, resp.).

In a more preferred embodiment, the cell is modified to overexpress any one or more of the polypeptides comprising ppsA from E. coli with SEQ ID NO:41, PCK from C. glutamicum with SEQ ID NO:42, pcka from E. coli with SEQ ID NO:43, eda from E. coli with SEQ ID NO:44, maeA from E. coli with SEQ ID NO:45 and maeB from E. coli with SEQ ID NO:46.

In another and/or additional preferred embodiment, the cell is modified to express any one or more of a functional homolog, variant or derivative of any one of SEQ ID NO:41, 42, 43, 44, 45 or 46 having at least 80% overall sequence identity to the full-length of any one of the polypeptide with SEQ ID NOs:41, 42, 43, 44, 45 or 46, and having phosphoenolpyruvate synthase activity, phosphoenolpyruvate carboxykinase activity, oxaloacetate decarboxylase activity, or malate dehydrogenase activity, respectively.

In another and/or additional preferred embodiment and as a means for enhanced production and/or supply of PEP, the cell is modified by a reduced activity of phosphoenolpyruvate carboxylase activity, and/or pyruvate kinase activity, preferably a deletion of the genes encoding for phosphoenolpyruvate carboxylase, the pyruvate carboxylase activity and/or pyruvate kinase.

In an exemplary embodiment, the cell is genetically modified by different adaptations such as the overexpression of phosphoenolpyruvate synthase combined with the deletion of a pyruvate kinase gene, the overexpression of phosphoenolpyruvate synthase combined with the deletion of a phosphoenolpyruvate carboxylase gene, the overexpression of phosphoenolpyruvate synthase combined with the deletion of a pyruvate carboxylase gene, the overexpression of phosphoenolpyruvate carboxykinase combined with the deletion of a pyruvate kinase gene, the overexpression of phosphoenolpyruvate carboxykinase combined with the deletion of a phosphoenolpyruvate carboxylase gene, the overexpression of phosphoenolpyruvate carboxykinase combined with the deletion of a pyruvate carboxylase gene, the overexpression of oxaloacetate decarboxylase combined with the deletion of a pyruvate kinase gene, the overexpression of oxaloacetate decarboxylase combined with the deletion of a phosphoenolpyruvate carboxylase gene, the overexpression of oxaloacetate decarboxylase combined with the deletion of a pyruvate carboxylase gene, the overexpression of malate dehydrogenase combined with the deletion of a pyruvate kinase gene, the overexpression of malate dehydrogenase combined with the deletion of a phosphoenolpyruvate carboxylase gene and/or the overexpression of malate dehydrogenase combined with the deletion of a pyruvate carboxylase gene.

In another exemplary embodiment, the cell is genetically modified by different adaptations such as the overexpression of phosphoenolpyruvate synthase combined with the overexpression of a phosphoenolpyruvate carboxykinase, the overexpression of phosphoenolpyruvate synthase combined with the overexpression of an oxaloacetate decarboxylase, the overexpression of phosphoenolpyruvate synthase combined with the overexpression of a malate dehydrogenase, the overexpression of phosphoenolpyruvate carboxykinase combined with the overexpression of an oxaloacetate decarboxylase, the overexpression of phosphoenolpyruvate carboxykinase combined with the overexpression of a malate dehydrogenase, the overexpression of oxaloacetate decarboxylase combined with the overexpression of a malate dehydrogenase, the overexpression of phosphoenolpyruvate synthase combined with the overexpression of a phosphoenolpyruvate carboxykinase and the overexpression of an oxaloacetate decarboxylase, the overexpression of phosphoenolpyruvate synthase combined with the overexpression of a phosphoenolpyruvate carboxykinase and the overexpression of a malate dehydrogenase, the overexpression of phosphoenolpyruvate synthase combined with the overexpression of a phosphoenolpyruvate carboxykinase and the overexpression of an oxaloacetate decarboxylase and the overexpression of a malate dehydrogenase, the overexpression of phosphoenolpyruvate carboxykinase combined with the overexpression of an oxaloacetate decarboxylase and the overexpression of a malate dehydrogenase and/or the overexpression of phosphoenolpyruvate synthase combined with the overexpression of an oxaloacetate decarboxylase and the overexpression of a malate dehydrogenase.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene, the overexpression of phosphoenolpyruvate synthase combined with the overexpression of an oxaloacetate decarboxylase combined with the deletion of a pyruvate kinase gene, the overexpression of phosphoenolpyruvate synthase combined with the overexpression of a malate dehydrogenase combined with the deletion of a pyruvate kinase gene, the overexpression of phosphoenolpyruvate carboxykinase combined with the overexpression of an oxaloacetate decarboxylase combined with the deletion of a pyruvate kinase gene, the overexpression of phosphoenolpyruvate carboxykinase combined with the overexpression of a malate dehydrogenase combined with the deletion of a pyruvate kinase gene, the overexpression of an oxaloacetate decarboxylase combined with the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene, the overexpression of phosphoenolpyruvate synthase combined with the overexpression of a phosphoenolpyruvate carboxykinase and the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene, the overexpression of a phosphoenolpyruvate carboxykinase combined with the overexpression of an oxaloacetate decarboxylase and the overexpression of a malate dehydrogenase combined with the deletion of a pyruvate kinase gene, the overexpression of phosphoenolpyruvate synthase combined the overexpression of oxaloacetate decarboxylase and the overexpression of a malate dehydrogenase combined with the deletion of a pyruvate kinase gene.

In another exemplary embodiment, the cell is genetically modified by different adaptations such as the overexpression of phosphoenolpyruvate synthase combined with the overexpression of a phosphoenolpyruvate carboxykinase combined with the deletion of a phosphoenolpyruvate carboxylase gene, the overexpression of phosphoenolpyruvate synthase combined with the overexpression of an oxaloacetate decarboxylase combined with the deletion of a phosphoenolpyruvate carboxylase gene, the overexpression of phosphoenolpyruvate synthase combined with the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase combined with the deletion of a phosphoenolpyruvate carboxylase gene, the overexpression of a phosphoenolpyruvate carboxykinase combined with the overexpression of a malate dehydrogenase combined with the deletion of a phosphoenolpyruvate carboxylase gene, the overexpression of an oxaloacetate decarboxylase combined with the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a phosphoenolpyruvate carboxylase gene, the overexpression of phosphoenolpyruvate synthase combined the overexpression of an oxaloacetate decarboxylase and the overexpression of a malate dehydrogenase combined with the deletion of a phosphoenolpyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate carboxylase gene, the overexpression of phosphoenolpyruvate synthase combined with the overexpression of an oxaloacetate decarboxylase combined with the deletion of a pyruvate carboxylase gene, the overexpression of phosphoenolpyruvate synthase combined with the overexpression of a malate dehydrogenase combined with the deletion of a pyruvate carboxylase gene, the overexpression of a phosphoenolpyruvate carboxykinase combined with the overexpression of an oxaloacetate decarboxylase combined with the deletion of a pyruvate carboxylase gene, the overexpression of a phosphoenolpyruvate carboxykinase combined with the overexpression of a malate dehydrogenase combined with the deletion of a pyruvate carboxylase gene, the overexpression of an oxaloacetate decarboxylase combined with the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate carboxylase gene, the overexpression of phosphoenolpyruvate synthase combined with the overexpression of an oxaloacetate decarboxylase and the overexpression of a malate dehydrogenase combined with the deletion of a pyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene and a phosphoenolpyruvate carboxylase gene, the overexpression of phosphoenolpyruvate synthase combined with the overexpression of an oxaloacetate decarboxylase combined with the deletion of a pyruvate kinase gene and a phosphoenolpyruvate carboxylase gene, the overexpression of phosphoenolpyruvate synthase combined with the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a phosphoenolpyruvate carboxylase gene, the overexpression of a phosphoenolpyruvate carboxykinase combined with the overexpression of a malate dehydrogenase combined with the deletion of a pyruvate kinase gene and a phosphoenolpyruvate carboxylase gene, the overexpression of an oxaloacetate decarboxylase combined with the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate synthase combined with the overexpression of an oxaloacetate decarboxylase and the overexpression of a malate dehydrogenase combined with the deletion of a pyruvate kinase gene and a phosphoenolpyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a malate dehydrogenase combined with the deletion of a pyruvate kinase gene and a pyruvate carboxylase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a pyruvate carboxylase gene and a phosphoenolpyruvate carboxylase gene, the overexpression of a phosphoenolpyruvate carboxykinase combined with the overexpression of a malate dehydrogenase combined with the deletion of a pyruvate kinase gene and a pyruvate carboxylase gene and a phosphoenolpyruvate carboxylase gene, the overexpression of an oxaloacetate decarboxylase combined with the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and pyruvate carboxylase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene.

According to another aspect of the method and/or cell of the disclosure, the cell comprises at least one coding DNA sequence encoding a protein having N-acetylneuraminate synthase activity, two or more coding DNA sequences encoding 2 or more isoproteins having N-acylneuraminate cytidylyltransferase activity and two or more copies of one or more coding DNA sequences of an alpha-2,3-sialyltransferase, an alpha-2,6-sialyltransferase, and/or an alpha-2,8-sialyltransferase. In a preferred embodiment of the method and/or cell of the disclosure, the protein having N-acetylneuraminate synthase activity is the N-acetylneuraminate synthase from N. meningitidis (NmNeuB) with SEQ ID NO:01. In an alternative preferred embodiment of the method and/or cell of the disclosure, the protein having N-acetylneuraminate synthase activity is a functional homolog or functional fragment of the N-acetylneuraminate synthase from N. meningitidis (NmNeuB) with SEQ ID NO:01. In another alternative preferred embodiment of the method and/or cell of the disclosure, the protein having N-acetylneuraminate synthase activity is a polypeptide sequence having at least 80% sequence identity to the full-length sequence of the N-acetylneuraminate synthase from N. meningitidis (NmNeuB) with SEQ ID NO:01 and having N-acetylneuraminate synthase activity. In another preferred embodiment of the method and/or cell of the disclosure, the isoproteins having N-acylneuraminate cytidylyltransferase activity are chosen from the list comprising the protein from Campylobacter jejuni (CjNeuA) with SEQ ID NO:02, Helicobacter influenzae (HiNeuA) with SEQ ID NO:03 and Pasteurella multocida (PmultNeuA) with SEQ ID NO:04. In an alternative preferred embodiment of the method and/or cell of the disclosure, the isoproteins having N-acylneuraminate cytidylyltransferase activity are a functional homolog or functional fragment of any one of the proteins with SEQ ID NOs:02, 03 or 04. In another alternative preferred embodiment of the method and/or cell of the disclosure, the isoproteins having N-acylneuraminate cytidylyltransferase activity are a polypeptide sequence having at least 80% sequence identity to the full-length sequence of any one of the proteins with SEQ ID NO:02, 03 or 04, respectively, and having N-acylneuraminate cytidylyltransferase activity.

According to another aspect of the method and/or cell of the disclosure, the cell comprises two or more copies of a coding DNA sequence encoding an enzyme having L-glutamine-D-fructose-6-phosphate aminotransferase activity. In a preferred embodiment of the method and/or cell, the cell comprises two or more copies of a coding DNA sequence encoding the enzyme from E. coli (glmS*54) with SEQ ID NO:05. In another and/or additional preferred embodiment of the method and/or cell, the cell comprises two or more copies of a coding DNA sequence encoding a functional homolog or functional fragment of the L-glutamine-D-fructose-6-phosphate aminotransferase from E. coli (glmS*54) with SEQ ID NO:05. In another and/or additional preferred embodiment of the method and/or cell, the cell comprises two or more copies of a coding DNA sequence encoding a polypeptide sequence having at least 80% sequence identity to the full-length sequence of the L-glutamine-D-fructose-6-phosphate aminotransferase from E. coli (glmS*54) with SEQ ID NO:05 and having L-glutamine D-fructose-6-phosphate aminotransferase activity.

According to another and/or additional aspect of the method and/or cell of the disclosure, the cell comprises two or more copies of a coding DNA sequence encoding an enzyme having glucosamine 6-phosphate N-acetyltransferase activity. In a preferred embodiment of the method and/or cell, the cell comprises two or more copies of a coding DNA sequence encoding the enzyme from Saccharomyces cerevisiae (GNA1) with SEQ ID NO:06. In another and/or additional preferred embodiment of the method and/or cell, the cell comprises two or more copies of a coding DNA sequence encoding a functional homolog or functional fragment of the glucosamine 6-phosphate N-acetyltransferase from S. cerevisiae (GNA1) with SEQ ID NO:06. In another and/or additional preferred embodiment of the method and/or cell, the cell comprises two or more copies of a coding DNA sequence encoding a polypeptide sequence having at least 80% sequence identity to the full-length sequence of the glucosamine 6-phosphate N-acetyltransferase from S. cerevisiae (GNA1) with SEQ ID NO:06 and having glucosamine 6-phosphate N-acetyltransferase activity.

According to another preferred aspect of the method and/or cell of the disclosure, the cell comprises a modification for reduced production of acetate. The modification can be any one or more chosen from the group comprising overexpression of an acetyl-coenzyme A synthetase, a full or partial knock-out or rendered less functional pyruvate dehydrogenase and a full or partial knock-out or rendered less functional lactate dehydrogenase.

In a further aspect of the method and/or cell of the disclosure, the cell is modified in the expression or activity of at least one acetyl-coenzyme A synthetase like e.g., acs from E. coli, S. cerevisiae, H. sapiens, M. musculus. In a preferred embodiment, the acetyl-coenzyme A synthetase is an endogenous protein of the cell with a modified expression or activity, preferably the endogenous acetyl-coenzyme A synthetase is overexpressed; alternatively, the acetyl-coenzyme A synthetase is a heterologous protein that is heterogeneously introduced and expressed in the cell, preferably overexpressed. The endogenous acetyl-coenzyme A synthetase can have a modified expression in the cell, which also expresses a heterologous can have a modified expression in the cell, which also expresses a heterologous. In a more preferred embodiment, the cell is modified in the expression or activity of the acetyl-coenzyme A synthetase acs from E. coli with SEQ ID NO:47. In another and/or additional preferred embodiment, the cell is modified in the expression or activity of a functional homolog, variant or derivative of SEQ ID NO:47 having at least 80% overall sequence identity to the full-length of the polypeptide with SEQ ID NO:47 and having acetyl-coenzyme A synthetase activity.

In an alternative and/or additional further aspect of the method and/or cell of the disclosure, the cell is modified in the expression or activity of at least one pyruvate dehydrogenase like e.g., from E. coli, S. cerevisiae, H. sapiens and R. norvegicus. In a preferred embodiment, the cell has been modified to have at least one partially or fully knocked out or mutated pyruvate dehydrogenase encoding gene by means generally known by the person skilled in the art resulting in at least one protein with less functional or being disabled for pyruvate dehydrogenase activity. In a more preferred embodiment, the cell has a full knock-out in the poxB encoding gene resulting in a cell lacking pyruvate dehydrogenase activity.

In an alternative and/or additional further aspect of the method and/or cell of the disclosure, the cell is modified in the expression or activity of at least one lactate dehydrogenase like e.g., from E. coli, S. cerevisiae, H. sapiens and R. norvegicus. In a preferred embodiment, the cell has been modified to have at least one partially or fully knocked out or mutated lactate dehydrogenase encoding gene by means generally known by the person skilled in the art resulting in at least one protein with less functional or being disabled for lactate dehydrogenase activity. In a more preferred embodiment, the cell has a full knock-out in the ldhA encoding gene resulting in a cell lacking lactate dehydrogenase activity.

According to another preferred aspect of the method and/or cell of the disclosure, the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose undecaprenyl-phosphate glucose-1-phosphatetransferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

According to another preferred aspect of the method and/or cell of the disclosure, the cell comprises a catabolic pathway for selected mono-, di- or oligosaccharides, which is at least partially inactivated, the mono-, di-, or oligosaccharides being involved in and/or required for the synthesis of a sialylated di- and/or oligosaccharide.

According to another preferred aspect of the method and/or cell of the disclosure, the cell is using a precursor for the synthesis of a sialylated di- and/or oligosaccharide. Herein, the precursor is fed to the cell from the cultivation medium. In another preferred embodiment, the cell is producing a precursor for the synthesis of the sialylated di- and/or oligosaccharide.

According to another preferred aspect of the method and/or cell of the disclosure, the cell produces 90 g/L or more of a sialylated di- and/or oligosaccharide in the whole broth and/or supernatant. In a more preferred embodiment, the sialylated di- and/or oligosaccharide produced in the whole broth and/or supernatant has a purity of at least 80% measured on the total amount of sialylated di- and/or oligosaccharide and its precursor produced by the cell in the whole broth and/or supernatant, respectively.

Another aspect of the disclosure provides for a method and a cell wherein a sialylated di- and/or oligosaccharide is produced in and/or by a bacterial, fungal, yeast, insect, plant, animal or protozoan expression system or cell as described herein. The expression system or cell is chosen from the list comprising a bacterium, a fungus, or a yeast, or, refers to a plant, animal, or protozoan cell. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably the disclosure specifically relates to a mutated and/or transformed Escherichia coli strain as indicated above wherein the E. coli strain is a K12 strain. More specifically, the disclosure relates to a mutated and/or transformed Escherichia coli strain as indicated above wherein the K12 strain is E. coli MG1655. The latter bacterium belonging to the phylum Firmicutes belongs preferably to the Bacilli, preferably from the species Bacillus. The latter fungus belongs preferably to the genus Rhizopus, Dictyostelium, Penicillium, Mucor or Aspergillus. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Zygosaccharomyces, Pichia, Komagataella, Hansenula, Yarrowia, Starmerella, Kluyveromyces or Debaromyces. Plant cells include cells of flowering and non-flowering plants, as well as algal cells, for example, Chlamydomonas, Chlorella, etc. Preferably, the plant is a tobacco, alfalfa, rice, tomato, cotton, rapeseed, soy, maize or corn plant. The latter animal cell is preferably derived from non-human mammals (e.g., cattle, buffalo, pig, sheep, mouse, rat), birds (e.g., chicken, duck, ostrich, turkey, pheasant), fish (e.g., swordfish, salmon, tuna, sea bass, trout, catfish), invertebrates (e.g., lobster, crab, shrimp, clams, oyster, mussel, sea urchin), reptiles (e.g., snake, alligator, turtle), amphibians (e.g., frogs) or insects (e.g., fly, nematode) or is a genetically modified cell line derived from human cells excluding embryonic stem cells. Both human and non-human mammalian cells are preferably chosen from the list comprising an epithelial cell like e.g., a mammary epithelial cell, an embryonic kidney cell, a fibroblast cell, a COS cell, a Chinese hamster ovary (CHO) cell, a murine myeloma cell like e.g., an N20, SP2/0 or YB2/0 cell, an NIH-3T3 cell, a non-mammary adult stem cell or derivatives thereof such as described in WO 2021067641. The latter insect cell is preferably derived from Spodoptera frugiperda like e.g., Sf9 or Sf21 cells, Bombyx mori, Mamestra brassicae, Trichoplusia ni like e.g., BTI-TN-5B1-4 cells or Drosophila melanogaster like e.g., Drosophila S2 cells. The latter protozoan cell preferably is a Leishmania tarentolae cell.

In a preferred embodiment of the method and/or cell of the disclosure, the cell is a viable Gram-negative bacterium that comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose compared to a non-modified progenitor.

In a more preferred embodiment of the method and/or cell, the reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose is provided by a mutation in any one or more glycosyltransferases involved in the synthesis of any one of the poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose, wherein the mutation provides for a deletion or lower expression of any one of the glycosyltransferases. The glycosyltransferases comprise glycosyltransferase genes encoding poly-N-acetyl-D-glucosamine synthase subunits, UDP-N-acetylglucosamine-undecaprenyl-phosphate N-acetylglucosaminephosphotransferase, Fuc4NAc (4-acetamido-4,6-dideoxy-D-galactose) transferase, UDP-N-acetyl-D-mannosaminuronic acid transferase, the glycosyltransferase genes encoding the cellulose synthase catalytic subunits, the cellulose biosynthesis protein, colanic acid biosynthesis glucuronosyltransferase, colanic acid biosynthesis galactosyltransferase, colanic acid biosynthesis fucosyltransferase, UDP-glucose:undecaprenyl-phosphate glucose-1-phosphate transferase, putative colanic biosynthesis glycosyl transferase, UDP-glucuronate:LPS(HepIII) glycosyltransferase, ADP-heptose-LPS heptosyltransferase 2, ADP-heptose:LPS heptosyltransferase 1, putative ADP-heptose:LPS heptosyltransferase 4, lipopolysaccharide core biosynthesis protein, UDP-glucose:(glucosyl)LPS α-1,2-glucosyltransferase, UDP-D-glucose:(glucosyl)LPS α-1,3-glucosyltransferase, UDP-D-galactose:(glucosyl)lipopolysaccharide-1,6-D-galactosyltransferase, lipopolysaccharide glucosyltransferase I, lipopolysaccharide core heptosyltransferase 3,3-1,6-galactofuranosyltransferase, undecaprenyl-phosphate 4-deoxy-4-formamido-L-arabinose transferase, lipid IVA 4-amino-4-deoxy-L-arabinosyltransferase, bactoprenol glucosyl transferase, putative family 2 glycosyltransferase, the osmoregulated periplasmic glucans (OPG) biosynthesis protein G, OPG biosynthesis protein H, glucosylglycerate phosphorylase, glycogen synthase, 1,4-α-glucan branching enzyme, 4-α-glucanotransferase and trehalose-6-phosphate synthase. In an exemplary embodiment, the cell is mutated in any one or more of the glycosyltransferases comprising pgaC, pgaD, rfe, rffT, rffM, bcsA, bcsB, bcsC, wcaA, wcaC, wcaE, wcaI, wcaJ, wcaL, waaH, waaF, waaC, waaU, waaZ, waaJ, waaO, waaB, waaS, waaG, waaQ, wbbl, arnC, arnT, yfdH, wbbK, opgG, opgH, ycjM, glgA, glgB, malQ, otsA and yaiP, wherein the mutation provides for a deletion or lower expression of any one of the glycosyltransferases.

In an alternative and/or additional preferred embodiment of the method and/or cell, the reduced or abolished synthesis of poly-N-acetyl-glucosamine (PNAG) is provided by over-expression of a carbon storage regulator encoding gene, deletion of a Na+/H+antiporter regulator encoding gene and/or deletion of the sensor histidine kinase encoding gene.

Another aspect provides for a cell to be stably cultured in a medium, wherein the medium can be any type of growth medium comprising minimal medium, complex medium or growth medium enriched in certain compounds, for example, but not limited to vitamins, trace elements, amino acids.

The cell as used herein is capable to grow on a monosaccharide, disaccharide, oligosaccharide, polysaccharide, polyol, glycerol, a complex medium or a mixture thereof as the main carbon source. With the term main is meant the most important carbon source for the cell for the production of the sialylated di- and/or oligosaccharide of interest, biomass formation, carbon dioxide and/or by-products formation (such as acids and/or alcohols, such as acetate, lactate, and/or ethanol), i.e., 20, 30, 40, 50, 60, 70, 75, 80, 85, 90, 95, 98, 99% of all the required carbon is derived from the above-indicated carbon source. In one embodiment of the disclosure, the carbon source is the sole carbon source for the organism, i.e., 100% of all the required carbon is derived from the above-indicated carbon source. Common main carbon sources comprise but are not limited to glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. As used herein, a precursor as defined herein cannot be used as a carbon source for the production of the sialylated di- and/or oligosaccharide.

According to another aspect of the method and/or cell of the disclosure, the cell is capable to synthesize a mixture of oligosaccharides comprising at least one sialylated oligosaccharide. In an alternative and/or additional aspect, the cell is capable to synthesize a mixture of di- and oligosaccharides comprising at least one sialylated di- and/or oligosaccharide; alternatively, the cell is capable to synthesize a mixture of sialic acid, di- and/or oligosaccharides.

In a further preferred aspect, the method for the production of a sialylated di- and/or oligosaccharide as described herein comprises at least one of the following steps:
i) Adding to the culture medium in a reactor at least one precursor and/or acceptor feed wherein the total reactor volume ranges from 250 mL (millilitre) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of the precursor and/or acceptor feed;
ii) Adding at least one precursor and/or acceptor feed in a continuous manner to the culture medium over the course of 1 day, 2 days, 3 days, 4 days, 5 days by means of a feeding solution;
iii) Adding at least one precursor and/or acceptor feed in a continuous manner to the culture medium over the course of 1 day, 2 days, 3 days, 4 days, 5 days by means of a feeding solution and wherein preferably, the pH of the feeding solution is set between 3 and 7 and wherein preferably, the temperature of the feeding solution is kept between 20° C. and 80° C.;
the method resulting in a sialylated di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

In another and/or additional further preferred aspect, the method for the production of a sialylated di- and/or oligosaccharide as described herein comprises at least one of the following steps:
i) Adding to the culture medium at least one precursor and/or acceptor in one pulse or in a discontinuous (pulsed) manner wherein the total reactor volume ranges from 250 mL (millilitre) to 10,000 m³ (cubic meter), preferably so that the final volume of the culture medium is not more than three-fold, preferably not more than two-fold, more preferably less than 2-fold of the volume of the culture medium before the addition of the precursor and/or acceptor feed pulse(s);
ii) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution;
iii) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution and wherein preferably, the pH of the feeding solution is set between 3 and 7 and wherein preferably, the temperature of the feeding solution is kept between 20° C. and 80° C.;
the method resulting in a sialylated di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

In a further, more preferred aspect, the method for the production of a sialylated di- and/or oligosaccharide as described herein comprises at least one of the following steps:
i) Adding to the culture medium a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of the lactose feed;
ii) Adding a lactose feed in a continuous manner to the culture medium over the course of 1 day, 2 days, 3 days, 4 days, 5 days by means of a feeding solution;
iii) Adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7 and wherein preferably the temperature of the feed solution is kept between 20° C. and 80° C.;
the method resulting in a sialylated oligosaccharide produced from the lactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

Preferably the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration>300 mM.

In another aspect the lactose feed is accomplished by adding lactose to the cultivation medium in a concentration, such that throughout the production phase of the cultivation a lactose concentration of at least 5 mM, preferably 10 mM or 30 mM is obtained.

In a further embodiment of the methods described herein the host cells are cultivated for at least about 60, 80, 100, or about 120 hours or in a continuous manner.

In a preferred embodiment, a carbon source is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per litre of initial culture volume in a continuous manner, so that the final volume of the culture medium is not more than three-fold, advantageously not more than two-fold, more advantageously less than two-fold of the volume of the culturing medium before the culturing.

Preferably, when performing the method as described herein, a first phase of exponential cell growth is provided by adding a carbon source, preferably glucose or sucrose, to the culture medium before the lactose is added to the culture medium in a second phase.

In an alternative preferable embodiment, in the method as described herein, the lactose is added already in the first phase of exponential growth together with the carbon-based substrate.

According to the disclosure, the methods as described herein preferably comprises a step of separating the sialylated di- and/or oligosaccharide from the cultivation.

The terms “separating from the cultivation” means harvesting, collecting, or retrieving the sialylated di- and/or oligosaccharide from the cell and/or the medium of its growth.

The sialylated di- and/or oligosaccharide can be separated in a conventional manner from the aqueous culture medium, in which the cell was grown. In case the sialylated di- and/or oligosaccharide is still present in the cells producing the sialylated di- and/or oligosaccharide, conventional manners to free or to extract the sialylated di- and/or oligosaccharide out of the cells can be used, such as cell destruction using high pH, heat shock, sonication, French press, homogenization, enzymatic hydrolysis, chemical hydrolysis, solvent hydrolysis, detergent, hydrolysis, . . . . The culture medium and/or cell extract together and separately can then be further used for separating the sialylated di- and/or oligosaccharide.

This preferably involves clarifying the sialylated di- and/or oligosaccharide to remove suspended particulates and contaminants, particularly cells, cell components, insoluble metabolites and debris produced by culturing the genetically modified cell. In this step, the sialylated di- and/or oligosaccharide can be clarified in a conventional manner. Preferably, the sialylated di- and/or oligosaccharide is clarified by centrifugation, flocculation, decantation and/or filtration. A second step of separating the sialylated di- and/or oligosaccharide preferably involves removing substantially all the proteins, peptides, amino acids, RNA and DNA, and any endotoxins and glycolipids that could interfere with the subsequent separation step, from the sialylated di- and/or oligosaccharide, preferably after it has been clarified. In this step, proteins and related impurities can be removed from the sialylated di- and/or oligosaccharide in a conventional manner. Preferably, proteins, salts, by-products, color, endotoxins and other related impurities are removed from the sialylated di- and/or oligosaccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g., using slab-polyacrylamide or sodium dodecyl sulphate-polyacrylamide gel electrophoresis (PAGE)), affinity chromatography (using affinity ligands including e.g., DEAE-SEPHAROSE®, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, remaining proteins and related impurities are retained by a chromatography medium or a selected membrane.

In a further preferred embodiment, the methods as described herein also provide for a further purification of the sialylated di- and/or oligosaccharide of disclosure. A further purification of the sialylated di- and/or oligosaccharide may be accomplished, for example, by use of (activated) charcoal or carbon, nanofiltration, ultrafiltration, electrophoresis, enzymatic treatment or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the sialylated di- and/or oligosaccharide. Another purification step is to dry, e.g., spray dry, lyophilize, spray freeze dry, freeze spray dry, band dry, belt dry, vacuum band dry, vacuum belt dry, drum dry, roller dry, vacuum drum dry or vacuum roller dry the produced sialylated di- and/or oligosaccharide.

In an exemplary embodiment, the separation and purification of the sialylated di- and/or oligosaccharide is made in a process, comprising the following steps in any order:
a) contacting the cultivation or a clarified version thereof with a nanofiltration membrane with a molecular weight cut-off (MWCO) of 600-3500 Da ensuring the retention of the produced sialylated di- and/or oligosaccharide and allowing at least a part of the proteins, salts, by-products, color and other related impurities to pass,
b) conducting a diafiltration process on the retentate from step a), using the membrane, with an aqueous solution of an inorganic electrolyte, followed by optional diafiltration with pure water to remove excess of the electrolyte,
c) and collecting the retentate enriched in the sialylated di- and/or oligosaccharide in the form of a salt from the cation of the electrolyte.

In an alternative exemplary embodiment, the separation and purification of the sialylated di- and/or oligosaccharide is made in a process, comprising the following steps in any order: subjecting the cultivation or a clarified version thereof to two membrane filtration steps using different membranes, wherein
one membrane has a molecular weight cut-off of between about 300 Dalton to about 500 Dalton, and
the other membrane as a molecular weight cut-off of between about 600 Dalton to about 800 Dalton.

In an alternative exemplary embodiment, the separation and purification of the sialylated di- and/or oligosaccharide is made in a process, comprising the following steps in any order comprising the step of treating the cultivation or a clarified version thereof with a strong cation exchange resin in H+-form and a weak anion exchange resin in free base form.

In an alternative exemplary embodiment, the separation and purification of the sialylated di- and/or oligosaccharide is made in the following way. The cultivation comprising the produced sialylated di- and/or oligosaccharide, biomass, medium components and contaminants is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,
wherein a purified solution comprising the produced sialylated di- and/or oligosaccharide at a purity of greater than or equal to 80 percent is provided. Optionally the purified solution is dried by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying.

In an alternative exemplary embodiment, the separation and purification of the sialylated di- and/or oligosaccharide is made in a process, comprising the following steps in any order: enzymatic treatment of the cultivation; removal of the biomass from the cultivation; ultrafiltration; nanofiltration; and a column chromatography step. Preferably such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, II and IV with different flow rates; and/or iii) an eluent comprising water; and/or iv) an operating temperature of 15 degrees to 60 degrees centigrade.

In a specific embodiment, the disclosure provides the produced sialylated di- and/or oligosaccharide, which is dried to powder by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying, wherein the dried powder contains <15 percent-wt. of water, preferably <10 percent-wt. of water, more preferably <7 percent-wt. of water, most preferably <5 percent-wt. of water.

Another aspect of the disclosure provides the use of a cell as defined herein, in a method for the production of a sialylated di- and/or oligosaccharide. A further aspect of the disclosure provides the use of a method as defined herein for the production of a sialylated di- and/or oligosaccharide.

Furthermore, the disclosure also relates to the sialylated di- and/or oligosaccharide obtained by the methods according to the disclosure, as well as to the use of a polynucleotide, the vector, host cells or the polypeptide as described above for the production of the sialylated di- and/or oligosaccharide. The sialylated di- and/or oligosaccharide may be used as food additive, prebiotic, symbiotic, for the supplementation of baby food, adult food or feed, or as either therapeutically or pharmaceutically active compound or in cosmetic applications. With the novel methods, the sialylated di- and/or oligosaccharide can easily and effectively be provided, without the need for complicated, time and cost consuming synthetic processes.

For identification of the sialylated di- and/or oligosaccharide produced in the cell as described herein, the monomeric building blocks (e.g., the monosaccharide or glycan unit composition), the anomeric configuration of side chains, the presence and location of substituent groups, degree of polymerization/molecular weight and the linkage pattern can be identified by standard methods known in the art, such as, e.g., methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the sialylated di- and/or oligosaccharide methods such as e.g., acid-catalyzed hydrolysis, HPLC (high performance liquid chromatography) or GLC (gas-liquid chromatography) (after conversion to alditol acetates) may be used. To determine the glycosidic linkages, the sialylated di- and/or oligosaccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the sialylated di- and/or oligosaccharide is subjected to enzymatic analysis, e.g., it is contacted with an enzyme that is specific for a particular type of linkage, e.g., beta-galactosidase, or alpha-glucosidase, etc., and NMR may be used to analyze the products.

The separated and preferably also purified sialylated di- and/or oligosaccharide as described herein is incorporated into a food (e.g., human food or feed), dietary supplement, pharmaceutical ingredient, cosmetic ingredient or medicine. In some embodiments, the sialylated di- and/or oligosaccharide is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the sialylated di- and/or oligosaccharide being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, a sialylated di- and/or oligosaccharide produced and/or purified by a process of this specification is orally administered in combination with such microorganism.

Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavorings.

In some embodiments, the sialylated oligosaccharide is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, a sialylated oligosaccharide produced and/or purified by a process in this specification is included in infant formula to provide nutritional benefits similar to those provided by the oligosaccharides in human breast milk. In some embodiments, the sialylated oligosaccharide is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, B6, B12, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the concentration of the sialylated oligosaccharide in the infant formula is approximately the same concentration as the concentration of the sialylated oligosaccharide generally present in human breast milk.

In some embodiments, the sialylated oligosaccharide is incorporated into a feed preparation, wherein the feed is chosen from the list comprising pet food, animal milk replacer, veterinary product, post weaning feed, or creep feed.

As will be shown in the examples herein, the method and the cell of the disclosure preferably provide at least one of the following surprising advantages:
Higher titers of the sialylated di- and/or oligosaccharide (g/L),
Higher production rate r (g sialylated di- and/or oligosaccharide/L/h),
Higher cell performance index CPI (g sialylated di- and/or oligosaccharide/g X),
Higher specific productivity Qp (g sialylated di- and/or oligosaccharide/g X/h),
Higher yield on sucrose Ys (g sialylated di- and/or oligosaccharide/g sucrose),
Higher sucrose uptake/conversion rate Qs (g sucrose/g X/h),
Higher lactose conversion/consumption rate rs (g lactose/h),
Higher secretion of the sialylated di- and/or oligosaccharide, and/or
Higher growth speed of the production host,
when compared to a host for production of a sialylated di- and/or oligosaccharide lacking expression and/or overexpression of multiple coding DNA sequences encoding one or more proteins that catalyze the same chemical reaction.

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features that are still to be explained below can be used not only in the respectively specified combinations, but also in other combinations or on their own, without departing from the scope of the disclosure.

Moreover, the disclosure relates to the following specific embodiments:

1. A metabolically engineered cell for production of a sialylated di- and/or oligosaccharide, the cell comprising a pathway for production of the sialylated di- and/or oligosaccharide, characterized in that the cell is modified for expression and/or overexpression of multiple coding DNA sequences encoding one or more proteins that catalyze the same chemical reaction.

2. Cell according to embodiment 1, wherein the protein is involved in the pathway for production of the sialylated di- and/or oligosaccharide.

3. Cell according to any one of embodiment 1 or 2, wherein the pathway for production of the sialylated di- and/or oligosaccharide comprises a sialylation pathway.

4. Cell according to embodiment 3, wherein the sialylation pathway comprises at least one protein chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-aceylneuraminate-9-phosphate synthetase, phosphatase, N-acetylneuraminate synthase, N-acylneuraminate cytidylyltransferase, sialyltransferase and sialic acid transporter,

preferably, wherein at least one of the proteins is encoded by the multiple coding DNA sequences.

5. Cell according to any one of the previous embodiments, wherein the multiple coding DNA sequences comprise any one or more of
multiple copies of the same coding DNA sequence encoding for one protein,
multiple coding DNA sequences encoding for one protein, and
multiple coding DNA sequences encoding for multiple isoproteins that catalyze the same chemical reaction.

6. Cell according to any one of the previous embodiments, wherein multiple is at least 2, preferably at least 3, more preferably at least 5.

7. Cell according to any one of the previous embodiments, wherein the coding DNA sequences are presented to the cell in one or more gene expression modules wherein expression is regulated by one or more regulatory sequences.

8. Cell according to embodiment 7, wherein the expression modules are integrated in the host cell's genome and/or presented to the cell on a vector comprising plasmid, cosmid, phage, liposome or virus, which is to be stably transformed into the host cell.

9. Cell according to any one of the previous embodiments, wherein the protein is involved in the synthesis of a nucleotide-activated sugar, wherein the nucleotide-activated sugar is to be used in the production of the sialylated di- and/or oligosaccharide.

10. Cell according to embodiment 9, wherein the nucleotide-activated sugar is chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N3, CMP-Neu4,5Ac2, CMP-Neu5,7Ac2, CMP-Neu5,9Ac2, CMP-Neu5,7(8,9)Ac2, CMP-N-glycolylneuraminic acid (CMP-Neu5Gc), GDP-fucose (GDP-Fuc), GDP-rhamnose and UDP-xylose,
preferably the nucleotide-activated sugar is chosen from the list comprising UDP-N-acetylglucosamine (UDP-GlcNAc), UDP-N-acetylmannosamine (UDP-ManNAc), CMP-sialic acid (CMP-Neu5Ac), CMP-Neu4Ac, CMP-Neu5Ac9N3, CMP-Neu4,5Ac2, CMP-Neu5,7Ac2, CMP-Neu5,9Ac2, CMP-Neu5,7(8,9)Ac2 and CMP-N-glycolylneuraminic acid (CMP-Neu5Gc).

11. Cell according to any one of embodiment 9 or 10, wherein the protein involved in the synthesis of a nucleotide-activated sugar is chosen from the list comprising mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, N-acetylmannosamine-6-phosphate 2-epimerase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, CMP-sialic acid synthetase, galactose-1-epimerase, galactokinase, glucokinase, galactose-1-phosphate uridylyltransferase, UDP-glucose 4-epimerase, glucose-1-phosphate uridylyltransferase, glucophosphomutase, N-acetylglucosamine 1-phosphate uridylyltransferase, UDP-N-acetylglucosamine 4-epimerase, UDP-galactose 4-epimerase, N-acetylgalactosamine kinase, UDP-GalNAc pyrophosphorylase, mannose-1-phosphate guanyltransferase, UDP-GlcNAc 2-epimerase and ManNAc kinase.

12. Cell according to any one of the previous embodiments, wherein the cell further expresses at least one glycosyltransferase chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
preferably, wherein the cell is modified in the expression or activity of at least one of the glycosyltransferases,
preferably, the fucosyltransferase is chosen from the list comprising alpha-1,2-fucosyltransferase, alpha-1,3-fucosyltransferase, alpha-1,4-fucosyltransferase and alpha-1,6-fucosyltransferase,
preferably, the sialyltransferase is chosen from the list comprising alpha-2,3-sialyltransferase, alpha-2,6-sialyltransferase, and alpha-2,8-sialyltransferase,
preferably, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
preferably, the glucosyltransferase is chosen from the list comprising alpha-glucosyltransferase, beta-1,2-glucosyltransferase, beta-1,3-glucosyltransferase and beta-1,4-glucosyltransferase,
preferably, the mannosyltransferase is chosen from the list comprising alpha-1,2-mannosyltransferase, alpha-1,3-mannosyltransferase and alpha-1,6-mannosyltransferase,
preferably, the N-acetylglucosaminyltransferase is chosen from the list comprising galactoside beta-1,3-N-acetylglucosaminyltransferase and beta-1,6-N-acetylglucosaminyltransferase,
preferably, the N-acetylgalactosaminyltransferase is chosen from the list comprising alpha-1,3-N-acetylgalactosaminyltransferase.

13. Cell according to any one of the previous embodiments, wherein the protein is a membrane transporter protein that is chosen from the list comprising a siderophore exporter, an ABC transporter, an MFS transporter and Sugar Efflux Transporter.

14. Cell according to any one of the previous embodiments, wherein the sialylated di- and/or oligosaccharide is chosen from the list comprising a milk oligosaccharide, O-antigen, enterobacterial common antigen (ECA), the oligosaccharide repeats present in capsular polysaccharides, peptidoglycan, amino-sugars and Lewis-type antigen oligosaccharides, preferably the milk oligosaccharide is a mammalian milk oligosaccharide, more preferably the milk oligosaccharide is a human milk oligosaccharide.

15. Cell according to any one of the previous embodiments, wherein the cell comprises a fucosylation pathway comprising at least one protein chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, fucosyltransferase,
preferably, wherein at least one of the proteins is encoded by the multiple coding DNA sequences encoding one or more enzymes that catalyze the same chemical reaction, wherein multiple is preferably two, more preferably three or more.

16. Cell according to any one of the previous embodiments, wherein the cell comprises a galactosylation pathway comprising at least one protein chosen from the list comprising galactose-1-epimerase, galactokinase, glucokinase, galactose-1-phosphate uridylyltransferase, UDP-glucose 4-epimerase, glucose-1-phosphate uridylyltransferase, glucophosphomutase, galactosyltransferase,
preferably, wherein at least one of the proteins is encoded by the multiple coding DNA sequences encoding one or more enzymes that catalyze the same chemical reaction, wherein multiple is preferably two, more preferably three or more.

17. Cell according to any one of the previous embodiments, wherein the cell comprises an N-acetylglucosaminylation pathway comprising at least one protein chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, N-acetylglucosaminyltransferase, preferably, wherein at least one of the proteins is encoded by the multiple coding DNA sequences encoding one or more enzymes that catalyze the same chemical reaction, wherein multiple is preferably two, more preferably three or more.

18. Cell according to any one of the previous embodiments, wherein the cell is modified for enhanced synthesis and/or supply of phosphoenolpyruvate (PEP).

19. Cell according to any one of the previous embodiments, wherein the cell comprises:
at least one coding DNA sequence encoding a protein chosen from the list comprising i) the enzyme from Neisseria meningitidis (NmNeuB) with SEQ ID NO:01 and having N-acetylneuraminate synthase activity, ii) a functional homolog or functional fragment of the enzyme with SEQ ID NO:01, and iii) a polypeptide sequence having at least 80% sequence identity to the full-length sequence of the enzyme with SEQ ID NO:01 and having N-acetylneuraminate synthase activity,
two or more coding DNA sequences encoding a protein chosen from the list comprising i) the enzyme from Campylobacter jejuni (CjNeuA) with SEQ ID NO:02, Helicobacter influenzae (HiNeuA) with SEQ ID NO:03 and Pasteurella multocida (PmultNeuA) with SEQ ID NO:04, wherein the enzymes with SEQ ID NOs:02, 03 and 04 have N-acylneuraminate cytidylyltransferase activity, ii) a functional homolog or functional fragment of any one of the enzymes with SEQ ID NOs:02, 03 or 04, and iii) a polypeptide sequence having at least 80% sequence identity to the full-length sequence of any one of the enzymes with SEQ ID NO:02, 03 or 04, respectively, and having N-acylneuraminate cytidylyltransferase activity, and
two or more copies of one or more coding DNA sequences of an alpha-2,3-sialyltransferase, an alpha-2,6-sialyltransferase, and/or an alpha-2,8-sialyltransferase.

20. Cell according to any one of the previous embodiments, wherein the cell comprises:
two or more copies of a coding DNA sequence encoding an enzyme having L-glutamine-D-fructose-6-phosphate aminotransferase activity and preferably chosen from the list comprising i) the enzyme from Escherichia coli (glmS*54) with SEQ ID NO:05 and ii) a functional homolog or functional fragment of the enzyme with SEQ ID NO:05, and iii) a polypeptide sequence having at least 80% sequence identity to the full-length sequence of the enzyme with SEQ ID NO:05 and having L-glutamine D-fructose-6-phosphate aminotransferase activity, and/or
two or more copies of a coding DNA sequence encoding an enzyme having glucosamine 6-phosphate N-acetyltransferase activity, preferably chosen from the list comprising i) the enzyme from Saccharomyces cerevisiae (GNA1) with SEQ ID NO:06, ii) a functional homolog or functional fragment of the enzyme with SEQ ID NO:06, and iii) a polypeptide sequence having at least 80% sequence identity to the full-length sequence of the enzyme with SEQ ID NO:06 and having glucosamine 6-phosphate N-acetyltransferase activity.

21. Cell according to any one of the previous embodiments, wherein the cell comprises a modification for reduced production of acetate.

22. Cell according to any one of the previous embodiments, wherein the cell further comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose undecaprenyl-phosphate glucose-1-phosphatetransferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

23. Cell according to any one of the previous embodiments, wherein the cell comprises a catabolic pathway for selected mono-, di- or oligosaccharides, which is at least partially inactivated, the mono-, di-, or oligosaccharides being involved in and/or required for the synthesis of the sialylated di- and/or oligosaccharide.

24. Cell according to any one of the previous embodiments, wherein the cell is using a precursor for the synthesis of the sialylated di- and/or oligosaccharide the precursor being fed to the cell from the cultivation medium.

25. Cell according to any one of the previous embodiments, wherein the cell is producing a precursor for the synthesis of the sialylated di- and/or oligosaccharide.

26. Cell according to any one of the previous embodiments, wherein the cell produces 90 g/L or more of the sialylated di- and/or oligosaccharide in the whole broth and/or supernatant and/or wherein the sialylated di- and/or oligosaccharide in the whole broth and/or supernatant has a purity of at least 80% measured on the total amount of sialylated di- and/or oligosaccharide and its precursor produced by the cell in the whole broth and/or supernatant, respectively.

27. Cell according to any one of the previous embodiments, wherein the cell is a bacterium, fungus, yeast, a plant cell, an animal cell, or a protozoan cell,
preferably the bacterium is of an Escherichia coli strain, more preferably of an Escherichia coli strain, which is a K-12 strain, even more preferably the Escherichia coli K-12 strain is E. coli MG1655,
preferably the fungus belongs to a genus chosen from the group comprising Rhizopus, Dictyostelium, Penicillium, Mucor or Aspergillus,
preferably the yeast belongs to a genus chosen from the group comprising Saccharomyces, Zygosaccharomyces, Pichia, Komagataella, Hansenula, Yarrowia, Starmerella, Kluyveromyces or Debaromyces,
preferably the plant cell is an algal cell or is derived from tobacco, alfalfa, rice, tomato, cotton, rapeseed, soy, maize, or corn plant,
preferably the animal cell is derived from non-human mammals, birds, fish, invertebrates, reptiles, amphibians or insects or is a genetically modified cell line derived from human cells excluding embryonic stem cells, more preferably the human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably the insect cell is derived from Spodoptera frugiperda, Bombyx mori, Mamestra brassicae, Trichoplusia ni or Drosophila melanogaster,
preferably the protozoan cell is a Leishmania tarentolae cell.

28. Cell according to embodiment 27, wherein the cell is a viable Gram-negative bacterium that comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose compared to a non-modified progenitor.

29. Cell according to any one of the previous embodiments, wherein the cell is stably cultured in a medium.

30. Cell according to any one of the previous embodiments, wherein the cell is capable to synthesize a mixture of oligosaccharides comprising at least one sialylated oligosaccharide.

31. Cell according to any one of the previous embodiments, wherein the cell is capable to synthesize a mixture of di- and oligosaccharides comprising at least one sialylated di- and/or oligosaccharide.

32. Method to produce a sialylated di- and/or oligosaccharide by a cell, the method comprising the steps of:
i) providing a cell according to any one of embodiments 1 to 31, and
ii) cultivating the cell under conditions permissive to produce the sialylated di- and/or oligosaccharide,
iii) preferably, separating the sialylated di- and/or oligosaccharide from the cultivation.

33. Method according to embodiment 32, the method further comprising at least one of the following steps:
i) Adding to the culture medium in a reactor at least one precursor and/or acceptor feed wherein the total reactor volume ranges from 250 mL (milliliter) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of the precursor and/or acceptor feed;
ii) Adding at least one precursor and/or acceptor feed in a continuous manner to the culture medium over the course of 1 day, 2 days, 3 days, 4 days, 5 days by means of a feeding solution;
iii) Adding at least one precursor and/or acceptor feed in a continuous manner to the culture medium over the course of 1 day, 2 days, 3 days, 4 days, 5 days by means of a feeding solution and wherein preferably, the pH of the feeding solution is set between 3 and 7 and wherein preferably, the temperature of the feeding solution is kept between 20° C. and 80° C.;
the method resulting in a sialylated di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

34. Method according to embodiment 32, the method further comprising at least one of the following steps:
i) adding to the culture medium a lactose feed comprising at least 50, more preferably at least 75, more preferably at least 100, more preferably at least 120, more preferably at least 150 gram of lactose per liter of initial reactor volume wherein the reactor volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of the lactose feed;
ii) adding a lactose feed in a continuous manner to the culture medium over the course of 1 day, 2 days, 3 days, 4 days, 5 days by means of a feeding solution;
iii) adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7 and wherein preferably the temperature of the feed solution is kept between 20° C. and 80° C.;
the method resulting in a sialylated oligosaccharide produced from the lactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

35. Method according to embodiment 34, wherein the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration>300 mM.

36. Method according to any one of embodiment 34 or 35, wherein the lactose feed is accomplished by adding lactose to the cultivation medium in a concentration, such, that throughout the production phase of the cultivation a lactose concentration of at least 5 mM, preferably 10 mM or 30 mM is obtained.

37. Method according to any one of embodiment 32 to 36, wherein the host cells are cultivated for at least about 60, 80, 100, or about 120 hours or in a continuous manner.

38. Method according to any one of embodiment 32 to 37, wherein the cell is cultivated in culture medium comprising a carbon source comprising a monosaccharide, disaccharide, oligosaccharide, polysaccharide, polyol, glycerol, a complex medium including molasses, corn steep liquor, peptone, tryptone or yeast extract; preferably, wherein the carbon source is chosen from the list comprising glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate.

39. Method according to any one of embodiment 32 to 38, wherein the cell uses at least one precursor for the synthesis of the sialylated di- and/or oligosaccharide, preferably the cell uses two or more precursors for the synthesis of the sialylated di- and/or oligosaccharide.

40. Method according to any one of embodiment 32 to 39, wherein the culture medium contains at least one compound selected from the group comprising lactose, galactose, sialic acid, fucose, GlcNAc, GalNAc, lacto-N-biose (LNB), N-acetyllactosamine (LacNAc).

41. Method according to any one of embodiment 32 to 40, wherein a first phase of exponential cell growth is provided by adding a carbon-based substrate, preferably glucose or sucrose, to the culture medium before the lactose is added to the culture medium in a second phase.

42. Method according to any one of embodiment 32 to 41, wherein the cell is producing at least one precursor for the synthesis of the sialylated di- and/or oligosaccharide.

43. Method according to any one of embodiment 32 to 42, wherein the precursor for the synthesis of the sialylated di- and/or oligosaccharide is completely converted into the sialylated di- and/or oligosaccharide.

44. Method according to any one of embodiment 32 to 43, wherein the sialylated di- and/or oligosaccharide is separated from the culture medium and/or the cell.

45. Method according to any one of embodiment 32 to 44, wherein the separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis, affinity chromatography, ion exchange chromatography, hydrophobic interaction chromatography and/or gel filtration, ligand exchange chromatography.

46. Method according to any one of embodiment 32 to 45, wherein the method further comprises purification of the sialylated di- and/or oligosaccharide.

47. Method according to embodiment 46, wherein the purification comprises at least one of the following steps: use of activated charcoal or carbon, use of charcoal, nanofiltration, ultrafiltration, electrophoresis, enzymatic treatment or ion exchange, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.

48. Use of a cell according to any one of embodiment 1 to 29 for production of a sialylated di- and/or oligosaccharide.

49. Use of a cell according to embodiment 30 for production of a mixture of oligosaccharides comprising at least one sialylated oligosaccharide.

50. Use of a cell according to embodiment 31 for production of a mixture of di- and oligosaccharides comprising at least one sialylated di- and/or oligosaccharide.

51. Use of a method according to any one of embodiment 32 to 47 for production of a sialylated di- and/or oligosaccharide.

The disclosure will be described in more detail in the examples. The following examples will serve as further illustration and clarification of the disclosure and are not intended to be limiting.

### EXAMPLES

### Example 1. Calculation of Percentage Identity Between Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48: 443-453) to find the global (i.e., spanning the full-length sequences) alignment of two sequences that maximizes the number of matches and minimizes the number of gaps. The BLAST algorithm (Altschul et al., J. Mol. Biol. (1990) 215: 403-10) calculates the global percentage sequence identity (i.e., over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity ((i.e., spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith T F, Waterman M S (1981) J. Mol. Biol 147(1); 195-7).

### Example 2. Materials and Methods Escherichia coli

Media

The Luria Broth (LB) medium consisted of 1% tryptone peptone (Difco, Erembodegem, Belgium), 0.5% yeast extract (Difco) and 0.5% sodium chloride (VWR, Leuven, Belgium). The minimal medium used in the cultivation experiments in 96-well plates or in shake flasks contained 2.00 g/L NH₄Cl, 5.00 g/L (NH₄)₂SO₄, 2.993 g/L KH₂PO₄, 7.315 g/L K₂HPO₄, 8.372 g/L MOPS, 0.5 g/L NaCl, 0.5 g/L MgSO₄·7H₂O, 30 g/L sucrose or 30 g/L glycerol, 1 ml/L vitamin solution, 100 μl/L molybdate solution, and 1 mL/L selenium solution. As specified in the respective examples, 0.30 g/L sialic acid, 20 g/L lactose, 20 g/L LacNAc and/or 20 g/L LNB were additionally added to the medium as precursor(s). The minimal medium was set to a pH of 7 with 1M KOH. Vitamin solution consisted of 3.6 g/L FeCl₂·4H₂O, 5 g/L CaCl₂)·2H₂O, 1.3 g/L MnCl₂·2H₂O, 0.38 g/L CuCl₂·2H₂O, 0.5 g/L COCl₂·6H₂O, 0.94 g/L ZnCl₂, 0.0311 g/L H₃BO₄, 0.4 g/L Na₂EDTA·2H₂O and 1.01 g/L thiamine·HCl. The molybdate solution contained 0.967 g/L NaMoO₄·2H₂O. The selenium solution contained 42 g/L SeO₂.

The minimal medium for fermentations contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄·7H₂O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 20 g/L lactose, 20 g/L LacNAc and/or 20 g/L LNB were additionally added to the medium as precursor(s).

Complex medium was sterilized by autoclaving (121° C., 21 min) and minimal medium by filtration (0.22 μm Sartorius). When necessary, the medium was made selective by adding an antibiotic: e.g., chloramphenicol (20 mg/L), carbenicillin (100 mg/L), spectinomycin (40 mg/L) and/or kanamycin (50 mg/L).

Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F⁻, phi80dlacZΔM15, Δ(IacZYA-argF) U169, deoR, recA1, endA1, hsdR17(rk⁻, mk⁺), phoA, supE44, lambda⁻, thi-1, gyrA96, relA1) bought from Invitrogen.

Strains and Mutations

Escherichia coli K12 MG1655 [λ⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain. Transformants carrying a Red helper plasmid pKD46 were grown in 10 mL LB media with ampicillin, (100 mg/L) and L-arabinose (10 mM) at 30° C. to an OD₆₀₀ nm of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a GENE PULSER™ (BioRad) (600 Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, re-purified from an agarose gel, and suspended in elution buffer (5 mM Tris, pH 8.0). Selected mutants were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers.

In an example for sialic acid production, the mutant strain was derived from E. coli K12 MG1655 comprising genomic knock-ins of constitutive transcriptional units containing one or more copies of a glucosamine 6-phosphate N-acetyltransferase like e.g., GNA1 from Saccharomyces cerevisiae with SEQ ID NO:7, an N-acetylglucosamine 2-epimerase like e.g., AGE from Bacteroides ovatus with SEQ ID NO:9 and one or more copies of an N-acetylneuraminate synthase like e.g., NeuB from Neisseria meningitidis with SEQ ID NO:01 or from Campylobacter jejuni with SEQ ID NO:02.

Alternatively and/or additionally, sialic acid production can be obtained by genomic knock-ins of constitutive transcriptional units containing an UDP-N-acetylglucosamine 2-epimerase like e.g., NeuC from C. jejuni with SEQ ID NO:12 and one or more copies of an N-acetylneuraminate synthase like e.g., NeuB from N. meningitidis with SEQ ID NO:01 or from C. jejuni with SEQ ID NO:02.

Alternatively and/or additionally, sialic acid production can be obtained by genomic knock-ins of constitutive transcriptional units containing a phosphoglucosamine mutase like e.g., glmM from E. coli with SEQ ID NO:10, an N-acetylglucosamine-1-phosphate uridyltransferase/glucosamine-1-phosphate acetyltransferase like e.g., glmU from E. coli with SEQ ID NO:11, an UDP-N-acetylglucosamine 2-epimerase like e.g., NeuC from C. jejuni with SEQ ID NO: 12 and one or more copies of an N-acetylneuraminate synthase like e.g., NeuB from N. meningitidis with SEQ ID NO:01 or from C. jejuni with SEQ ID NO:02.

Alternatively and/or additionally, sialic acid production can be obtained by genomic knock-ins of constitutive transcriptional units containing a bifunctional UDP-GlcNAc 2-epimerase/N-acetylmannosamine kinase like e.g., from Mus musculus (strain C57BL/6J) with SEQ ID NO:13, an N-acylneuraminate-9-phosphate synthetase like e.g., from Syntrophorhabdus sp. PtaU1.Bin058 with SEQ ID NO:14 and an N-acylneuraminate-9-phosphatase like e.g., from Candidatus Magnetomorum sp. HK-1 with SEQ ID NO:15 and/or from Bacteroides thetaiotaomicron (strain ATCC 29148) with SEQ ID NO:16.

Alternatively and/or additionally, sialic acid production can be obtained by genomic knock-ins of constitutive transcriptional units containing a phosphoglucosamine mutase like e.g., glmM from E. coli with SEQ ID NO:10, an N-acetylglucosamine-1-phosphate uridyltransferase/glucosamine-1-phosphate acetyltransferase like e.g., glmU from E. coli with SEQ ID NO:11, a bifunctional UDP-GlcNAc 2-epimerase/N-acetylmannosamine kinase like e.g., from M. musculus (strain C57BL/6J) with SEQ ID NO:13, an N-acylneuraminate-9-phosphate synthetase like e.g., from Syntrophorhabdus sp. PtaU1.Bin058 with SEQ ID NO:14 and an N-acylneuraminate-9-phosphatase like e.g., from Candidatus magnetomorum sp. HK-1 with SEQ ID NO:15 and/or from Bacteroides thetaiotaomicron (strain ATCC 29148) with SEQ ID NO:16.

Sialic acid production can further be optimized in the mutant E. coli strain with genomic knock-outs of the E. coli genes comprising any one or more of nagA, nagB, nagC, nagD, nagE, nanA, nanE, nanK, manX, manY and manZ as described in WO 2018122225, and/or genomic knock-outs of the E. coli genes comprising any one or more of nanT, poxB, 1dhA, adhE, aldB, pflA, pflC, ybiY, ackA and/or pta and with genomic knock-ins of constitutive transcriptional units comprising any one or more of one or more copies of a sialic acid transporter like e.g., nanT from E. coli with SEQ ID NO:08, one or more copies of a membrane transporter protein like e.g., entS from E. coli with SEQ ID NO:49, MdfA from E. coli with SEQ ID NO:50, iceT from E. coli with SEQ ID NO:51, oppF from E. coli with SEQ ID NO:52, lmrA from Lactococcus lactis subsp. lactis bv. diacetylactis with SEQ ID NO:53, Blon_2475 from Bifidobacterium longum subsp. infantis with SEQ ID NO:54, SetA from E. coli with SEQ ID NO:55, SetB from E. coli with SEQ ID NO:56 and SetC from E. coli with SEQ ID NO:57 or any combination thereof, one or more copies of an L-glutamine-D-fructose-6-phosphate aminotransferase like e.g., the mutant glmS*54 from E. coli with SEQ ID NO:06 (differing from the wild-type E. coli glmS by an A39T, an R250C and an G472S mutation), preferably a phosphatase like any one or more of e.g., the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae and BsAraL from Bacillus subtilis as described in WO 2018122225 and the acetyl-CoA synthetase acs from E. coli with SEQ ID NO:47.

For sialylated oligosaccharide production, the sialic acid production strains were further modified to express two or more isoproteins with N-acylneuraminate cytidylyltransferase activity like e.g., the NeuA enzyme from C. jejuni with SEQ ID NO:03, the NeuA enzyme from Haemophilus influenzae with SEQ ID NO:04 and the NeuA enzyme from Pasteurella multocida with SEQ ID NO:05 and to express one or more copies of a beta-galactoside alpha-2,3-sialyltransferase like e.g., SEQ ID NO:17 (PmultST3) from P. multocida, SEQ ID NO:18 (NmeniST3) from N. meningitidis or SEQ ID NO:48 (PmultST2) from P. multocida subsp. multocida str. Pm70, a beta-galactoside alpha-2,6-sialyltransferase like e.g., SEQ ID NO:19 (PdST6) from Photobacterium damselae or SEQ ID NO:20 (P-JT-ISH-224-ST6) from Photobacterium sp. JT-ISH-224, and/or an alpha-2,8-sialyltransferase like e.g., from M. musculus with SEQ ID NO:21.

Constitutive transcriptional units of the N-acylneuraminate cytidylyltransferases and the sialyltransferases can be delivered to the mutant strain either via genomic knock-in or via expression plasmids. If the mutant strains producing sialic acid and CMP-sialic acid were intended to make sialylated lactose structures, the strains were additionally modified with genomic knock-outs of the E. coli LacZ, LacY and LacA genes and with a genomic knock-in of a constitutive transcriptional unit for a lactose permease like e.g., E. coli LacY with SEQ ID NO:22. All mutant strains producing sialic acid, CMP-sialic acid and/or sialylated oligosaccharides could optionally be adapted for growth on sucrose via genomic knock-ins of constitutive transcriptional units containing a sucrose transporter like e.g., CscB from E. coli W with SEQ ID NO:23, a fructose kinase like e.g., Frk originating from Z. mobilis with SEQ ID NO:24 and a sucrose phosphorylase like e.g., from B. adolescentis with SEQ ID NO:25.

Alternatively, and/or additionally, sialic acid and/or sialylated oligosaccharide production can further be optimized in the mutant E. coli strains with a genomic knock-in of a constitutive transcriptional unit comprising a membrane transporter protein like e.g., a sialic acid transporter like e.g., nanT from E. coli K-12 MG1655 (UniProt ID P41036), nanT from E. coli 06:H1 (UniProt ID Q8FD59), nanT from E. coli O157:H7 (UniProt ID Q8X9G8) or nanT from E. albertii (UniProt ID B1EFH1) or a porter like e.g., EntS from E. coli (UniProt ID P24077), EntS from Kluyvera ascorbata (UniProt ID A0A378GQ13) or EntS from Salmonella enterica subsp. arizonae (UniProt ID A0A6Y2K4E8), MdfA from Cronobacter muytjensii (UniProt ID A0A2T7ANQ9), MdfA from Citrobacter youngae (UniProt ID D4BC23), MdfA from E. coli (UniProt ID POAEY8), MdfA from Yokenella regensburgei (UniProt ID G9Z5F4), iceT from E. coli (UniProt ID A0A024L207), iceT from Citrobacter youngae (UniProt ID D4B8A6), SetA from E. coli (UniProt ID P31675), SetB from E. coli (UniProt ID P33026) or SetC from E. coli (UniProt ID P31436) or an ABC transporter like e.g., oppF from E. coli (UniProt ID P77737), lmrA from Lactococcus lactis subsp. lactis bv. diacetylactis (UniProt ID A0A1VONEL4), or Blon_2475 from Bifidobacterium longum subsp. infantis (UniProt ID B7GPD4).

In an example for GDP-fucose production in the E. coli strains producing sialic acid, the mutant strains in these examples were further modified comprising knock-outs of the E. coli wcaJ and thyA genes and genomic knock-ins of constitutive transcriptional units containing a sucrose transporter like e.g., CscB from E. coli W with SEQ ID NO:23, a fructose kinase like e.g., Frk originating from Z. mobilis with SEQ ID NO:24 and a sucrose phosphorylase (SP) like e.g., from B. adolescentis with SEQ ID NO:25. For production of fucosylated oligosaccharides, the mutant GDP-fucose production strain was additionally modified with expression plasmids comprising constitutive transcriptional units for an alpha-1,2-fucosyltransferase like e.g., HpFutC from H. pylori with SEQ ID NO:26 and/or an alpha-1,3-fucosyltransferase like e.g., HpFucT from H. pylori with SEQ ID NO:27 and with a constitutive transcriptional unit for the E. coli thyA with SEQ ID NO:28 as selective marker. The constitutive transcriptional units of the fucosyltransferase genes could also be present in the mutant E. coli strain via genomic knock-ins. GDP-fucose production can further be optimized in the mutant E. coli strain by genomic knock-outs of any one or more of the E. coli genes comprising glgC, agp, pfkA, pfkB, pgi, arcA, icR, pgi and Ion as described in WO 2016075243 and WO 2012007481. GDP-fucose production can additionally be optimized comprising genomic knock-ins of constitutive transcriptional units for a mannose-6-phosphate isomerase like e.g., manA from E. coli with SEQ ID NO:29, a phosphomannomutase like e.g., manB from E. coli with SEQ ID NO:30, a mannose-1-phosphate guanylyltransferase like e.g., manC from E. coli with SEQ ID NO:31, a GDP-mannose 4,6-dehydratase like e.g., gmd from E. coli with SEQ ID NO:32 and a GDP-L-fucose synthase like e.g., fcl from E. coli with SEQ ID NO:33. GDP-fucose production can also be obtained by genomic knock-outs of the E. coli fucK and fucl genes and genomic knock-ins of constitutive transcriptional units containing a fucose permease like e.g., fucP from E. coli with SEQ ID NO:34 and a bifunctional fucose kinase/fucose-1-phosphate guanylyltransferase like e.g., fkp from Bacteroides fragilis with SEQ ID NO:35. If the mutant strains producing sialic acid and GDP-fucose were intended to make fucosylated lactose structures, the strains were additionally modified with genomic knock-outs of the E. coli LacZ, LacY and LacA genes and with a genomic knock-in of a constitutive transcriptional unit for a lactose permease like e.g., the E. coli LacY with SEQ ID NO:22. Furthermore, if the mutant strains were also intended to make sialylated structures, the strains were additionally modified with genomic knock-ins or expression plasmids comprising constitutive transcriptional units for one or more copies of an N-acylneuraminate cytidylyltransferase like e.g., NeuA from C. jejuni with SEQ ID NO:03, NeuA from H. influenzae with SEQ ID NO:04 and/or NeuA from P. multocida with SEQ ID NO:05 and to express one or more copies of a beta-galactoside alpha-2,3-sialyltransferase like e.g., SEQ ID NO:17 (PmultST3) from P. multocida, SEQ ID NO:18 (NmeniST3) from N. meningitidis or SEQ ID NO:48 (PmultST2) from P. multocida subsp. multocida str. Pm70, a beta-galactoside alpha-2,6-sialyltransferase like e.g., SEQ ID NO:19 (PdST6) from P. damselae and/or SEQ ID NO:20 (P-JT-ISH-224-ST6) from Photobacterium sp. JT-ISH-224, and/or an alpha-2,8-sialyltransferase like e.g., from M. musculus with SEQ ID NO:21.

Alternatively, and/or additionally, production of GDP-fucose and/or fucosylated structures can further be optimized in the mutant E. coli strains with genomic knock-ins of a constitutive transcriptional unit comprising a membrane transporter protein like e.g., MdfA from Cronobacter muytjensii (UniProt ID AOA2T7ANQ9), MdfA from Citrobacter youngae (UniProt ID D4BC23), MdfA from E. coli (UniProt ID POAEY8), MdfA from Yokenella regensburgei (UniProt ID G9Z5F4), iceT from E. coli (UniProt ID A0A024L207) or iceT from Citrobacter youngae (UniProt ID D4B8A6).

In an example for production of LN3 (GlcNAc-b1,3-Gal-b1,4-Glc) in the E. coli strains producing sialic acid, the mutant strains in these examples were further modified comprising genomic knock-outs of the E. coli LacZ, LacY and LacA genes and genomic knock-ins of constitutive transcriptional units for a lactose permease like e.g., the E. coli LacY with SEQ ID NO:22 and a galactoside beta-1,3-N-acetylglucosaminyltransferase like e.g., LgtA from N. meningitidis with SEQ ID NO:36.

In an example for production of LN-3 derived oligosaccharides like lacto-N-tetraose (LNT) and lacto-N-neotetraose (LNnT) the mutant LN3 producing strains were further modified with a constitutive transcriptional unit delivered to the strain either via genomic knock-in or from an expression plasmid for an N-acetylglucosamine beta-1,3-galactosyltransferase like e.g., WbgO from E. coli 055:H₇ with SEQ ID NO:37 to produce LNT or for an N-acetylglucosamine beta-1,4-galactosyltransferase like e.g., LgtB from N. meningitidis with SEQ ID NO:38 to produce LNnT. Optionally, multiple copies of the galactoside beta-1,3-N-acetylglucosaminyltransferase, the N-acetylglucosamine beta-1,3-galactosyltransferase and/or the N-acetylglucosamine beta-1,4-galactosyltransferase encoding genes could be added to the mutant E. coli strains. In addition, the strains can optionally be modified for enhanced UDP-galactose production with genomic knock-outs of the E. coli ushA and galT genes. The mutant E. coli strains can also optionally be adapted with a genomic knock-in of a constitutive transcriptional unit for an UDP-glucose-4-epimerase like e.g., galE from E. coli with SEQ ID NO:39. Furthermore, if the mutant strains were also intended to make sialylated structures, the strains were additionally modified with genomic knock-ins or expression plasmids comprising constitutive transcriptional units for one or more copies of an N-acylneuraminate cytidylyltransferase like e.g., NeuA from C. jejuni with SEQ ID NO:03, NeuA from H. influenzae with SEQ ID NO:04 and/or NeuA from P. multocida with SEQ ID NO:05 and to express one or more copies of a beta-galactoside alpha-2,3-sialyltransferase like e.g., SEQ ID NO:17 (PmultST3) from P. multocida, SEQ ID NO:18 (NmeniST3) from N. meningitidis or SEQ ID NO:48 (PmultST2) from P. multocida subsp. multocida str. Pm70, a beta-galactoside alpha-2,6-sialyltransferase like e.g., SEQ ID NO:19 (PdST6) from P. damselae and/or SEQ ID NO:20 (P-JT-ISH-224-ST6) from Photobacterium sp. JT-ISH-224, and/or an alpha-2,8-sialyltransferase like e.g., from M. musculus with SEQ ID NO:21. The mutant E. coli strains can also optionally be adapted for growth on sucrose via genomic knock-ins of constitutive transcriptional units containing a sucrose transporter like e.g., CscB from E. coli W with SEQ ID NO:23, a fructose kinase like e.g., Frk originating from Z. mobilis with SEQ ID NO:24 and a sucrose phosphorylase like e.g., from B. adolescentis with SEQ ID NO:25.

Alternatively, and/or additionally, production of LN3, LNT, LNnT and oligosaccharides derived thereof can further be optimized in the mutant E. coli strains with a genomic knock-in of a constitutive transcriptional unit comprising a membrane transporter protein like e.g., MdfA from Cronobacter muytjensii (UniProt ID A0A2T7ANQ9), MdfA from Citrobacter youngae (UniProt ID D4BC23), MdfA from E. coli (UniProt ID P0AEY8), MdfA from Yokenella regensburgei (UniProt ID G9Z5F4), iceT from E. coli (UniProt ID A0A024L207) or iceT from Citrobacter youngae (UniProt ID D4B8A6).Preferably but not necessarily, the glycosyltransferases, the proteins involved in nucleotide-activated sugar synthesis and/or membrane transporter proteins were N- and/or C-terminally fused to a solubility enhancer tag like e.g., a SUMO-tag, an MBP-tag, His, FLAG®, Strep-II, Halo-tag, NusA, thioredoxin, GST and/or the Fh8-tag to enhance their solubility (Costa et al., Front. Microbiol. 2014, doi.org/10.3389/fmicb.2014.00063; Fox et al., Protein Sci. 2001, 10(3), 622-630; Jia and Jeaon, Open Biol. 2016, 6: 160196).

Optionally, the mutant E. coli strains were modified with a genomic knock-in of a constitutive transcriptional unit encoding a chaperone protein like e.g., DnaK, DnaJ, GrpE, or the GroEL/ES chaperonin system (Baneyx F., Palumbo J. L. (2003) Improving Heterologous Protein Folding via Molecular Chaperone and Foldase Co-Expression. In: Vaillancourt P. E. (eds) E. coli Gene Expression Protocols. Methods in Molecular Biology™, vol 205. Humana Press).

Optionally, the mutant E. coli strains are modified to create a glycominimized E. coli strain comprising genomic knock-out of any one or more of non-essential glycosyltransferase genes comprising pgaC, pgaD, rfe, rffT, rffM, bcsA, bcsB, bcsC, wcaA, wcaC, wcaE, wcaI, wcaJ, wcaL, waaH, waaF, waaC, waaU, waaZ, waaJ, waaO, waafl, waaS, waaG, waaQ, wbbl, armC, arnT, yfdH, wbbK, opgG, opgH, ycjM, glgA, glgB, malQ, otsA and yaiP.

All constitutive promoters, UTRs and terminator sequences originated from the libraries described by Cambray et al. (Nucleic Acids Res. 2013, 41(9), 5139-5148), Dunn et al. (Nucleic Acids Res. 1980, 8, 2119-2132), Edens et al. (Nucleic Acids Res. 1975, 2, 1811-1820), Kim and Lee (FEBS Letters 1997, 407, 353-356) and Mutalik et al. (Nat. Methods 2013, No. 10, 354-360).

All genes were ordered synthetically at Twist Bioscience (twistbioscience.com) or IDT (eu.idtdna.com) and the codon usage was adapted using the tools of the supplier.

All strains were stored in cryovials at −80° C. (overnight LB culture mixed in a 1:1 ratio with 7000 glycerol).

TABLE 1
Overview of SEQ ID NOs described in the disclosure

| | | | | Country of |
|---|---|---|---|---|
| SEQ | | | | origin of digital |
| ID | | | | sequence |
| NO: | Name/identifier | Organism | Origin | information |
| 01 | NeuB | Neisseria meningitidis | Synthetic | United Kingdom |
| 02 | NeuB | Campylobacter jejuni | Synthetic | USA |
| 03 | NeuA | Campylobacter jejuni | Synthetic | USA |
| 04 | NeuA | Haemophilus influenzae | Synthetic | USA |
| 05 | NeuA | Pasteurella multocida | Synthetic | USA |
| 06 | glmS*54 | Escherichia coli K-12 | Synthetic | USA |
| | | MG1655 | | |
| 07 | GNA1 | Saccharomyces cerevisiae | Synthetic | USA |
| 08 | nanT (P41036) | Escherichia coli K-12 | Synthetic | USA |
| | | MG1655 | | |
| 09 | AGE | Bacteroides ovatus | Synthetic | USA |
| 10 | glmM | Escherichia coli K-12 | Synthetic | USA |
| | | MG1655 | | |
| 11 | glmU | Escherichia coli K-12 | Synthetic | USA |
| | | MG1655 | | |
| 12 | NeuC | Campylobacter jejuni | Synthetic | USA |
| 13 | GNE | Mus musculus (strain | Synthetic | USA |
| | | C57BL/6J) | | |
| 14 | NANS | Syntrophorhabdus sp. | Synthetic | USA |
| | | PtaU1.Bin058 | | |
| 15 | NANP | Candidatus Magnetomorum | Synthetic | Germany |
| | | sp. HK-1 | | |
| 16 | NANP | Bacteroides thetaiotaomicron | Synthetic | Unknown |
| 17 | alpha-2,3- | Pasteurella multocida | Synthetic | USA |
| | sialyltransferase | | | |
| 18 | alpha-2,3- | Neisseria meningitidis | Synthetic | United Kingdom |
| | sialyltransferase | | | |
| 19 | alpha-2,6- | Photobacterium damselae | Synthetic | Japan |
| | sialyltransferase | | | |
| 20 | alpha-2,6- | Photobacterium sp. JT-ISH- | Synthetic | Japan |
| | sialyltransferase | 224 | | |
| 21 | alpha-2,8- | Mus musculus | Synthetic | USA |
| | sialyltransferase | | | |
| 22 | LacY | Escherichia coli K-12 | Synthetic | USA |
| | | MG1655 | | |
| 23 | CscB | Escherichia coli W | Synthetic | USA |
| 24 | Frk | Zymomonas mobilis | Synthetic | United Kingdom |
| 25 | BaSP | Bifidobacterium adolescentis | Synthetic | Germany |
| 26 | HpFutC | Helicobacter pylori UA1234 | Synthetic | United Kingdom |
| 27 | HpFucT | Helicobacter pylori UA1234 | Synthetic | United Kingdom |
| 28 | thyA | Escherichia coli K-12 | Synthetic | USA |
| | | MG1655 | | |
| 29 | manA | Escherichia coli K-12 | Synthetic | USA |
| | | MG1655 | | |
| 30 | manB | Escherichia coli K-12 | Synthetic | USA |
| | | MG1655 | | |
| 31 | manC | Escherichia coli K-12 | Synthetic | USA |
| | | MG1655 | | |
| 32 | gmd | Escherichia coli K-12 | Synthetic | USA |
| | | MG1655 | | |
| 33 | fc1 | Escherichia coli K-12 | Synthetic | USA |
| | | MG1655 | | |
| 34 | fucP | Escherichia coli K-12 | Synthetic | USA |
| | | MG1655 | | |
| 35 | fkp | Bacteroides fragilis NCTC | Synthetic | United Kingdom |
| | | 9343 | | |
| 36 | lgtA | Neisseria meningitidis | Synthetic | United Kingdom |
| 37 | wbgO | Escherichia coli O55:H7 | Synthetic | Germany |
| 38 | lgtB | Neisseria meningitidis MC58 | Synthetic | United Kingdom |
| 39 | galE | Escherichia coli K-12 | Synthetic | USA |
| | | MG1655 | | |
| 40 | LAC12 | Kluyveromyces lactis | Synthetic | USA |
| 41 | ppsA | Escherichia coli K-12 | Synthetic | USA |
| | | MG1655 | | |
| 42 | PCK | Corynebacterium glutamicum | Synthetic | Denmark |
| 43 | pcka | Escherichia coli K-12 | Synthetic | USA |
| | | MG1655 | | |
| 44 | eda | Escherichia coli K-12 | Synthetic | USA |
| | | MG1655 | | |
| 45 | maeA | Escherichia coli K-12 | Synthetic | USA |
| | | MG1655 | | |
| 46 | maeB | Escherichia coli K-12 | Synthetic | USA |
| | | MG1655 | | |
| 47 | acs | Escherichia coli K-12 | Synthetic | USA |
| | | MG1655 | | |
| 48 | alpha-2,3- | Pasteurella multocida subsp. | Synthetic | Unknown |
| | sialyltransferase | multocida str. Pm70 | | |
| 49 | entS | Escherichia coli K-12 | Synthetic | USA |
| | | MG1655 | | |
| 50 | MdfA | Escherichia coli K-12 | Synthetic | USA |
| | | MG1655 | | |
| 51 | iceT | Escherichia coli K-12 | Synthetic | USA |
| | | MG1655 | | |
| 52 | oppF | Escherichia coli K-12 | Synthetic | USA |
| | | MG1655 | | |
| 53 | LmrA | Lactococcus lactis subsp. | Synthetic | Serbia |
| | | lactis bv. diacetylactis | | |
| 54 | Blon_2475 | Bifidobacterium longum | Synthetic | Germany |
| | | subsp. infantis | | |
| 55 | SetA | Escherichia coli K-12 | Synthetic | USA |
| | | MG1655 | | |
| 56 | SetB | Escherichia coli K-12 | Synthetic | USA |
| | | MG1655 | | |
| 57 | SetC | Escherichia coli K-12 | Synthetic | USA |
| | | MG1655 | | |

Cultivation Conditions

A preculture of 96-well microtiter plate experiments was started from a cryovial, in 150 μL LB and was incubated overnight at 37° C. on an orbital shaker at 800 rpm. This culture was used as inoculum for a 96-well square microtiter plate, with 400 μL minimal medium by diluting 400x. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor (having a 5 L working volume) was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2S04 and 20% NH4OH. The exhaust gas was cooled. 10% solution of silicone antifoaming agent was added when foaming raised during the fermentation.

Optical Density

Cell density of the cultures was frequently monitored by measuring optical density at 600 nm (Implen Nanophotometer NP80, Westburg, Belgium or with a Spark 10M microplate reader, Tecan, Switzerland).

Analytical Analysis

Standards such as but not limited to sucrose, lactose, N-acetyllactosamine (LacNAc, Gal-b1,4-GlcNAc), lacto-N-biose (LNB, Gal-b1,3-GlcNAc), fucosylated LacNAc (2′FLacNAc, 3-FLacNAc), sialylated LacNAc, (3′SLacNAc, 6′SLacNAc), fucosylated LNB (2′FLNB, 4′FLNB), lacto-N-triose II (LN3), lacto-N-tetraose (LNT), lacto-N-neo-tetraose (LNnT), LNFP-I, LNFP-II, LNFP-III, LNFP-V, LNFP-VI, LSTa, LSTc and LSTd were purchased from Carbosynth (UK), Elicityl (France) and IsoSep (Sweden). Other compounds were analyzed with in-house made standards.

Sialylated oligosaccharides were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C. Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a ¼ water and ¾ acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C. Both neutral and sialylated sugars were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analyzed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

### Example 3. Materials and Methods Saccharomyces cerevisiae

Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

Plasmids

In an example to produce sialic acid and CMP-sialic acid, a yeast expression plasmid can be derived from the pRS420-plasmid series (Christianson et al., 1992, Gene 110: 119-122) containing the TRP1 selection marker and constitutive transcriptional units for one or more copies of an L-glutamine-D-fructose-6-phosphate aminotransferase like e.g., the mutant glmS*54 from E. coli with SEQ ID NO:06, a phosphatase like any one or more of e.g., the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae and BsAraL from Bacillus subtilis as described in WO 2018122225, an N-acetylglucosamine 2-epimerase like e.g., AGE from B. ovatus with SEQ ID NO:09, one or more copies of an N-acetylneuraminate synthase like e.g., NeuB from N. meningitidis with SEQ ID NO:01 or from C. jejuni with SEQ ID NO:02, and one or more copies of an N-acylneuraminate cytidylyltransferase like e.g., NeuA from C. jejuni with SEQ ID NO:03, NeuA from H. influenzae with SEQ ID NO:04 and/or NeuA from P. multocida with SEQ ID NO:05. Optionally, a constitutive transcriptional unit comprising one or more copies for a glucosamine 6-phosphate N-acetyltransferase like e.g., GNA1 from S. cerevisiae with SEQ ID NO:07 was added as well.

To produce sialylated oligosaccharides, the plasmid further comprised constitutive transcriptional units for a lactose permease like e.g., LAC12 from Kluyveromyces lactis with SEQ ID NO:40, and one or more copies of a beta-galactoside alpha-2,3-sialyltransferase like e.g., SEQ ID NO:17 (PmultST3) from P. multocida, SEQ ID NO:18 (NmeniST3) from N. meningitidis or SEQ ID NO:48 (PmultST2) from P. multocida subsp. multocida str. Pm70, a beta-galactoside alpha-2,6-sialyltransferase like e.g., SEQ ID NO:19 (PdST6) from P. damselae and/or SEQ ID NO:20 (P-JT-ISH-224-ST6) from Photobacterium sp. JT-ISH-224, and/or an alpha-2,8-sialyltransferase like e.g., from M. musculus with SEQ ID NO:21.

In an example to produce GDP-fucose, a yeast expression plasmid like p2a_2μ_Fuc (Chan 2013, Plasmid 70, 2-17) can be used for expression of foreign genes in S. cerevisiae. This plasmid contains an ampicillin resistance gene and a bacterial origin of replication to allow for selection and maintenance in E. coli and the 2p yeast on and the Ura3 selection marker for selection and maintenance in yeast. This plasmid is further modified with constitutive transcriptional units for a lactose permease like e.g., LAC12 from K. lactis with SEQ ID NO:40, a GDP-mannose 4,6-dehydratase like e.g., gmd from E. coli with SEQ ID NO:32 and a GDP-L-fucose synthase like e.g., fcl from E. coli with SEQ ID NO:33. The yeast expression plasmid p2a_2μ_Fuc2 can be used as an alternative expression plasmid of the p2a_2μ_Fuc plasmid comprising next to the ampicillin resistance gene, the bacterial ori, the 2μ yeast on and the Ura3 selection marker constitutive transcriptional units for a lactose permease like e.g., LAC12 from K. lactis with SEQ ID NO:40, a fucose permease like e.g., fucP from E. coli with SEQ ID NO:34 and a bifunctional fucose kinase/fucose-1-phosphate guanylyltransferase like e.g., fkp from B. fragilis with SEQ ID NO:35. To further produce fucosylated oligosaccharides, the p2a_2μ_Fuc and its variant the p2a_2μ_Fuc2, additionally contained (a) constitutive transcriptional unit(s) for one or more fucosyltransferases like e.g., SEQ ID NOs:26 and 27.

In an example to produce UDP-galactose, a yeast expression plasmid can be derived from the pRS420-plasmid series (Christianson et al., 1992, Gene 110: 119-122) containing the HIS3 selection marker and a constitutive transcriptional unit for an UDP-glucose-4-epimerase like e.g., galE from E. coli with SEQ ID NO:39. This plasmid was further modified with constitutive transcriptional units for a lactose permease like e.g., LAC12 from K. lactis with SEQ ID NO:40, a galactoside beta-1,3-N-acetylglucosaminyltransferase like e.g., lgtA from N. meningitidis with SEQ ID NO:36 to produce LN3. To further produce LN3-derived oligosaccharides like LNT or LNnT, an N-acetylglucosamine beta-1,3-galactosyltransferase like e.g., WbgO from E. coli 055:H7 with SEQ ID NO:37 or an N-acetylglucosamine beta-1,4-galactosyltransferase like e.g., lgtB from N. meningitidis with SEQ ID NO:38, respectively, was also added on the plasmid.

Preferably but not necessarily, the glycosyltransferases were N-terminally fused to a SUMOstar tag (e.g., obtained from pYSUMOstar, Life Sensors, Malvern, PA) to enhance their solubility.

Plasmids were maintained in the host E. coli DH5alpha (F⁻, phi80dlacZdeltaM15, delta(lacZYA-argF)U169, deoR, recA1, endA1, hsdR17(rk⁻, mk⁺), phoA, supE44, lambda⁻, thi-1, gyrA96, relA1) bought from Invitrogen.

Heterologous and Homologous Expression

Genes that needed to be expressed, be it from a plasmid or from the genome were synthetically synthetized with one of the following companies: DNA2.0, Gen9, IDT or Twist Bioscience. Expression could be further facilitated by optimizing the codon usage to the codon usage of the expression host. Genes were optimized using the tools of the supplier.

Cultivations Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

Gene Expression Promoters

Genes were expressed using synthetic constitutive promoters, as described by Blazeck (Biotechnology and Bioengineering, Vol. 109, No. 11, 2012).

### Example 4. Production of 6′-sialyllactose (6′-SL) with a Modified E. coli Strain

An E. coli K-12 strain MG1655 was modified for sialic acid and 6′-siayllactose production as described in Example 2 comprising knock-outs of the E. coli nagA, nagB, nanA, nanT, nanE, nanK, LacZ, LacY and LacA genes and genomic knock-ins of constitutive transcriptional units containing the lactose permease (LacY) from E. coli with SEQ ID NO:22, the sialic acid transporter (nanT) from E. coli with SEQ ID NO:08, the L-glutamine D-fructose-6-phosphate aminotransferase (glmS*54) from E. coli with SEQ ID NO:06, the glucosamine 6-phosphate N-acetyltransferase (GNA1) from S. cerevisiae with SEQ ID NO:07, the N-acetylglucosamine 2-epimerase (AGE) from B. ovatus with SEQ ID NO:09, the N-acetylneuraminate synthase (NeuB) from C. jejuni with SEQ ID NO:02, the sucrose transporter (CscB) from E. coli W with SEQ ID NO:23, fructose kinase (Frk) from Z. mobilis with SEQ ID NO:24 and the sucrose phosphorylase from B. adolescentis with SEQ ID NO:25. The thus obtained mutant E. coli strain S0 was further modified with genomic knock-ins and/or expression plasmids with constitutive transcriptional units to express
a) one N-acylneuraminate cytidylyltransferase enzyme NeuA from C. jejuni with SEQ ID NO:03 and one beta-galactoside alpha-2,6-sialyltransferase PdbST from P. damselae with SEQ ID NO:19,
b) two N-acylneuraminate cytidylyltransferase isoproteins consisting of the NeuA enzyme from C. jejuni with SEQ ID NO:03 and the NeuA enzyme from H. influenzae with SEQ ID NO:04, and two copies of the beta-galactoside alpha-2,6-sialyltransferase PdbST from P. damselae with SEQ ID NO:19, or
c) three N-acylneuraminate cytidylyltransferase isoproteins consisting of the NeuA enzyme from C. jejuni with SEQ ID NO:03, the NeuA enzyme from H. influenzae with SEQ ID NO:04 and the NeuA enzyme from P. multocida with SEQ ID NO:05, and three copies of the beta-galactoside alpha-2,6-sialyltransferase PdbST from P. damselae with SEQ ID NO:19,
creating the mutant E. coli strains S1, S2 and S3, respectively, as summarized in Table 2. Details on the promoter, UTR and terminator sequences used to express the NeuA enzymes or PdbST is summarized in Table 3. The novel strains were evaluated in a growth experiment according to the culture conditions provided in Example 2, in which the culture medium contained 30 g/L sucrose and 20 g/L lactose. Each strain was grown in four biological replicates in a 96-well plate. After 72 h of incubation, the culture broth was harvested, and the sugars were analyzed on UPLC.

The experiment demonstrated all novel strains produced 6′-SL. Herein, strain S2 expressing two isoproteins with N-acylneuraminate cytidylyltransferase activity, i.e., SEQ ID NOs:03 and 04, and two copies of the PdbST enzyme from P. damselae with SEQ TD NO:19 produced 2.60 times more 6′-SL compared to strain S1 expressing one NeuA enzyme from C. jejuni with SEQ ID NO:03 and one copy of the PdbST enzyme from P. damselae with SEQ ID NO: 19. In the same experiment, strain S3 expressing three isoproteins with N-acylneuraminate cytidylyltransferase activity, i.e., SEQ TD NOs:03, 04 and 05, and three copies of the PdbST enzyme from P. damselae with SEQ TD NO:19 produced 11.50 times more 6′-SL compared to strain 1 expressing one NeuA enzyme from C. jejuni with SEQ ID NO:03 and one copy of the PdbST enzyme from P. damselae with SEQ ID NO:19. The experiment further demonstrated all mutant strains had a similar growth rate and did not suffer from any genomic or plasmid DNA instability or reorganization during cultivation (Results not shown).

TABLE 2
Additional transcriptional units present in E. coli strain S1, S2 and S3
compared to the parental E. coli strain S0

| | | Transcriptional unit | | | |
|---|---|---|---|---|---|
| | | Pro- | | | Ter- |
| | | moter | UTR | | minator |
| | | se- | se- | | se- |
| Strain | Location | quence* | quence* | Coding DNA sequence | quence* |
| S1 | Genomic | P35 | U18 | CjNeuA = SEQ ID NO: 3 | T2 |
| | knock-in | | | | |
| | Genomic | P50 | U10 | PdbST = SEQ ID NO: 19 | T1 |
| | knock-in | | | | |
| S2 | Genomic | P35 | U18 | CjNeuA = SEQ ID NO: 3 | T2 |
| | knock-in | | | | |
| | Genomic | P26 | U18 | HiNeuA = SEQ ID | T9 |
| | knock-in | | | NO: 04 | |
| | Genomic | P50 | U10 | PdbST = SEQ ID NO: 19 | T1 |
| | knock-in | | | | |
| | Genomic | P5 | U10 | PdbST = SEQ ID NO: 19 | T7 |
| | knock-in | | | | |
| S3 | Genomic | P35 | U18 | CjNeuA = SEQ ID NO: 3 | T2 |
| | knock-in | | | | |
| | Genomic | P26 | U18 | HiNeuA = SEQ ID | T9 |
| | knock-in | | | NO: 04 | |
| | Plasmid | P26 | U18 | PmultNeuA = SEQ ID | T9 |
| | | | | NO: 05 | |
| | Genomic | P50 | U10 | PdbST = SEQ ID NO: 19 | T1 |
| | knock-in | | | | |
| | Genomic | P5 | U10 | PdbST = SEQ ID NO: 19 | T7 |
| | knock-in | | | | |
| | Plasmid | P5 | U10 | PdbST = SEQ ID NO: 19 | T7 |

*See Table 3

TABLE 3
Promoter, UTR and terminator sequences used to express the neuA
isoproteins or the alpha-2,6-sialyltransferase PdbST in the mutant E. coli strains S1, S2 and S3 as
given in Table 2

| Promoter sequence | Reference |
|---|---|
| P5 = PROM0005 = Mutalik_P5 | Mutalik et al. (Nat. Methods 2013, 10, 354-360) |
| P26 = PROM0026 = | Mutalik et al. (Nat. Methods 2013, 10, 354-360) |
| Mutalik_apFAB110 | |
| P35 = PROM0035 = Mutalik_apFAB37 | Mutalik et al. (Nat. Methods 2013, 10, 354-360) |
| P50 = PROM0050 = Mutalik_apFAB82 | Mutalik et al. (Nat. Methods 2013, 10, 354-360) |
| UTR sequence | Reference |
| U10= UTR0010_GalE_BCD12 | Mutalik et al. (Nat. Methods 2013, 10, 354-360) |
| U18= UTR0018_GalE_BCD18 | Mutalik et al. (Nat. Methods 2013, 10, 354-360) |
| Terminator sequence | Reference |
| T1 = TER0001_TT5-T7 | Dunn et al. (Nucleic Acids Res. 1980, 8, 2119-2132) |
| T2 = TER0002_rrnBT1_rrnBT2 | Kim and Lee (FEBS Letters 1997, 407, 353-356) |
| T7 = TER0007_ilvGEDA | Cambray et al. (Nucleic Acids Res. 2013, 41, 5139- |
| | 5148) |
| T9 = TER0009_M13_central | Edens et al. (Nucleic Acids Res. 1975, 2, 1811-1820) |

### Example 5. Production of 3′-Sialyllactose (3′-SL) with a Modified E. coli Strain

An E. coli K-12 strain MG1655 was modified for sialic acid and 3′-siayllactose production as described in Example 2 comprising knock-outs of the E. coli nagA, nagB, nanA, nanT, nanE, nanK, LacZ, LacY and LacA genes and genomic knock-ins of constitutive transcriptional units containing the lactose permease (LacY) from E. coli with SEQ ID NO:22, the sialic acid transporter (nanT) from E. coli with SEQ ID NO: 08, two copies of the L-glutamine D-fructose-6-phosphate aminotransferase (glmS*54) from E. coli with SEQ ID NO:06, two copies of the glucosamine 6-phosphate N-acetyltransferase (GNA1) from S. cerevisiae with SEQ ID NO:07, the N-acetylglucosamine 2-epimerase (AGE) from B. ovatus with SEQ ID NO:09, the N-acetylneuraminate synthase (NeuB) from N. meningitidis with SEQ ID NO:01, the sucrose transporter (CscB) from E. coli W with SEQ ID NO:23, fructose kinase (Frk) from Z. mobilis with SEQ ID NO:24 and the sucrose phosphorylase from B. adolescentis with SEQ ID NO:25. The thus obtained mutant E. coli strain S30 was further modified with genomic knock-ins of constitutive transcriptional units to express
a) one copy of the N-acylneuraminate cytidylyltransferase enzyme from P. multocida with SEQ ID NO:05 and one copy of the beta-galactoside alpha-2,3-sialyltransferase PmultST3 from P. multocida with SEQ ID NO:17, or
b) two copies of the NeuA enzyme from P. multocida with SEQ ID NO:05 and two copies of the PmultST3 sialyltransferase from P. multocida with SEQ ID NO:17,
creating the mutant E. coli strains S4 and S5, respectively, as summarized in Table 4. Details on the promoter, UTR and terminator sequences used to express the NeuA enzymes or PmultST3 is summarized in Table 5. The novel strains were evaluated in a growth experiment according to the culture conditions provided in Example 2, in which the culture medium contained 30 g/L sucrose and 20 g/L lactose. Each strain was grown in four biological replicates in a 96-well plate. After 72 h of incubation, the culture broth was harvested, and the sugars were analyzed on UPLC.

The experiment demonstrated both novel strains S4 and S5 produced 3′-SL. Herein, strain S4 expressing two copies of the NeuA enzyme from P. multocida with SEQ ID NO:05, and two copies of the PmultST3 enzyme from P. multocida with SEQ ID NO:17 produced 3.70 times more 3′-SL compared to strain S30 expressing one copy of the NeuA enzyme from P. multocida with SEQ ID NO:05, and one copy of the PmultST3 enzyme from P. multocida with SEQ ID NO:17. The experiment further demonstrated that both mutant strains S4 and S5 had a similar growth rate and did not suffer from any genomic or plasmid DNA instability or reorganization during cultivation.

TABLE 4
Additional transcriptional units present in E. coli strain S4 and S5
compared to the parental E. coli strain S30

| | | Transcriptional unit | | | |
|---|---|---|---|---|---|
| | | Promoter | UTR | | Ter- |
| | | se- | se- | | minator |
| Strain | Location | quence* | quence* | Coding DNA sequence | sequence* |
| S4 | Genomic | P26 | U18 | PmultNeuA = SEQ ID | T9 |
| | knock-in | | | NO: 05 | |
| | Genomic | P50 | U14 | PmultST3 = SEQ ID | T7 |
| | knock-in | | | NO: 17 | |
| S5 | Genomic | P26 | U18 | PmultNeuA = SEQ ID | T9 |
| | knock-in | | | NO: 05 | |
| | Genomic | P26 | U11 | PmultNeuA = SEQ ID | T9 |
| | knock-in | | | NO: 05 | |
| | Genomic | P50 | U14 | PmultST3 = SEQ ID | T7 |
| | knock-in | | | NO: 17 | |
| | Genomic | P50 | U14 | PmultST3 = SEQ ID | T7 |
| | knock-in | | | NO: 17 | |

*See Table 5

TABLE 5
Promoter, UTR and terminator sequences used to express PmultNeuA
or alpha-2,3-sialyltransferase PmultST3 in the mutant E. coli strains S4 and S5 as given in Table 4

| Promoter sequence | Reference |
|---|---|
| P26 = PROM0026 = | Mutalik et al. (Nat. Methods 2013, 10, 354-360) |
| Mutalik_apFAB110 | |
| P50 = PROM0050 = Mutalik_apFAB82 | Mutalik et al. (Nat. Methods 2013, 10, 354-360) |
| UTR sequence | Reference |
| UTR11 = UTR0011_Gene10_LeuL | Mutalik et al. (Nat. Methods 2013, 10, 354-360) |
| U14 = UTR0014_GalE_LeuAB | Mutalik et al. (Nat. Methods 2013, 10, 354-360) |
| U18 = UTR0018_GalE_BCD18 | Mutalik et al. (Nat. Methods 2013, 10, 354-360) |
| Terminator sequence | Reference |
| T7 = TER0007_ilvGEDA | Cambray et al. (Nucleic Acids Res. 2013, 41, 5139- |
| | 5148) |
| T9 = TER0009_M13_central | Edens et al. (Nucleic Acids Res. 1975, 2, 1811-1820) |

### Example 6. Production of 6′-Sialyllactose (6′-SL) with a Modified E. coli Strain

In a first step, an E. coli K-12 strain MG1655 was modified comprising knock-outs of the E. coli nagA, nagB, nanA, nanT, nanE, nanK, LacZ, LacY, LacA, ackA-pta, ldhA and poxB genes and the O-antigen cluster comprising all genes between wbbK and wcaN with wbbK and wcaN included. In a next step, the mutant strain was further modified with genomic knock-ins of constitutive transcriptional units containing the lactose permease (LacY) from E. coli with SEQ ID NO:22, the sialic acid transporter (nanT) from E. coli with SEQ ID NO:08, two copies of the L-glutamine D-fructose-6-phosphate aminotransferase (glmS*54) from E. coli with SEQ ID NO:06, two copies of the glucosamine 6-phosphate N-acetyltransferase (GNA1) from S. cerevisiae with SEQ ID NO:07, the N-acetylglucosamine 2-epimerase (AGE) from B. ovatus with SEQ ID NO:09, the N-acetylneuraminate synthase (NeuB) from N. meningitidis with SEQ ID NO:01, the acetyl-coenzyme A synthetase (acs) from E. coli with SEQ ID NO:47, the sucrose transporter (CscB) from E. coli W with SEQ ID NO:23, fructose kinase (Frk) from Z. mobilis with SEQ ID NO:24 and the sucrose phosphorylase from B. adolescentis with SEQ ID NO:25. The thus obtained mutant E. coli strain was further modified with genomic knock-ins and an expression plasmid with constitutive transcriptional units to express three N-acylneuraminate cytidylyltransferase isoproteins consisting of the NeuA enzyme from C. jejuni with SEQ ID NO:03, the NeuA enzyme from H. influenzae with SEQ ID NO:04 and the NeuA enzyme from P. multocida with SEQ ID NO:05, and three copies of the beta-galactoside alpha-2,6-sialyltransferase PdbST from P. damselae with SEQ ID NO:19. The novel strain was evaluated in a growth experiment according to the culture conditions provided in Example 2, in which the culture medium contained 30 g/L sucrose and 20 g/L lactose. The strain was grown in four biological replicates in a 96-well plate. After 72 h of incubation, the culture broth was harvested, and the sugars were analyzed on UPLC. The experiment demonstrated the novel strain produced sialic acid (Neu5Ac) and 6′-SL and did not suffer from any genomic or plasmid DNA instability or reorganization during cultivation.

### Example 7. Production of 3′-Sialyllactose (3′-SL) with a Modified E. coli Strain

An E. coli K-12 strain MG1655 is modified for sialic acid and 3′-siayllactose production as described in Example 2 comprising knock-outs of the E. coli nagA, nagB, nanA, nanT, nanE, nanK, LacZ, LacY and LacA genes and genomic knock-ins of constitutive transcriptional units containing the lactose permease (LacY) from E. coli with SEQ ID NO:22, the sialic acid transporter (nanT) from E. coli with SEQ ID NO:08, two copies of the L-glutamine D-fructose-6-phosphate aminotransferase (glmS*54) from E. coli with SEQ ID NO:06, two copies of the glucosamine 6-phosphate N-acetyltransferase (GNA1) from S. cerevisiae with SEQ ID NO:07, the N-acetylglucosamine 2-epimerase (AGE) from B. ovatus with SEQ ID NO:09, the N-acetylneuraminate synthase (NeuB) from N. meningitidis with SEQ ID NO:01, the sucrose transporter (CscB) from E. coli W with SEQ ID NO:23, fructose kinase (Frk) from Z. mobilis with SEQ ID NO:24 and the sucrose phosphorylase from B. adolescentis with SEQ ID NO:25. The thus obtained mutant E. coli strain is further modified with genomic knock-ins and an expression plasmid with constitutive transcriptional units to express three N-acylneuraminate cytidylyltransferase isoproteins consisting of the NeuA enzyme from C. jejuni with SEQ ID NO:03, the NeuA enzyme from H. influenzae with SEQ ID NO:04 and the NeuA enzyme from P. multocida with SEQ ID NO:05, and three copies of the beta-galactoside alpha-2,3-sialyltransferase PmultST3 from P. multocida with SEQ ID NO:17. The novel strain is evaluated in a growth experiment according to the culture conditions provided in Example 2, in which the culture medium contains 30 g/L sucrose and 20 g/L lactose. The strain is grown in four biological replicates in a 96-well plate. After 72 h of incubation, the culture broth is harvested, and the sugars are analyzed on UPLC.

### Example 8. Production of 3′-Sialyllactose (3′-SL) with a Modified E. coli Strain

An E. coli K-12 strain MG1655 was modified for sialic acid and 3′-siayllactose production as described in Example 2 comprising knock-outs of the E. coli nagA, nagB, nanA, nanT, nanE, nanK, LacZ, LacY and LacA genes and genomic knock-ins of constitutive transcriptional units containing the lactose permease (LacY) from E. coli with SEQ ID NO:22, the sialic acid transporter (nanT) from E. coli with SEQ ID NO:08, two copies of the L-glutamine D-fructose-6-phosphate aminotransferase (glmS*54) from E. coli with SEQ ID NO:06, two copies of the glucosamine 6-phosphate N-acetyltransferase (GNA1) from S. cerevisiae with SEQ ID NO:07, the N-acetylglucosamine 2-epimerase (AGE) from B. ovatus with SEQ ID NO:09, the N-acetylneuraminate synthase (NeuB) from N. meningitidis with SEQ ID NO:01, the sucrose transporter (CscB) from E. coli W with SEQ ID NO:23, fructose kinase (Frk) from Z. mobilis with SEQ ID NO:24 and the sucrose phosphorylase from B. adolescentis with SEQ ID NO:25. The thus obtained mutant E. coli strain was further modified with genomic knock-ins with constitutive transcriptional units to express two copies of the N-acylneuraminate cytidylyltransferase NeuA from P. multocida with SEQ ID NO:05 and two copies of the beta-galactoside alpha-2,3-sialyltransferase PmultST3 from P. multocida with SEQ ID NO:17. The novel strain was evaluated in a growth experiment according to the culture conditions provided in Example 2, in which the culture medium contained 30 g/L sucrose and 20 g/L lactose. The strain was grown in four biological replicates in a 96-well plate. After 72 h of incubation, the culture broth was harvested, and the sugars were analyzed on UPLC. The experiment demonstrated the novel strain produced 0.64 g/L 3′-SL and did not suffer from any genomic or plasmid DNA instability or reorganization during cultivation.

### Example 9. Evaluation of Mutant E. coli 6′-SL Production Strains in Fed-Batch Fermentations

The mutant E. coli strains as described in Examples 4 and 6 were evaluated in a fed-batch fermentation process. Fed-batch fermentations at bioreactor scale were performed as described in Example 2. Sucrose was used as a carbon source and lactose was added in the batch medium as a precursor. No sialic acid (Neu5Ac) was added to the fermentation process. In contrast to the cultivation experiments that are described herein and wherein only end samples were taken at the end of cultivation (i.e., 72 hours as described herein), regular broth samples were taken at several time points during the fermentation process and the production of sialic acid (Neu5Ac) and 6′-sialyllactose at each of the time points was measured using UPLC as described in Example 2. The experiment demonstrated that broth samples taken e.g., at the end of the batch phase and during fed-batch phase comprised sialic acid production together with 6′-sialyllactose and unmodified lactose. Broth samples taken at the end of the fed-batch phase comprised 6′-sialyllactose and almost no or a very low concentration of Neu5Ac and almost no or a very low concentration of unmodified lactose demonstrating almost all or all of the precursor lactose was modified with almost all or all Neu5Ac produced during the fermentation of the mutant cells producing 6′-SL. The experiment further showed the mutant strains did not suffer from any genomic or plasmid DNA instability or reorganization during cultivation.

### Example 10. Evaluation of Mutant E. coli 3′-SL Production Strains in Fed-Batch Fermentations

The mutant E. coli strains as described in Examples 5, 7 and 8 are evaluated in a fed-batch fermentation process. Fed-batch fermentations at bioreactor scale are performed as described in Example 2. Sucrose is used as a carbon source and lactose is added in the batch medium as a precursor. No sialic acid (Neu5Ac) is added to the fermentation process. In contrast to the cultivation experiments that are described herein and wherein only end samples were taken at the end of cultivation (i.e., 72 hours as described herein), regular broth samples are taken at several time points during the fermentation process and the production of sialic acid (Neu5Ac) and 3′-sialyllactose at each of the time points is measured using UPLC as described in Example 2.

### Example 11. Production of an Oligosaccharide Mixture Comprising 6′-SL, LN3, Sialylated LN3, LNnT and LSTc with a Modified E. coli Host

An E. coli host modified for sialic acid production (Neu5Ac) and 6′-siayllactose as described in Example 6 is further modified with genomic knock-ins comprising constitutive transcriptional units of the galactoside beta-1,3-N-acetylglucosaminyltransferase (LgtA) from N. meningitidis with SEQ ID NO:36 and the N-acetylglucosamine beta-1,4-galactosyltransferase (LgtB) from N. meningitidis with SEQ ID NO:38 to produce a mixture of oligosaccharides comprising 6′-SL, LN3, LNnT and LSTc (Neu5Ac-a2,6-Gal-b1,4-GlcNAc-b1,3-Gal-b1,4-Glc). The novel strain is evaluated in a growth experiment according to the culture conditions provided in Example 2, in which the culture medium contains sucrose and lactose. The strain is grown in four biological replicates in a 96-well plate. After 72 h of incubation, the culture broth is harvested, and the sugars are analyzed on UPLC.

### Example 12. Production of an Oligosaccharide Mixture LN3, Sialylated LN3, LNT, 3′-SL and LSTa with a Modified E. coli Host

An E. coli host modified for sialic acid production (Neu5Ac) and 3′-siayllactose as described in Example 8 is further modified with genomic knock-ins comprising constitutive transcriptional units of the galactoside beta-1,3-N-acetylglucosaminyltransferase (LgtA) from N. meningitidis with SEQ ID NO:36 and the N-acetylglucosamine beta-1,3-galactosyltransferase (wbgO) from E. coli 055:H7 with SEQ ID NO:37 to produce a mixture of oligosaccharides comprising LN3, 3′-sialylated LN3 (Neu5Ac-a2,3-GlcNAc-b1,3-Gal-b1,4-Glc), LNT, 3′-SL and LSTa (Neu5Ac-a2,3-Gal-b1,3-GlcNAc-b1,3-Gal-b1,4-Glc). The novel strain is evaluated in a growth experiment according to the culture conditions provided in Example 2, in which the culture medium contains sucrose and lactose. The strain is grown in four biological replicates in a 96-well plate. After 72 h of incubation, the culture broth is harvested, and the sugars are analyzed on UPLC.

### Example 13. Production of an Oligosaccharide Mixture Comprising LN3, Sialylated LN3, LNnT, 3′-SL and LSTd with a Modified E. coli Host

An E. coli host modified for sialic acid production (Neu5Ac) and 3′-siayllactose as described in Example 8 is further modified with genomic knock-ins comprising constitutive transcriptional units of the galactoside beta-1,3-N-acetylglucosaminyltransferase (LgtA) from N. meningitidis with SEQ ID NO:36 and the N-acetylglucosamine beta-1,4-galactosyltransferase (LgtB) from N. meningitidis with SEQ ID NO:38 to produce a mixture of oligosaccharides comprising 3′-SL, LN3, 3′-sialylated LN3 (Neu5Ac-a2,3-GlcNAc-b1,3-Gal-b1,4-Glc), LNnT and LSTd (Neu5Ac-a2,3-Gal-b1,4-GlcNAc-b1,3-Gal-b1,4-Glc). The novel strain is evaluated in a growth experiment according to the culture conditions provided in Example 2, in which the culture medium contains sucrose and lactose. The strain is grown in four biological replicates in a 96-well plate. After 72 h of incubation, the culture broth is harvested, and the sugars are analyzed on UPLC.

### Example 14. Production of 6′-Sialyllactose (6′-SL) with a Modified E. coli Strain

An E. coli K-12 strain MG1655 was modified for sialic acid and 6′-siayllactose production as described in Example 2 comprising knock-outs of the E. coli nagA, nagB, nanA, nanT, nanE, nanK, LacZ, LacY and LacA genes and genomic knock-ins of constitutive transcriptional units containing the lactose permease (LacY) from E. coli with SEQ ID NO:22, the sialic acid transporter (nanT) from E. coli with SEQ ID NO:08, two copies of the L-glutamine D-fructose-6-phosphate aminotransferase (glmS*54) from E. coli with SEQ ID NO:06, the phosphoglucosamine mutase (glmM) from E. coli with SEQ ID NO:10, the N-acetylglucosamine-1-phosphate uridyltransferase/glucosamine-1-phosphate acetyltransferase (glmU) from E. coli with SEQ ID NO:11, the UDP-N-acetylglucosamine 2-epimerase (NeuC) from C. jejuni with SEQ ID NO:12, the N-acetylneuraminate synthase (NeuB) from meningitides with SEQ ID NO:01, the sucrose transporter (CscB) from E. coli W with SEQ ID NO:23, fructose kinase (Frk) from Z mobilis with SEQ ID NO:24 and the sucrose phosphorylase from B. adolescentis with SEQ ID NO:25. The thus obtained mutant E. coli strain was further modified with genomic knock-ins and an expression plasmid with constitutive transcriptional units to express three N-acylneuraminate cytidylyltransferase isoproteins consisting of the NeuA enzyme from C. jejuni with SEQ ID NO:03, the NeuA enzyme from H. influenzae with SEQ ID NO:04 and the NeuA enzyme from P. multocida with SEQ ID NO:05, and three copies of the beta-galactoside alpha-2,6-sialyltransferase PdbST from P. damselae with SEQ ID NO: 19. The novel strain is evaluated in a growth experiment according to the culture conditions provided in Example 2, in which the culture medium contains 30 g/L sucrose and 20 g/L lactose. The strain is grown in four biological replicates in a 96-well plate. After 72 h of incubation, the culture broth is harvested, and the sugars are analyzed on UPLC.

### Example 15. Production of 6′-Sialyllactose (6′-SL) with a Modified E. coli Strain

An E. coli K-12 strain MG1655 was modified for sialic acid and 6′-siayllactose production as described in Example 2 comprising knock-outs of the E. coli nagA, nagB, nanA, nanT, nanE, nanK, LacZ, LacY and LacA genes and genomic knock-ins of constitutive transcriptional units containing the lactose permease (LacY) from E. coli with SEQ ID NO:22, the sialic acid transporter (nanT) from E. coli with SEQ ID NO:08, two copies of the L-glutamine D-fructose-6-phosphate aminotransferase (glmS*54) from E. coli with SEQ ID NO:06, the phosphoglucosamine mutase (glmM) from E. coli with SEQ ID NO:10, the N-acetylglucosamine-1-phosphate uridyltransferase/glucosamine-1-phosphate acetyltransferase (glmU) from E. coli with SEQ ID NO:11, the bifunctional UDP-GlcNAc 2-epimerase/N-acetylmannosamine kinase from M. musculus (strain C57BL/6J) with SEQ ID NO:13, the N-acylneuraminate-9-phosphate synthetase from Syntrophorhabdus sp. PtaU1.Bin058 with SEQ ID NO:14 and the N-acylneuraminate-9-phosphatase from Candidatus magnetomorum sp. HK-1 with SEQ ID NO:15, the sucrose transporter (CscB) from E. coli W with SEQ ID NO:23, fructose kinase (Frk) from Z. mobilis with SEQ ID NO:24 and the sucrose phosphorylase from B. adolescentis with SEQ ID NO:25. The thus obtained mutant E. coli strain was further modified with genomic knock-ins and an expression plasmid with constitutive transcriptional units to express three N-acylneuraminate cytidylyltransferase isoproteins consisting of the NeuA enzyme from C. jejuni with SEQ ID NO:03, the NeuA enzyme from H. influenzae with SEQ ID NO:04 and the NeuA enzyme from P. multocida with SEQ ID NO:05, and three copies of the beta-galactoside alpha-2,6-sialyltransferase PdbST from P. damselae with SEQ ID NO: 19. The novel strain is evaluated in a growth experiment according to the culture conditions provided in Example 2, in which the culture medium contains 30 g/L sucrose and 20 g/L lactose. The strain is grown in four biological replicates in a 96-well plate. After 72 h of incubation, the culture broth is harvested, and the sugars are analyzed on UPLC.

### Example 16. Production of 6′-Sialyllactose (6′-SL) with a Modified S. cerevisiae Strain

An S. cerevisiae strain is adapted for sialic acid (Neu5Ac) and sialylated lactose production as described in Example 3 with a pRS420-derived yeast expression plasmid comprising the TRP1 selection marker and constitutive transcriptional units for two copies of the L-glutamine D-fructose-6-phosphate aminotransferase (glmS*54) from E. coli with SEQ ID NO:06, a phosphatase like any one or more of e.g., the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from P. putida, ScDOG1 from S. cerevisiae and BsAraL from B. subtilis as described in WO 2018122225, the N-acetylglucosamine 2-epimerase (AGE) from B. ovatus with SEQ ID NO:09, the N-acetylneuraminate synthase (NeuB) from N. meningitidis with SEQ ID NO:01, three N-acylneuraminate cytidylyltransferase isoproteins consisting of the NeuA enzyme from C. jejuni with SEQ ID NO:03, the NeuA enzyme from H. influenzae with SEQ ID NO:04 and the NeuA enzyme from P. multocida with SEQ ID NO:05, three copies of the beta-galactoside alpha-2,6-sialyltransferase PdbST from P. damselae with SEQ ID NO:19 and the lactose permease (LAC12) from K. lactis with SEQ ID NO:40. The novel strain is evaluated in a growth experiment on SD CSM-Trp drop-out medium comprising lactose as precursor according to the culture conditions provided in Example 3. After 72 h of incubation, the culture broth is harvested, and the sugars are analyzed on UPLC.

### Example 17. Production of an Oligosaccharide Mixture Comprising 6′-SL, LN3, Sialylated LN3, LNnT and LSTc with a Modified S. cerevisiae Host

The mutant S. cerevisiae strain described in Example 16 is further modified with a second pRS420-derived yeast expression plasmid comprising the HIS3 selection marker and constitutive transcriptional units for galE from E. coli with SEQ ID NO:39, the galactoside beta-1,3-N-acetylglucosaminyltransferase (lgtA) from N. meningitidis with SEQ ID NO:36 and the N-acetylglucosamine beta-1,4-galactosyltransferase (lgtB) from N. meningitidis with SEQ ID NO:38 to produce a mixture of oligosaccharides comprising 6′-SL, LN3, LNnT and LSTc (Neu5Ac-a2,6-Gal-b1,4-GlcNAc-b1,3-Gal-b1,4-Glc). The novel strain is evaluated in a growth experiment on SD CSM-Trp-His drop-out medium comprising lactose as precursor according to the culture conditions provided in Example 3. After 72 h of incubation, the culture broth is harvested, and the sugars are analyzed on UPLC.

### Example 18. Production of 3′-Sialyllactose (3′-SL) with a Modified S. cerevisiae Strain

An S. cerevisiae strain is adapted for sialic acid (Neu5Ac) and sialylated lactose production as described in Example 3 with a pRS420-derived yeast expression plasmid comprising the TRP1 selection marker and constitutive transcriptional units for two copies of the L-glutamine D-fructose-6-phosphate aminotransferase (glmS*54) from E. coli with SEQ ID NO:06, a phosphatase like any one or more of e.g., the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from P. putida, ScDOG1 from S. cerevisiae and BsAraL from B. subtilis as described in WO 2018122225, the N-acetylglucosamine 2-epimerase (AGE) from B. ovatus with SEQ ID NO:09, the N-acetylneuraminate synthase (NeuB) from N. meningitidis with SEQ ID NO:01, three N-acylneuraminate cytidylyltransferase isoproteins consisting of the NeuA enzyme from C. jejuni with SEQ ID NO:03, the NeuA enzyme from H. influenzae with SEQ ID NO:04 and the NeuA enzyme from P. multocida with SEQ ID NO:05, three copies of the beta-galactoside alpha-2,3-sialyltransferase PmultST3 from P. multocida with SEQ ID NO:17 and the lactose permease (LAC12) from K. lactis with SEQ ID NO:40. The novel strain is evaluated in a growth experiment on SD CSM-Trp drop-out medium comprising lactose as precursor according to the culture conditions provided in Example 3. After 72 h of incubation, the culture broth is harvested, and the sugars are analyzed on UPLC.

### Example 19. Production of an Oligosaccharide Mixture Comprising LN3, Sialylated LN3, LNT, 3′-SL and LSTa with a Modified S. cerevisiae Host

The mutant S. cerevisiae strain described in Example 18 is further modified with a second pRS420-derived yeast expression plasmid comprising the HIS3 selection marker and constitutive transcriptional units for galE from E. coli with SEQ ID NO:39, the galactoside beta-1,3-N-acetylglucosaminyltransferase (lgtA) from N. meningitidis with SEQ ID NO:36 and the N-acetylglucosamine beta-1,3-galactosyltransferase (wbgO) from E. coli 055:H7 with SEQ ID NO:37 to produce a mixture of oligosaccharides comprising LN3, 3′-sialylated LN3 (Neu5Ac-a2,3-GlcNAc-b1,3-Gal-b1,4-Glc), LNT, 3′-SL and LSTa (Neu5Ac-a2,3-Gal-b1,3-GlcNAc-b1,3-Gal-b1,4-Glc). The novel strain is evaluated in a growth experiment on SD CSM-Trp-His drop-out medium comprising lactose as precursor according to the culture conditions provided in Example 3. After 72 h of incubation, the culture broth is harvested, and the sugars are analyzed on UPLC.

### Example 20. Production of an Oligosaccharide Mixture Comprising LN3, Sialylated LN3, LNnT, 3′-SL and LSTd with a Modified S. cerevisiae Host

The mutant S. cerevisiae strain described in Example 18 is further modified with a second pRS420-derived yeast expression plasmid comprising the HIS3 selection marker and constitutive transcriptional units for galE from E. coli with SEQ ID NO:39, the galactoside beta-1,3-N-acetylglucosaminyltransferase (lgtA) from N. meningitidis with SEQ ID NO:36 and the N-acetylglucosamine beta-1,4-galactosyltransferase (lgtB) from N. meningitidis with SEQ ID NO:38 to produce a mixture of oligosaccharides comprising 3′-SL, LN3, 3′-sialylated LN3 (Neu5Ac-a2,3-GlcNAc-b1,3-Gal-b1,4-Glc), LNnT and LSTd (Neu5Ac-a2,3-Gal-b1,4-GlcNAc-b1,3-Gal-b1,4-Glc). The novel strain is evaluated in a growth experiment on SD CSM-Trp-His drop-out medium comprising lactose as precursor according to the culture conditions provided in Example 3. After 72 h of incubation, the culture broth is harvested, and the sugars are analyzed on UPLC.

### Example 21. Production of 6′-Sialyllactose (6′-SL) with a Modified E. coli Strain

In a first step, an E. coli K-12 strain MG1655 is modified as described in Example 2 comprising knock-outs of the E. coli nagA, nagB, nanA, nanT, nanE, nanK, LacZ, LacY and LacA genes and with genomic knock-ins of constitutive transcriptional units containing the lactose permease (LacY) from E. coli with SEQ ID NO:22, the sialic acid transporter (nanT) from E. coli with SEQ ID NO:08, two copies of the L-glutamine D-fructose-6-phosphate aminotransferase (glmS*54) from E. coli with SEQ ID NO:06, two copies of the glucosamine 6-phosphate N-acetyltransferase (GNA1) from S. cerevisiae with SEQ ID NO:07, the N-acetylglucosamine 2-epimerase (AGE) from B. ovatus with SEQ ID NO:09, the N-acetylneuraminate synthase (NeuB) from N. meningitidis with SEQ ID NO:01, the acetyl-coenzyme A synthetase (acs) from E. coli with SEQ ID NO:47, the sucrose transporter (CscB) from E. coli W with SEQ ID NO:23, fructose kinase (Frk) from Z. mobilis with SEQ ID NO:24, the sucrose phosphorylase from B. adolescentis with SEQ ID NO:25 and two copies of the membrane transporter protein entS from E. coli with SEQ ID NO:49. The thus obtained mutant E. coli strain is further modified with genomic knock-ins and an expression plasmid with constitutive transcriptional units to express three N-acylneuraminate cytidylyltransferase isoproteins consisting of the NeuA enzyme from C. jejuni with SEQ ID NO:03, the NeuA enzyme from H. influenzae with SEQ ID NO:04 and the NeuA enzyme from P. multocida with SEQ ID NO:05, and three copies of the beta-galactoside alpha-2,6-sialyltransferase PdbST from P. damselae with SEQ ID NO:19. The novel strain is evaluated in a growth experiment according to the culture conditions provided in Example 2, in which the culture medium contains 30 g/L sucrose and 20 g/L lactose. The strain is grown in four biological replicates in a 96-well plate. After 72 h of incubation, the culture broth is harvested, and the sugars are analyzed on UPLC.

### Example 22. Production of 6′-Sialyllactose (6′-SL) with a Modified E. coli Strain

In a first step, an E. coli K-12 strain MG1655 is modified as described in Example 2 comprising knock-outs of the E. coli nagA, nagB, nanA, nanT, nanE, nanK, LacZ, LacY and LacA genes and with genomic knock-ins of constitutive transcriptional units containing the lactose permease (LacY) from E. coli with SEQ ID NO:22, the sialic acid transporter (nanT) from E. coli with SEQ ID NO:08, two copies of the L-glutamine D-fructose-6-phosphate aminotransferase (glmS*54) from E. coli with SEQ ID NO:06, two copies of the glucosamine 6-phosphate N-acetyltransferase (GNA1) from S. cerevisiae with SEQ ID NO:07, the N-acetylglucosamine 2-epimerase (AGE) from B. ovatus with SEQ ID NO:09, the N-acetylneuraminate synthase (NeuB) from N. meningitidis with SEQ ID NO:01, the acetyl-coenzyme A synthetase (acs) from E. coli with SEQ ID NO:47, the sucrose transporter (CscB) from E. col W with SEQ ID NO:23, fructose kinase (Frk) from Z. mobilis with SEQ ID NO:24, the sucrose phosphorylase from B. adolescentis with SEQ ID NO:25, the membrane transporter protein MdfA from E. coli with SEQ ID NO:50 and two copies of the membrane transporter protein entS from E. coli with SEQ ID NO:49. The thus obtained mutant E. coli strain is further modified with genomic knock-ins and an expression plasmid with constitutive transcriptional units to express three N-acylneuraminate cytidylyltransferase isoproteins consisting of the NeuA enzyme from C. jejuni with SEQ ID NO:03, the NeuA enzyme from H. influenzae with SEQ ID NO:04 and the NeuA enzyme from P. multocida with SEQ ID NO:05, and three copies of the beta-galactoside alpha-2,6-sialyltransferase PdbST from P. damselae with SEQ ID NO: 19. The novel strain is evaluated in a growth experiment according to the culture conditions provided in Example 2, in which the culture medium contains 30 g/L sucrose and 20 g/L lactose. The strain is grown in four biological replicates in a 96-well plate. After 72 h of incubation, the culture broth is harvested, and the sugars are analyzed on UPLC.

### Example 23. Production of 3′-Sialyllactose (3′-SL) with a Modified E. coli Strain

In a first step, an E. coli K-12 strain MG1655 is modified as described in Example 2 comprising knock-outs of the E. coli nagA, nagB, nanA, nanT, nanE, nanK, LacZ, LacY and LacA genes and with genomic knock-ins of constitutive transcriptional units containing the lactose permease (LacY) from E. coli with SEQ ID NO:22, the sialic acid transporter (nanT) from E. coli with SEQ ID NO:08, two copies of the L-glutamine D-fructose-6-phosphate aminotransferase (glmS*54) from E. coli with SEQ ID NO:06, two copies of the glucosamine 6-phosphate N-acetyltransferase (GNA1) from S. cerevisiae with SEQ ID NO:07, the N-acetylglucosamine 2-epimerase (AGE) from B. ovatus with SEQ ID NO:09, the N-acetylneuraminate synthase (NeuB) from N. meningitidis with SEQ ID NO:01, the acetyl-coenzyme A synthetase (acs) from E. coli with SEQ ID NO:47, the sucrose transporter (CscB) from E. coli W with SEQ ID NO:23, fructose kinase (Frk) from Z. mobilis with SEQ ID NO:24, the sucrose phosphorylase from B. adolescentis with SEQ ID NO:25, two copies of the membrane transporter protein MdfA from E. coli with SEQ ID NO:50 and two copies of the membrane transporter protein entS from E. coli with SEQ ID NO:49. The thus obtained mutant E. coli strain is further modified with genomic knock-ins and an expression plasmid with constitutive transcriptional units to express three N-acylneuraminate cytidylyltransferase isoproteins consisting of the NeuA enzyme from C. jejuni with SEQ ID NO:03, the NeuA enzyme from H. influenzae with SEQ ID NO:04 and the NeuA enzyme from P. multocida with SEQ ID NO:05, and three copies of the beta-galactoside alpha-2,3-sialyltransferase from P. multocida subsp. multocida str. Pm70 with SEQ ID NO:48. The novel strain is evaluated in a growth experiment according to the culture conditions provided in Example 2, in which the culture medium contains 30 g/L sucrose and 20 g/L lactose. The strain is grown in four biological replicates in a 96-well plate. After 72 h of incubation, the culture broth is harvested, and the sugars are analyzed on UPLC.

### Example 24. Material and Methods Bacillus subtilis

Media

Two different media are used, namely a rich Luria Broth (LB) and a minimal medium for shake flask (MMsf). The minimal medium uses a trace element mix.

Trace element mix consisted of 0.735 g/L CaCl₂)·2H₂O, 0.1 g/L MnCal₂·2H₂O, 0.033 g/L CuCl₂·2H₂O, 0.06 g/L COCl₂·6H₂O, 0.17 g/L ZnCl₂, 0.0311 g/L H₃BO₄, 0.4 g/L Na₂EDTA·2H₂O and 0.06 g/L Na₂MoO₄. The Fe-citrate solution contained 0.135 g/L FeCl₃·6H₂O, 1 g/L Na-citrate (Hoch 1973 PMC1212887).

The Luria Broth (LB) medium consisted of 1% tryptone peptone (Difco, Erembodegem, Belgium), 0.5% yeast extract (Difco) and 0.5% sodium chloride (VWR. Leuven, Belgium). Luria Broth agar (LBA) plates consisted of the LB media, with 12 g/L agar (Difco, Erembodegem, Belgium) added.

The minimal medium for the shake flasks (MMsf) experiments contained 2.00 g/L (NH₄)₂SO₄, 7.5 g/L KH₂PO₄, 17.5 g/L K₂HPO₄, 1.25 g/L Na-citrate, 0.25 g/L MgSO₄·7H₂O, 0.05 g/L tryptophan, from 10 up to 30 g/L glucose or another carbon source including but not limited to fructose, maltose, sucrose, glycerol and maltotriose when specified in the examples, 10 ml/L trace element mix and 10 ml/L Fe-citrate solution. The medium was set to a pH of 7 with 1M KOH. Depending on the experiment lactose, LNB or LacNAc could be added.

Complex medium, e.g., LB, was sterilized by autoclaving (121° C., 21′) and minimal medium by filtration (0.22 μm Sartorius). When necessary, the medium was made selective by adding an antibiotic (e.g., zeocin (20 mg/L)).

Strains, Plasmids and Mutations

Bacillus subtilis 168, available at Bacillus Genetic Stock Center (Ohio, USA).

Plasmids for gene deletion via Cre/lox are constructed as described by Yan et al. (Appl. & Environm. Microbial., September 2008, p5556-5562). Gene disruption is done via homologous recombination with linear DNA and transformation via electroporation as described by Xue et al. (J. Microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: Bba_K143012, Bba_K823000, Bba_K823002 or Bba_K823003. Cloning can be performed using Gibson Assembly, Golden Gate assembly, Cliva assembly, LCR or restriction ligation.

In an example for the production of lactose-based oligosaccharides, Bacillus subtilis mutant strains are created to contain a gene coding for a lactose importer (such as the E. coli lacY with SEQ ID NO:22). In an example for 2′FL, 3FL and/or diFL production, an alpha-1,2- and/or alpha-1,3-fucosyltransferase expression construct is additionally added to the strains. In an example for LN3 production, a constitutive transcriptional unit comprising a galactoside beta-1,3-N-acetylglucosaminyltransferase like e.g., lgtA from N. meningitidis (SEQ ID NO:236) is additionally added to the strain. In an example for LNT production, the LN3 producing strain is further modified with a constitutive transcriptional unit comprising an N-acetylglucosamine beta-1,3-galactosyltransferase like e.g., WbgO from E. coli 055:H₇ (SEQ ID NO:37). In an example for LNnT production, the LN3 producing strain is further modified with a constitutive transcriptional unit comprising an N-acetylglucosamine beta-1,4-galactosyltransferase like e.g., lgtB from N. meningitidis (SEQ ID NO:38).

In an example for sialic acid production, a mutant B. subtilis strain is created by overexpressing a fructose-6-P-aminotransferase like the native fructose-6-P-aminotransferase (UniProt ID P0CI73) to enhance the intracellular glucosamine-6-phosphate pool. Further on, the enzymatic activities of the genes nagA, nagB and gamA are disrupted by genetic knockouts and one or two copies of a glucosamine-6-P-aminotransferase like e.g., from S. cerevisiae (SEQ ID NO:07), an N-acetylglucosamine-2-epimerase like e.g., from B. ovatus (SEQ ID NO:09) and one or more N-acetylneuraminate synthases like e.g., from N. meningitidis (SEQ ID NO:01), C. jejuni (SEQ ID NO:02) are overexpressed on the genome. To allow sialylated oligosaccharide production, the sialic acid producing strain is further modified with a constitutive transcriptional unit comprising an N-acylneuraminate cytidylyltransferase like e.g., the NeuA enzyme from C. jejuni (SEQ ID NO:03), H. influenzae (SEQ ID NO:04) and P. multocida (SEQ ID NO:05), and one or more copies of a beta-galactoside alpha-2,3-sialyltransferase like e.g., PmultST3 from P. multocida (UniProt ID Q9CLP3) or a PmultST3-like polypeptide consisting of amino acid residues 1 to 268 of UniProt ID Q9CLP3 having beta-galactoside alpha-2,3-sialyltransferase activity (SEQ ID NO:17), or NmeniST3 from N. meningitidis (SEQ ID NO:18) or PmultST2 from P. multocida subsp. multocida str. Pm70 (GenBank No. AAK02592.1), a beta-galactoside alpha-2,6-sialyltransferase like e.g., PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity (SEQ ID NO:19) or P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity (SEQ ID NO:20), and/or an alpha-2,8-sialyltransferase like e.g., from M. musculus (SEQ ID NO:21).

Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL MMsf medium by diluting 400x. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 90° C. or for 60 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

### Example 25. Production of an Oligosaccharide Mixture Comprising 2′FL, 3-FL, DiFL, 3′SL, 6′SL, 3's-2′FL, 3's-3-FL, 6's-2′FL, 6's-3-FL with a Modified B. subtilis Host

A B. subtilis strain is modified as described in Example 24 by genomic knock-out of the nagA, nagB, glmS and gamA genes and genomic knock-ins of constitutive transcriptional units for the lactose permease (LacY) from E. coli with SEQ ID NO:22, the sucrose transporter (CscB) from E. coli W (SEQ ID NO:23), the fructose kinase (Frk) from Z. mobilis (SEQ ID NO:24), the sucrose phosphorylase (BaSP) from B. adolescentis (SEQ ID NO:25), the native fructose-6-P-aminotransferase (UniProt ID P0CI73), two copies of the glucosamine 6-phosphate N-acetyltransferase GNA1 from S. cerevisiae (SEQ ID NO:07), the mutant L-glutamine D-fructose-6-phosphate aminotransferase (glmS*54) from E. coli (SEQ ID NO:06), a phosphatase like e.g., a phosphatase chosen from the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from P. putida, ScDOG1 from S. cerevisiae or BsAraL from B. subtilis as described in WO 2018122225, the N-acetylglucosamine 2-epimerase (AGE) from B. ovatus (SEQ ID NO:09), the N-acetylneuraminate synthase (NeuB) from N. meningitidis (SEQ ID NO:01) and the N-acylneuraminate cytidylyltransferases NeuA from C. jejuni (SEQ ID NO:03), NeuA from H. influenzae (SEQ ID NO:04) and NeuA from P. multocida (SEQ ID NO:05). In a next step, the strain is transformed with an expression plasmid comprising constitutive transcriptional units for three copies of a PmultST3-like polypeptide consisting of amino acid residues 1 to 268 of UniProt ID Q9CLP3 having beta-galactoside alpha-2,3-sialyltransferase activity like SEQ ID NO:17 and three copies of a PdST6-like polypeptide consisting of amino acid residues 108 to 497 of UniProt ID 066375 having beta-galactoside alpha-2,6-sialyltransferase activity (SEQ ID NO:19). In a further step, the mutant strain is transformed with a second compatible expression plasmid comprising constitutive transcriptional units for the alpha-1,2-fucosyltransferase HpFutC with SEQ ID NO:26 and the alpha-1,3-fucosyltransferase HpFucT with SEQ ID NO:27. The novel strain is evaluated for the production of 2′FL, 3-FL, DiFL, 3′SL, 6′SL, 3'S-2′FL, 3'S-3-FL, 6'S-2′FL, 6'S-3-FL in a growth experiment on MMsf medium comprising lactose according to the culture conditions provided in Example 24. After 72 h of incubation, the culture broth is harvested, and the sugars are analyzed on UPLC.

### Example 26. Production of an Oligosaccharide Mixture Comprising 3′SL, LN3, LNT, Sialylated LN3 and LSTa with a Modified B. subtilis Host

A B. subtilis strain is modified for LN3 production and growth on sucrose as described in Example 24 by genomic knock-out of the nagA, nagB, glmS and gamA genes and genomic knock-ins of constitutive transcriptional units comprising genes encoding the lactose permease (LacY) from E. coli (SEQ ID NO:22), the native fructose-6-P-aminotransferase (UniProt ID P0CI73), the galactoside beta-1,3-N-acetylglucosaminyltransferase LgtA from N. meningitidis (SEQ ID NO:36), the sucrose transporter (CscB) from E. coli W (SEQ ID NO:23), the fructose kinase (Frk) from Z. mobilis (SEQ ID NO:24) and the sucrose phosphorylase (BaSP) from B. adolescentis (SEQ ID NO:25). In a next step, the mutant strain is further modified with a genomic knock-in of a constitutive transcriptional unit comprising the N-acetylglucosamine beta-1,3-galactosyltransferase WbgO from E. coli 055:H₇ (SEQ ID NO:37) to produce LNT. The mutant B. subtilis strain is further modified with genomic knock-ins of constitutive transcriptional units comprising two copies of the glucosamine 6-phosphate N-acetyltransferase GNA1 from S. cerevisiae (SEQ ID NO:07), two copies of the mutant L-glutamine D-fructose-6-phosphate aminotransferase (glmS*54) from E. coli (SEQ ID NO:06), a phosphatase like e.g., a phosphatase chosen from the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from P. putida, ScDOG1 from S. cerevisiae or BsAraL from B. subtilis as described in WO 2018122225, the N-acetylglucosamine 2-epimerase (AGE) from B. ovatus (SEQ ID NO:09), the N-acetylneuraminate synthase (NeuB) from N. meningitidis (SEQ ID NO:01), the N-acylneuraminate cytidylyltransferases NeuA from C. jejuni (SEQ ID NO:03), NeuA from H. influenzae (SEQ ID NO:04) and NeuA from P. multocida (SEQ ID NO:05) and three copies of a PmultST3-like polypeptide consisting of amino acid residues 1 to 268 of UniProt ID Q9CLP3 having beta-galactoside alpha-2,3-sialyltransferase activity like SEQ ID NO:17. The novel strain is evaluated for the production of a mixture comprising 3′SL, LN3, sialylated LN3, LNT, and LSTa (Neu5Ac-a2,3-Gal-b1,3-GlcNAc-b1,3-Gal-b1,4-Glc) in a growth experiment on MMsf medium comprising lactose as precursor according to the culture conditions provided in Example 24. After 72 h of incubation, the culture broth is harvested, and the sugars are analyzed on UPLC.

### Example 27. Material and Methods Corynebacterium glutamicum

Media

Two different media are used, namely a rich tryptone-yeast extract (TY) medium and a minimal medium for shake flask (MMsf). The minimal medium uses a 1000× stock trace element mix.

Trace element mix consisted of 10 g/L CaCl₂), 10 g/L FeSO₄·7H₂O, 10 g/L MnSO₄·H₂O, 1 g/L ZnSO₄·7H₂O, 0.2 g/L CuSO₄, 0.02 g/L NiCl₂·6H₂O, 0.2 g/L biotin (pH 7.0) and 0.03 g/L protocatechuic acid.

The minimal medium for the shake flasks (MMsf) experiments contained 20 g/L (NH₄)₂SO₄, 5 g/L urea, 1 g/L KH₂PO₄, 1 g/L K₂HPO₄, 0.25 g/L MgSO₄·7H₂O, 42 g/L MOPS, from 10 up to 30 g/L glucose or another carbon source including but not limited to fructose, maltose, sucrose, glycerol and maltotriose when specified in the examples and 1 ml/L trace element mix. Depending on the experiment lactose, LNB, and/or LacNAc could be added to the medium.

The TY medium consisted of 1.6% tryptone (Difco, Erembodegem, Belgium), 1% yeast extract (Difco) and 0.5% sodium chloride (VWR. Leuven, Belgium). TY agar (TYA) plates consisted of the TY media, with 12 g/L agar (Difco, Erembodegem, Belgium) added.

Complex medium, e.g., TY, was sterilized by autoclaving (121° C., 21′) and minimal medium by filtration (0.22 μm Sartorius). When necessary, the medium was made selective by adding an antibiotic (e.g., kanamycin, ampicillin).

Strains and Mutations

Corynebacterium glutamicum ATCC 13032, available at the American Type Culture Collection.

Integrative plasmid vectors based on the Cre/loxP technique as described by Suzuki et al. (Appl. Microbiol. Biotechnol., 2005 Apr., 67(2):225-33) and temperature-sensitive shuttle vectors as described by Okibe et al. (Journal of Microbiological Methods 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

In an example for the production of lactose-based oligosaccharides, C. glutamicum mutant strains are created to contain a gene coding for a lactose importer (such as e.g., the E. coli lacY with SEQ ID NO:22). In an example for 2′FL, 3FL and/or diFL production, an alpha-1,2- and/or alpha-1,3-fucosyltransferase expression construct is additionally added to the strains.

In an example for LN3 production, a constitutive transcriptional unit comprising a galactoside beta-1,3-N-acetylglucosaminyltransferase like e.g., lgtA from N. meningitidis (SEQ ID NO:36) is additionally added to the strain. In an example for LNT production, the LN3 producing strain is further modified with a constitutive transcriptional unit comprising an N-acetylglucosamine beta-1,3-galactosyltransferase like e.g., WbgO from E. coli 055:H₇ (SEQ ID NO:37). In an example for LNnT production, the LN3 producing strain is further modified with a constitutive transcriptional unit comprising an N-acetylglucosamine beta-1,4-galactosyltransferase like e.g., lgtB from N. meningitidis (SEQ ID NO:38).

In an example for sialic acid production, a mutant C. glutamicum strain is created by overexpressing a fructose-6-P-aminotransferase like the native fructose-6-P-aminotransferase (UniProt ID Q8NND3) to enhance the intracellular glucosamine-6-phosphate pool. Further on, the enzymatic activities of the genes nagA, nagB and gamA are disrupted by genetic knockouts and one or two copies of a glucosamine-6-P-aminotransferase like e.g., from S. cerevisiae (SEQ ID NO:07), an N-acetylglucosamine-2-epimerase like e.g., from B. ovatus (SEQ ID NO:09) and an N-acetylneuraminate synthase like e.g., from N. meningitidis (SEQ ID NO:01) are overexpressed on the genome. To allow sialylated oligosaccharide production, the sialic acid producing strain is further modified with a constitutive transcriptional unit comprising two or more N-acylneuraminate cytidylyltransferases like e.g., the NeuA from C. jejuni (SEQ ID NO:03), NeuA from H. influenzae (SEQ ID NO:04) and NeuA enzyme from P. multocida (SEQ ID NO:05), and one or more copies of a beta-galactoside alpha-2,3-sialyltransferase like e.g., PmultST3 from P. multocida (UniProt ID Q9CLP3) or a PmultST3-like polypeptide consisting of amino acid residues 1 to 268 of UniProt ID Q9CLP3 having beta-galactoside alpha-2,3-sialyltransferase activity (SEQ ID NO:17), or NmeniST3 from N. meningitidis (SEQ ID NO:18) or PmultST2 from P. multocida subsp. multocida str. Pm70 (GenBank No. AAK02592.1), a beta-galactoside alpha-2,6-sialyltransferase like e.g., PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity (SEQ ID NO:19) or P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity (SEQ ID NO:20), and/or an alpha-2,8-sialyltransferase like e.g., from M. musculus (SEQ ID NO:21).

Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

A preculture of 96-well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL MMsf medium by diluting 400x. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations, measured in the whole broth by the biomass, in relative percentages compared to the reference strain. The biomass is empirically determined to be approximately ⅓rd of the optical density measured at 600 nm.

### Example 28. Production of an Oligosaccharide Mixture Comprising LN3, Sialylated LN3, 6′SL, LNnT and LSTc with a Modified C. glutamicum Host

A C. glutamicum strain is modified as described in Example 27 for LN3 production and growth on sucrose by genomic knock-out of the ldh, cgl2645, nagB, gamA and nagA genes and genomic knock-ins of constitutive transcriptional units comprising genes encoding the lactose permease (LacY) from E. coli (SEQ ID NO:22), the native fructose-6-P-aminotransferase (UniProt ID Q8NND3), the galactoside beta-1,3-N-acetylglucosaminyltransferase LgtA from N. meningitidis (SEQ ID NO:36), the sucrose transporter (CscB) from E. coli W (SEQ ID NO:23), the fructose kinase (Frk) from Z. mobilis (SEQ ID NO:24) and the sucrose phosphorylase (BaSP) from B. adolescentis (SEQ ID NO:25). In a next step, the mutant strain is further modified with a genomic knock-in of a constitutive transcriptional unit comprising the N-acetylglucosamine beta-1,4-galactosyltransferase LgtB from N. meningitidis (SEQ ID NO:38) to produce LNnT. In a next step, the mutant strain is further modified with a genomic knock-in of a constitutive transcriptional unit comprising the native fructose-6-P-aminotransferase (UniProt ID Q8NND3), the glucosamine-6-P-aminotransferase from S. cerevisiae (SEQ ID NO:07), the N-acetylglucosamine-2-epimerase from B. ovatus (SEQ ID NO:09), and the N-acetylneuraminate synthase from N. meningitidis (SEQ ID NO:01) to produce sialic acid. In a next step, the novel strain is transformed with an expression plasmid comprising constitutive transcriptional units for the NeuA from C. jejuni (SEQ ID NO:03), NeuA from H. influenzae (SEQ ID NO:04) and NeuA enzyme from P. multocida (SEQ ID NO:05) and the beta-galactoside alpha-2,6-sialyltransferase PdST6 from P. damselae (UniProt ID 066375). The novel strain is evaluated for production of an oligosaccharide mixture comprising LN3, 6′-sialylated LN3 (Neu5Ac-a2,6-(GlcNAc-b1,3)-Gal-b1,4-Glc), 6′SL, LNnT and LSTc in a growth experiment on MMsf medium comprising lactose according to the culture conditions provided in Example 27. After 72 h of incubation, the culture broth is harvested, and the sugars are analyzed on UPLC.

### Example 29. Production of an Oligosaccharide Mixture Comprising 3′SL, 6′SL, LNB, 3′-Sialylated LNB and 6′-Sialylated LNB with a Modified C. glutamicum Host

A C. glutamicum strain is modified as described in Example 27 by genomic knock-out of the ldh, cgl2645, nagB, gamA and nagA genes and genomic knock-ins of constitutive transcriptional units comprising genes encoding the lactose permease (LacY) from E. coli (SEQ ID NO:22), WbgO with SEQ ID NO:37 from E. coli 055:H7, galE with SEQ ID NO:39 from E. coli, the native fructose-6-P-aminotransferase (UniProt ID Q8NND3), glmS*54 with SEQ ID NO:06, the glucosamine-6-P-aminotransferase from S. cerevisiae (SEQ ID NO:07), the N-acetylglucosamine-2-epimerase from B. ovatus (SEQ ID NO:09), and the N-acetylneuraminate synthase from N. meningitidis (SEQ ID NO:01). In a next step, the novel strain is transformed with an expression plasmid comprising constitutive transcriptional units for the NeuA enzyme from C. jejuni (SEQ ID NO:03), the NeuA enzyme from H. influenzae (SEQ ID NO:04) and the NeuA enzyme from P. multocida (SEQ ID NO:05), the beta-galactoside alpha-2,3-sialyltransferase PmultST3 from P. multocida (UniProt ID Q9CLP3) and the beta-galactoside alpha-2,6-sialyltransferase PdST6 from P. damselae (UniProt ID 066375). The novel strain is evaluated for production of an oligosaccharide mixture comprising 3′SL, 6′SL, LNB, 3′-sialylated LNB (3′SLNB) and 6′-sialylated LNB (6′SLNB) in a growth experiment on MMsf medium comprising lactose and glucose according to the culture conditions provided in Example 27. After 72 h of incubation, the culture broth is harvested, and the sugars are analyzed on UPLC.

### Example 30. Materials and Methods Chlamydomonas reinhardtii

Media

C. reinhardtii cells were cultured in Tris-acetate-phosphate (TAP) medium (pH 7.0). The TAP medium uses a 1000× stock Hutner's trace element mix. Hutner's trace element mix consisted of 50 g/L Na₂EDTA·H₂O (Titriplex III), 22 g/L ZnSO₄·7H₂O, 11.4 g/L H₃B03, 5 g/L MnCl₂·4H₂O, 5 g/L FeSO₄·7H₂O, 1.6 g/L COCl₂·6H₂O, 1.6 g/L CuSO₄·5H₂O and 1.1 g/L (NH₄)₆MoO₃.

The TAP medium contained 2.42 g/L Tris (tris(hydroxymethyl)aminomethane), 25 mg/L salt stock solution, 0.108 g/L K2HPO₄, 0.054 g/L KH₂PO₄ and 1.0 mL/L glacial acetic acid. The salt stock solution consisted of 15 g/L NH₄Cl, 4 g/L MgSO₄·7H₂O and 2 g/L CaCl₂)·2H₂O. As precursor(s) and/or acceptor(s) for saccharide synthesis, compounds like e.g., galactose, glucose, fructose, fucose, lactose, LacNAc, LNB could be added. Medium was sterilized by autoclaving (121° C., 21′). For stock cultures on agar slants TAP medium was used containing 1% agar (of purified high strength, 1000 g/cm²).

Strains, Plasmids and Mutations

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from Chlamydomonas Resource Center (www.chlamycollection.org), University of Minnesota, U.S.A.

Expression plasmids originated from pSI103, as available from Chlamydomonas Resource Center. Cloning can be performed using Gibson Assembly, Golden Gate assembly, Cliva assembly, LCR or restriction ligation. Suitable promoters for (heterologous) gene expression can be derived from e.g., Scranton et al. (Algal Res. 2016, 15: 135-142). Targeted gene modification (like gene knock-out or gene replacement) can be carried using the Crispr-Cas technology as described e.g., by Jiang et al. (Eukaryotic Cell 2014, 13(11): 1465-1469).

Transformation via electroporation was performed as described by Wang et al. (Biosci. Rep. 2019, 39: BSR2018210). Cells were grown in liquid TAP medium under constant aeration and continuous light with a light intensity of 8000 Lx until the cell density reached 1.0-2.0×10⁷ cells/mL. Then, the cells were inoculated into fresh liquid TAP medium in a concentration of 1.0×10⁶ cells/mL and grown under continuous light for 18-20 h until the cell density reached 4.0×10⁶ cells/mL. Next, cells were collected by centrifugation at 1250 g for 5 min at room temperature, washed and resuspended with pre-chilled liquid TAP medium containing 60 mM sorbitol (Sigma, U.S.A.), and iced for 10 min. Then, 250 μL of cell suspension (corresponding to 5.0×10⁷ cells) were placed into a pre-chilled 0.4 cm electroporation cuvette with 100 ng plasmid DNA (400 ng/mL). Electroporation was performed with 6 pulses of 500 V each having a pulse length of 4 ms and pulse interval time of 100 ms using a BTX ECM830 electroporation apparatus (1575Ω, 50 μFD). After electroporation, the cuvette was immediately placed on ice for 10 min. Finally, the cell suspension was transferred into a 50 ml conical centrifuge tube containing 10 mL of fresh liquid TAP medium with 60 mM sorbitol for overnight recovery at dim light by slowly shaking. After overnight recovery, cells were recollected and plated with starch embedding method onto selective 1.5% (w/v) agar-TAP plates containing ampicillin (100 mg/L) or chloramphenicol (100 mg/L). Plates were then incubated at 23+/−0.5° C. under continuous illumination with a light intensity of 8000 Lx. Cells were analyzed 5-7 days later.

In an example for production of UDP-galactose, C. reinhardtii cells are modified with transcriptional units comprising the genes encoding a galactokinase like e.g., from Arabidopsis thaliana (KIN, UniProt ID Q9SEE5) and an UDP-sugar pyrophosphorylase like e.g., USP from A. thaliana (UniProt ID Q9C5I1).

In an example for LN3 production, a constitutive transcriptional comprising a galactoside beta-1,3-N-acetylglucosaminyltransferase like e.g., lgtA from N. meningitidis (SEQ ID NO:36). In an example for LNT production, the LN3 producing strain is further modified with a constitutive transcriptional unit comprising an N-acetylglucosamine beta-1,3-galactosyltransferase like e.g., WbgO from E. coli 055:H7 (SEQ ID NO:37). In an example for LNnT production, the LN3 producing strain is further modified with a constitutive transcriptional unit comprising an N-acetylglucosamine beta-1,4-galactosyltransferase like e.g., lgtB from N. meningitidis (SEQ ID NO:38).

In an example for production of GDP-fucose, C. reinhardtii cells are modified with a transcriptional unit for a GDP-fucose synthase like e.g., from Arabidopsis thaliana (GER1, UniProt ID 049213).

In an example for fucosylation, C. reinhardtii cells can be modified with an expression plasmid comprising a constitutive transcriptional unit for an alpha-1,2-fucosyltransferase like e.g., HpFutC from H. pylori (SEQ ID NO:26) and/or an alpha-1,3-fucosyltransferase like e.g., HpFucT from H. pylori (SEQ ID NO:27).

In an example for CMP-sialic acid synthesis, C. reinhardtii cells are modified with constitutive transcriptional units for one or more UDP-N-acetylglucosamine-2-epimerase/N-acetylmannosamine kinases like e.g., GNE from Homo sapiens (UniProt ID Q9Y223) or a mutant form of the human GNE polypeptide comprising the R263L mutation or GNE from Mus musculus (UniProt ID Q91WG8), one or more N-acylneuraminate-9-phosphate synthetases like e.g., NANS from Homo sapiens (UniProt ID Q9NR45), NANS from Pseudomonas sp. UW4 (UniProt ID K9NPH9) and one or more N-acylneuraminate cytidylyltransferases like e.g., CMAS from Homo sapiens (UniProt ID Q8NFW8). In an example for production of sialylated oligosaccharides, C. reinhardtii cells are modified with a CMP-sialic acid transporter like e.g., CST from Mus musculus (UniProt ID Q61420), and a Golgi-localized sialyltransferase chosen from species like e.g., Homo sapiens, Mus musculus, Rattus norvegicus.

Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

Cells of C. reinhardtii were cultured in selective TAP-agar plates at 23+/−0.5° C. under 14/10 h light/dark cycles with a light intensity of 8000 Lx. Cells were analyzed after 5 to 7 days of cultivation.

For high-density cultures, cells could be cultivated in closed systems like e.g., vertical or horizontal tube photobioreactors, stirred tank photobioreactors or flat panel photobioreactors as described by Chen et al. (Bioresour. Technol. 2011, 102: 71-81) and Johnson et al. (Biotechnol. Prog. 2018, 34: 811-827).

### Example 31. Production of an Oligosaccharide Mixture Comprising Sialylated LNB and Sialylated LacNAc Structures in Mutant C. reinhardtii Cells

C. reinhardtii cells are engineered as described in Example 30 for production of CMP-sialic acid with genomic knock-ins of constitutive transcriptional units comprising GNE from Mus musculus (UniProt ID Q91WG8) and a mutant form of the human GNE (UniProt ID UniProt ID Q9Y223) differing from the native human GNE polypeptide with a R263L mutation, the N-acylneuraminate-9-phosphate synthetase NANS from Homo sapiens (UniProt ID Q9NR45) and the N-acylneuraminate cytidylyltransferase CMAS from Homo sapiens (UniProt ID Q8NFW8). In a next step, the cells are modified with genomic knock-ins of constitutive transcriptional units comprising the CMP-sialic acid transporter CST from Mus musculus (UniProt ID Q61420), the alpha-2,3-sialyltransferases (UniProt IDs P61943 and E9PSJ1) from Rattus norvegicus and the alpha-2,6-sialyltransferase (UniProt ID P13721) from Rattus norvegicus. In a final step, the cells are transformed with genomic knock-ins of constitutive transcriptional units comprising the Arabidopsis thaliana genes encoding the galactokinase (KIN, UniProt ID Q9SEE5) and the UDP-sugar pyrophosphorylase (USP) (UniProt ID Q9C₅I1), together with the N-acetylglucosamine beta-1,3-galactosyltransferase WbgO from E. coli 055:H₇ with SEQ ID NO:37 and the N-acetylglucosamine beta-1,4-galactosyltransferase LgtB from N. meningitidis with SEQ ID NO:38. The novel strain is evaluated for production of an oligosaccharide mixture comprising 3′-sialyllacto-N-biose (3′SLNB), 6′-sialyllacto-N-biose (6′SLNB), 3′-sialyllactosamine (3′SLacNAc) and 6′-sialyllactosamine (6′SLacNAc) in a cultivation experiment on TAP-agar plates comprising galactose, glucose and N-acetylglucosamine as precursors according to the culture conditions provided in Example 30. After 5 days of incubation, the cells are harvested, and the saccharide production is analyzed on UPLC.

### Example 32. Materials and Methods Animal Cells

Isolation of Mesenchymal Stem Cells from Adipose Tissue of Different Mammals

Fresh adipose tissue is obtained from slaughterhouses (e.g., cattle, pigs, sheep, chicken, ducks, catfish, snake, frogs) or liposuction (e.g., in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 370 C, 5% CO₂. The initial culture medium includes DMEM-F12, RPMI, and Alpha-MEM medium (supplemented with 15% fetal bovine serum), and 1% antibiotics. The culture medium is subsequently replaced with 10% FBS (fetal bovine serum)-supplemented media after the first passage. For example, Ahmad and Shakoori (2013, Stem Cell Regen Med. 9(2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% fetal bovine serum and 1% antibiotics under standard culture conditions. For example, Hassiotou et al. (2012, Stem Cells. 30(10): 2164-2174), which is incorporated herein by reference in its entirety for all purposes, describes certain variation(s) of the method(s) described herein in this example.

Differentiation of Stem Cells Using 2D and 3D Culture Systems

The isolated mesenchymal cells can be differentiated into mammary-like epithelial and luminal cells in 2D and 3D culture systems. See, for example, Huynh et al. 1991. Exp Cell Res. 197(2): 191-199; Gibson et al. 1991, In Vitro Cell Dev Biol Anim. 27(7): 585-594; Blatchford et al. 1999; Animal Cell Technology’: Basic & Applied Aspects, Springer, Dordrecht. 141-145; Williams et al. 2009, Breast Cancer Res 11(3): 26-43; and Arevalo et al. 2015, Am J Physiol Cell Physiol. 310(5): C348-C356; each of which is incorporated herein by reference in their entireties for all purposes.

For 2D culture, the isolated cells were initially seeded in culture plates in growth media supplemented with 10 ng/ml epithelial growth factor and 5 pg/ml insulin. At confluence, cells were fed with growth medium supplemented with 2% fetal bovine serum, 1% penicillin-streptomycin (100 U/ml penicillin, 100 ug/ml streptomycin), and 5 pg/ml insulin for 48h. To induce differentiation, the cells were fed with complete growth medium containing 5 pg/ml insulin, 1 pg/ml hydrocortisone, 0.65 ng/ml triiodothyronine, 100 nM dexamethasone, and 1 pg/ml prolactin. After 24h, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/ml epithelial growth factor and 5 pg/ml insulin. At confluence, cells were fed with growth medium supplemented with 2% fetal bovine serum, 1% penicillin-streptomycin (100 U/ml penicillin, 100 ug/ml streptomycin), and 5 pg/ml insulin for 48 h. To induce differentiation, the cells were fed with complete growth medium containing 5 pg/ml insulin, 1 pg/ml hydrocortisone, 0.65 ng/ml triiodothyronine, 100 nM dexamethasone, and 1 pg/ml prolactin. After 24 h, serum is removed from the complete induction medium.

Method of Making Mammary-Like Cells

Mammalian cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodelling is performed using remodelling systems such as CRISPR/Cas9, to activate select genes of interest, such as casein, a-lactalbumin to be constitutively on, to allow for the expression of their respective proteins, and/or to down-regulate and/or knock-out select endogenous genes as described e.g., in WO 2021067641, which is incorporated herein by reference in its entirety for all purposes.

Cultivation

Completed growth media includes high glucose DMEM/F12, 10% FBS, 1% NEAA, 1% pen/strep, 1% ITS-X, 1% F-Glu, 10 ng/ml EGF, and 5 pg/ml hydrocortisone. Completed lactation media includes high glucose DMEM/F12, 1% NEAA, 1% pen/strep, 1% ITS-X, 1% F-Glu, 10 ng/ml EGF, 5 pg/ml hydrocortisone, and 1 pg/ml prolactin (5 ug/ml in Hyunh 1991). Cells are seeded at a density of 20,000 cells/cm² onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example, the use of nickel resins to remove His-tagged growth factors, to further reduce the levels of contaminants in the lactated product.

### Example 33. Evaluation of LacNAc, Sialylated LacNAc Structures and Sialyl-Lewis x Production in a Non-Mammary Adult Stem Cell

Isolated mesenchymal cells and re-programmed into mammary-like cells as described in Example 32 are modified via CRISPR-CAS to over-express the beta-1,4-galactosyltransferase 4 B4GalT4 from Homo sapiens (UniProt ID 060513), the GDP-fucose synthase GFUS from Homo sapiens (UniProt ID Q13630), the galactoside alpha-1,3-fucosyltransferase FUT3 from Homo sapiens (UniProt ID P21217), the N-acylneuraminate cytidylyltransferases from Mus musculus (UniProt ID Q99KK2) and from Homo sapiens (UniProt ID Q8NFW8), and the CMP-N-acetylneuraminate-beta-1,4-galactoside alpha-2,3-sialyltransferase ST3GAL3 from Homo sapiens (UniProt ID Q11203) and the alpha-2,6-sialyltransferase (UniProt ID P13721) from Rattus norvegicus. All genes introduced in the cells are codon-optimized to the host. Cells are seeded at a density of 20,000 cells/cm² onto collagen coated flasks in completed growth media and left to adhere and expand for 48 hours in completed growth media, after which the media is switched out for completed lactation media for about 7 days. After cultivation as described in Example 32, cells are subjected to UPLC to analyze for production of LacNAc, 3′-sialylated LacNAc, 6′-sialylated LacNAc and sialyl-Lewis x.

1-51. (canceled)

52. A cell that is metabolically engineered for production of a sialylated disaccharide and/or sialylated oligosaccharide, the cell comprising a pathway for production of the sialylated disaccharide and/or sialylated oligosaccharide,
wherein the cell is modified for expression and/or overexpression of multiple coding DNA sequences encoding one or more proteins that catalyze a same chemical reaction.

53. The cell of claim 52, wherein at least one of the proteins is involved in the pathway for production of the sialylated disaccharide and/or sialylated oligosaccharide.

54. The cell of claim 52, wherein the pathway for production of sialylated disaccharide and/or sialylated oligosaccharide comprises a sialylation pathway comprising at least one protein selected from the group consisting of N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-aceylneuraminate-9-phosphate synthetase, phosphatase, N-acetylneuraminate synthase, N-acylneuraminate cytidylyltransferase, sialyltransferase, and sialic acid transporter.

55. The cell of claim 52, wherein the multiple coding DNA sequences comprise one or more of
multiple copies of the same coding DNA sequence encoding one protein,
multiple coding DNA sequences encoding one protein, and
multiple coding DNA sequences encoding multiple isoproteins that catalyze a same chemical reaction.

56. The cell of claim 52, wherein multiple is at least two (2).

57. The cell of claim 52, wherein the coding DNA sequences are presented to the cell in one or more gene expression modules wherein expression is regulated by one or more regulatory sequences; the coding DNA sequences are presented to the cell in one or more gene expression modules wherein the expression modules are integrated in the cell's genome; or the coding DNA sequences are presented to the cell in one or more gene expression modules wherein the expression modules are presented on a vector comprising a plasmid, cosmid, phage, liposome, or virus, which is to be stably transformed in the cell.

58. The cell of claim 52, wherein at least one of the proteins is involved in the synthesis of a nucleotide-activated sugar, wherein the nucleotide-activated sugar is to be used in the production of the sialylated disaccharide and/or sialylated oligosaccharide, and the nucleotide-activated sugar is selected from the group consisting of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac2, CMP-Neu5,9Ac2, CMP-Neu5,7(8,9)Ac2, CMP-N-glycolylneuraminic acid (CMP-Neu5Gc), GDP-fucose (GDP-Fuc), GDP-rhamnose, and UDP-xylose.

59. The cell of claim 58, wherein the protein involved in the synthesis of a nucleotide-activated sugar is selected from the group consisting of mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, N-acetylmannosamine-6-phosphate 2-epimerase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, CMP-sialic acid synthetase, galactose-1-epimerase, galactokinase, glucokinase, galactose-1-phosphate uridylyltransferase, UDP-glucose 4-epimerase, glucose-1-phosphate uridylyltransferase, glucophosphomutase, N-acetylglucosamine 1-phosphate uridylyltransferase, UDP-N-acetylglucosamine 4-epimerase, UDP-galactose 4-epimerase, N-acetylgalactosamine kinase, UDP-GalNAc pyrophosphorylase, mannose-1-phosphate guanyltransferase, UDP-GlcNAc 2-epimerase, and ManNAc kinase.

60. The cell of claim 52, wherein the cell further expresses at least one glycosyltransferase selected from the group consisting of a fucosyltransferase, sialyltransferase, galactosyltransferase, glucosyltransferase, mannosyltransferase, N-acetylglucosaminyltransferase, N-acetylgalactosaminyltransferase, N-acetylmannosaminyltransferase, xylosyltransferase, glucuronyltransferase, galacturonyltransferase, glucosaminyltransferase, N-glycolylneuraminyltransferase, rhamnosyltransferase, N-acetylrhamnosyltransferase, UDP-4-amino-4,6-dideoxy-N-acetyl-beta-L-altrosamine transaminase, UDP-N-acetylglucosamine enolpyruvyl transferase, and fucosaminyltransferase.

61. The cell of claim 52, wherein at least one of the proteins is a membrane transporter protein that is selected from the group consisting of a siderophore exporter, an ATP-binding cassette (ABC) transporter, a major facilitator superfamily (MFS) transporter, and a sugar efflux transporter.

62. The cell of claim 52, wherein the sialylated disaccharide and/or sialylated oligosaccharide is selected from the group consisting of a milk oligosaccharide, O-antigen, enterobacterial common antigen (ECA), oligosaccharide repeats present in capsular polysaccharides, peptidoglycan, amino-sugars, and Lewis-type antigen oligosaccharides.

63. The cell of claim 52, wherein the cell comprises a fucosylation pathway comprising at least one protein selected from the group consisting of a mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, and fucosyltransferase.

64. The cell of claim 52, wherein the cell comprises a galactosylation pathway comprising at least one protein selected from the group consisting of a galactose-1-epimerase, galactokinase, glucokinase, galactose-1-phosphate uridylyltransferase, UDP-glucose 4-epimerase, glucose-1-phosphate uridylyltransferase, glucophosphomutase, and galactosyltransferase.

65. The cell of claim 52, wherein the cell comprises an N-acetylglucosaminylation pathway comprising at least one protein selected from the group consisting of a L-glutamine-D-fructose-6-phosphate aminotransferase, N-acetylglucosamine-6-phosphate deacetylase, phosphoglucosamine mutase, N-acetylglucosamine-1-phosphate uridylyltransferase/glucosamine-1-phosphate acetyltransferase, and N-acetylglucosaminyltransferase.

66. The cell of claim 52, wherein the cell is modified for enhanced synthesis or supply of phosphoenolpyruvate (PEP).

67. The cell of claim 52, wherein the cell comprises:
at least one coding DNA sequence encoding a protein selected from the group consisting of i) an enzyme from Neisseria meningitidis (NmNeuB) comprising SEQ ID NO:01 and having N-acetylneuraminate synthase activity, ii) a functional homolog or functional fragment of the enzyme comprising SEQ ID NO:01, and iii) a polypeptide sequence having at least 80% sequence identity to the full-length sequence of the enzyme comprising SEQ ID NO:01 and having N-acetylneuraminate synthase activity,
two or more coding DNA sequences encoding a protein selected from the group consisting of i) an enzyme from Campylobacter jejuni (CjNeuA) comprising SEQ ID NO:02, Helicobacter influenzae (HiNeuA) comprising SEQ ID NO:03, and Pasteurella multocida (PmultNeuA) comprising SEQ ID NO:04, wherein the enzymes comprising SEQ ID NOs:02, 03, and 04 have N-acylneuraminate cytidylyltransferase activity, ii) a functional homolog or functional fragment of the enzymes comprising SEQ ID NOs:02, 03, or 04, and iii) a polypeptide sequence having at least 80% sequence identity to the full-length sequence of the enzymes comprising SEQ ID NOs:02, 03, or 04, respectively, and having N-acylneuraminate cytidylyltransferase activity, and
two or more copies of one or more coding DNA sequences of an alpha-2,3-sialyltransferase, an alpha-2,6-sialyltransferase, or an alpha-2,8-sialyltransferase.

68. The cell of claim 52, wherein the cell comprises:
two or more copies of a coding DNA sequence encoding an enzyme having L-glutamine-D-fructose-6-phosphate aminotransferase activity and being selected from the group consisting of i) an enzyme from Escherichia coli (glmS*54) comprising SEQ ID NO:05, ii) a functional homolog or functional fragment of the enzyme comprising SEQ ID NO:05, and iii) a polypeptide sequence having at least 80% sequence identity to the full-length sequence of the enzyme comprising SEQ ID NO:05 and having L-glutamine-D-fructose-6-phosphate aminotransferase activity, or
two or more copies of a coding DNA sequence encoding an enzyme having glucosamine 6-phosphate N-acetyltransferase activity and being selected from the group consisting of i) an enzyme from Saccharomyces cerevisiae (GNA1) comprising SEQ ID NO:06, ii) a functional homolog or functional fragment of the enzyme comprising SEQ ID NO:06, and iii) a polypeptide sequence having at least 80% sequence identity to the full-length sequence of the enzyme comprising SEQ ID NO:06 and having glucosamine 6-phosphate N-acetyltransferase activity.

69. The cell of claim 52, wherein the cell comprises a modification for reduced production of acetate.

70. The cell of claim 52, wherein the cell further comprises a lower or reduced expression, or abolished, impaired, reduced or delayed activity of one or more of the proteins comprising beta-galactosidase, galactoside 0-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, or pyruvate decarboxylase.

71. The cell of claim 52, wherein the cell comprises a catabolic pathway for selected monosaccharides, disaccharides or oligosaccharides which is at least partially inactivated, the monosaccharides, disaccharides, or oligosaccharides being involved in or required for the synthesis of the sialylated disaccharide and/or sialylated oligosaccharide.

72. The cell of claim 52, wherein the cell uses a precursor for synthesizing the sialylated disaccharide and/or sialylated oligosaccharide, the precursor being fed to the cell from a cultivation medium or produced by the cell.

73. The cell of claim 52, wherein the cell produces a precursor for synthesizing the sialylated disaccharide and/or sialylated oligosaccharide.

74. The cell of claim 52, wherein the cell produces 90 g/L or more of the sialylated disaccharide or sialylated oligosaccharide in whole broth or supernatant, wherein the sialylated disaccharide or sialylated oligosaccharide in the whole broth or supernatant has a purity of at least 80% measured on the total amount of sialylated disaccharide or sialylated oligosaccharide and its precursor produced by the cell in the whole broth or supernatant, respectively.

75. The cell of claim 52, wherein the cell is a bacterium, a fungus, a yeast, a plant cell, an animal cell, or a protozoan cell.

76. The cell of claim 75, wherein the cell is a viable Gram-negative bacterium that comprises a reduced or abolished synthesis of poly-N-acetyl-glucosamine (PNAG), enterobacterial common antigen (ECA), cellulose, colanic acid, core oligosaccharides, osmoregulated periplasmic glucans (OPG), glucosylglycerol, glycan, or trehalose compared to a non-modified progenitor.

77. The cell of claim 52, wherein the cell is capable of synthesizing a mixture of oligosaccharides comprising at least one sialylated oligosaccharide.

78. The cell of claim 52, wherein the cell is capable of synthesizing a mixture of disaccharides and oligosaccharides comprising at least one sialylated disaccharide or at least one sialylated oligosaccharide.

79. A method for producing a sialylated disaccharide and/or sialylated oligosaccharide by a cell, the method comprising:
i) cultivating the cell of claim 52 in a culture medium under conditions permissive to produce the sialylated disaccharide and/or sialylated oligosaccharide, and
ii) optionally separating the sialylated disaccharide and/or sialylated oligosaccharide from the cultivation.

80. The method according to claim 79, wherein the culture medium comprises a carbon source comprising a monosaccharide, disaccharide, oligosaccharide, polysaccharide, polyol, glycerol, a complex medium including molasses, corn steep liquor, peptone, tryptone or yeast extract.

81. The method according to claim 79, wherein the cell uses at least one precursor for synthesizing the sialylated disaccharide and/or sialylated oligosaccharide.

82. The method according to claim 79, wherein the culture medium contains at least one compound selected from the group consisting of lactose, galactose, sialic acid, fucose, GlcNAc, GalNAc, lacto-N-biose (LNB), and N-acetyllactosamine (LacNAc).

83. The method according to claim 79, wherein a first phase of exponential cell growth is provided by adding a carbon-based substrate to the culture medium before lactose is added to the culture medium in a second phase.

84. The method according to claim 79 wherein the cell produces at least one precursor for synthesizing the sialylated disaccharide and/or sialylated oligosaccharide.

85. The method according to claim 81, wherein the precursor for synthesizing the sialylated disaccharide and/or sialylated oligosaccharide is completely converted into the synthesizing the sialylated disaccharide and/or sialylated oligosaccharide.

86. The method according to claim 79, wherein the sialylated disaccharide or sialylated oligosaccharide is separated from the culture medium or the cell, wherein the separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis, affinity chromatography, ion exchange chromatography, hydrophobic interaction chromatography, gel filtration, or ligand exchange chromatography.

87. The method according to claim 79, wherein the method further comprises purifying the sialylated disaccharide and/or sialylated oligosaccharide, optionally the purification comprises at least one of the following steps: use of activated charcoal or carbon, use of charcoal, nanofiltration, ultrafiltration, electrophoresis, enzymatic treatment or ion exchange, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying, or vacuum roller drying.

88. A method of producing a mixture of oligosaccharides comprising at least one sialylated oligosaccharide by a cell, the method comprising the steps of:
i) cultivating a cell of claim 77 in culture medium under conditions permissive to produce the mixture of oligosaccharides, and
ii) separating the mixture of oligosaccharides from the cultivation.

89. A method of producing a mixture of disaccharides and oligosaccharides comprising at least one sialylated disaccharide or at least one sialylated oligosaccharide by a cell, the method comprising the steps of:
i) cultivating the cell of claim 78 in culture medium under conditions permissive to produce the mixture of disaccharides and oligosaccharides, and
ii) optionally separating the mixture of disaccharides and oligosaccharides from the cultivation.


SEQUENCE LISTING (57 sequences)

SEQ ID NO: 1 - protein, 349 aa, Neisseria meningitidis
MQNNNEFKIGNRSVGYNHEPLIICEIGINHEGSLKTAFEMVDAAYNAGAEVVKHQTHIVEDEMSDEAKQVIPGNADVSIYEIMERCALNEEDEIKLKEYVESKGMIFISTPFSRAAALRLQRMDIPAYKIGSGECNNYPLIKLVASFGKPIILSTGMNSIESIKKSVEIIREAGVPYALLHCTNIYPTPYEDVRLGGMNDLSEAFPDAIIGLSDHTLDNYACLGAVALGGSILERHFTDRMDRPGPDIVCSMNPDTFKELKQGAHALKLARGGKKDTIIAGEKPTKDFAFASVVADKDIKKGELLSGDNLWVKRPGNGDFSVNEYETLFGKVAACNIRKGAQIKKTDIE

SEQ ID NO: 2 - protein, 346 aa, Campylobacter jejuni
MKEIKIQNIIISEEKAPLVVPEIGINHNGSLELAKIMVDAAFSAGAKIIKHQTHIVEDEMSKAAKKVIPGNAKISIYEIMQKCALDYKDELALKEYTEKLGLVYLSTPFSRAGANRLEDMGVSAFKIGSGECNNYPLIKHIAAFKKPMIVSTGMNSIESIKPTVKILLDNEIPFVLMHTTNLYPTPHNLVRLNAMLELKKEFSCMVGLSDHTTDNLACLGAVVLGACVLERHFTDSMHRSGPDIVCSMDTKALKELIIQSEQMAIIRGNNESKKAAKQEQVTIDFAFASVVSIKDIKKGEVLSMDNIWVKRPGLGGISAAEFENILGKKALRDIENDAQLSYEDFA

SEQ ID NO: 3 - protein, 221 aa, Campylobacter jejuni
MSLAIIPARGGSKGIKNKNLVLLNNKPLIYYTIKAALNAKSISKVVVSSDSDEILNYAKSQNVDILKRPISLAQDDTTSDKVLLHALKFYKDYEDVVFLQPTSPLRTNIHINEAFNLYKNSNANALISVSECDNKILKAFVCNDCGDLAGICNDEYPFMPRQKLPKTYMSNGAIYILKIKEFLNNPSFLQSKTKHFLMDESSSLDIDCLEDLKKVEQIWKK

SEQ ID NO: 4 - protein, 224 aa, Haemophilus influenzae
MTRIAIIPARAGSKGIKDKNLQLVGGVSLVGRAILAAQESGMFDQIVVTSDGENILKEATKYGAKAVARPESLAQSDTRTIDAILHCLETLNISQGTAALLQPTSPLRNALDIRNAMEIFLGGKYKSVVSACECEHHPYKSFTLEGTEVQPIHELTDFEAPRQKLPKSYRANGAIYTNDIQSLFEEKRFFIAPMRFYLMPTYRSIDIDSTLDLQLAESLISKEF

SEQ ID NO: 5 - protein, 223 aa, Pasteurella multocida
MTNIAIIPARAGSKGIPDKNLQPVGGHSLIGRAILAAKNADVFDMIVVTSDGDNILREAEKYGALALKRPAELAQDNSRTIDAILHALESLNIREGTCTLLQPTSPLRDHLDIKNAMDMYVNGGVHSVVSACECEHHPYKAFALSKDHEVLPVREIADFEAARQTLPKMYRANGAIYINDIAQLLKEKYFFIPPLKFYLMPTYRSVDIDVKQDLELAEILSNK

SEQ ID NO: 6 - protein, 609 aa, Escherichia coli
MCGIVGAIAQRDVAEILLEGLRRLEYRGYDSAGLAVVDTEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYCHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLSRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 7 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 8 - protein, 496 aa, Escherichia coli
MSTTTQNIPWYRHLNRAQWRAFSAAWLGYLLDGFDFVLIALVLTEVQGEFGLTTVQAASLISAAFISRWFGGLMLGAMGDRYGRRLAMVTSIVLFSAGTLACGFAPGYITMFIARLVIGMGMAGEYGSSATYVIESWPKHLRNKASGFLISGFSVGAVVAAQVYSLVVPVWGWRALFFIGILPIIFALWLRKNIPEAEDWKEKHAGKAPVRTMVDILYRGEHRIANIVMTLAAATALWFCFAGNLQNAAIVAVLGLLCAAIFISFMVQSAGKRWPTGVMLMVVVLFAFLYSWPIQALLPTYLKTDLAYNPHTVANVLFFSGFGAAVGCCVGGFLGDWLGTRKAYVCSLLASQLLIIPVFAIGGANVWVLGLLLFFQQMLGQGIAGILPKLIGGYFDTDQRAAGLGFTYNVGALGGALAPIIGALIAQRLDLGTALASLSFSLTFVVILLIGLDMPSRVQRWLRPEALRTHDAIDGKPFSGAVPFGSAKNDLVKTKS

SEQ ID NO: 9 - protein, 421 aa, Bacteroides ovatus
MDSKNNIGHSADISLTAELPIPIYNGNTIMDFKKLASLYKDELLDNVLPFWLEHSQDHEYGGYFTCLDREGKVFDTDKFIWLQSREVWMFSMLYNKVEKRQEWLDCAIQGGEFLKKYGHDGNYNWYFSLDRSGRPLVEPYNIFSYTFATMAFGQLSLTTGNQEYADIAKKTFDIILSKVDNPKGRWNKLHPGTRNLKNFALPMILCNLALEIEHLLDETYLRETMDTCIHEVMEVFYRPELGGIIVENVDIDGNLVDCFEGRQVTPGHAIEAMWFIMDLGKRLNRPELIEKAKETTLTMLNYGWDKQYGGIYYFMDRNGCPPQQLEWDQKLWWVHIETLISLLKGYQLTGDKKCLEWFEKVHDYTWEHFKDKEYPEWYGYLNRRGEVLLPLKGGKWKGCFHVPRGLYQCWKTLEEIKNIVS

SEQ ID NO: 10 - protein, 445 aa, Escherichia coli
MSNRKYFGTDGIRGRVGDAPITPDFVLKLGWAAGKVLARHGSRKIIIGKDTRISGYMLESALEAGLAAAGLSALFTGPMPTPAVAYLTRTFRAEAGIVISASHNPFYDNGIKFFSIDGTKLPDAVEEAIEAEMEKEISCVDSAELGKASRIVDAAGRYIEFCKATFPNELSLSELKIVVDCANGATYHIAPNVLRELGANVIAIGCEPNGVNINAEVGATDVRALQARVLAEKADLGIAFDGDGDRVIMVDHEGNKVDGDQIMYIIAREGLRQGQLRGGAVGTLMSNMGLELALKQLGIPFARAKVGDRYVLEKMQEKGWRIGAENSGHVILLDKTTTGDGIVAGLQVLAAMARNHMSLHDLCSGMKMFPQILVNVRYTAGSGDPLEHESVKAVTAEVEAALGNRGRVLLRKSGTEPLIRVMVEGEDEAQVTEFAHRIADAVKAV

SEQ ID NO: 11 - protein, 456 aa, Escherichia coli
MLNNAMSVVILAAGKGTRMYSDLPKVLHTLAGKAMVQHVIDAANELGAAHVHLVYGHGGDLLKQALKDDNLNWVLQAEQLGTGHAMQQAAPFFADDEDILMLYGDVPLISVETLQRLRDAKPQGGIGLLTVKLDDPTGYGRITRENGKVTGIVEHKDATDEQRQIQEINTGILIANGADMKRWLAKLTNNNAQGEYYITDIIALAYQEGREIVAVHPQRLSEVEGVNNRLQLSRLERVYQSEQAEKLLLAGVMLRDPARFDLRGTLTHGRDVEIDTNVIIEGNVTLGHRVKIGTGCVIKNSVIGDDCEISPYTVVEDANLAAACTIGPFARLRPGAELLEGAHVGNFVEMKKARLGKGSKAGHLTYLGDAEIGDNVNIGAGTITCNYDGANKFKTIIGDDVFVGSDTQLVAPVTVGKGATIAAGTTVTRNVGENALAISRVPQTQKEGWRRPVKKK

SEQ ID NO: 12 - protein, 373 aa, Campylobacter jejuni
MVKKILFITGSRADYSKIKSLMYRVQNSSEFELYIFATGMHLSKNFGYTVKELYKNGFKNIYEFINYDKYYQTDKALATTIDGFSRYANELKPDLIVVHGDRIEPLAAAIVGALNNILVAHIEGGEISGTIDDSLRHAISKLAHIHLVNDEFAKRRLMQLGEDEKSIFIIGSPDLELLNDNKISLSEAKKYYDINYENYALLMFHPVTTEITSIKNQADNLVKALIQSNKNYIVIYPNNDLGFELILQSYEEFKNNPRFKLFPSLRFEYFITLLKNADFIIGNSSCILKEALYLKTAGILVGSRQNGRLGNENTLKVNANSDEILKAINTIHKKQDLFSAKLEILDSSKLFFEYLQSGDFFKLSTQKVFKDIK

SEQ ID NO: 13 - protein, 722 aa, Mus musculus
MEKNGNNRKLRVCVATCNRADYSKLAPIMFGIKTEPAFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKCKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERISLILQMCVEAAAEAVKLNCRILGVGISTGGRVNPQEGVVLHSTKLIQEWNSVDLRTPLSDTLHLPVWVDNDGNCAAMAERKFGQGKGQENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNVKAQSILRTAGTALGLGVVNILHTMNPSLVILSGVLASHYIHIVKDVIRQQALSSVQDVDVVVSDLVDPALLGAASMVLDYTTRRIH

SEQ ID NO: 14 - protein, 348 aa, Syntrophorhabdus sp.
MESFSFGKSTVSKNGPCYIIAEIGNNHQGDLKIALKMIKTAAGMGVNAVKFQKRDNRTLYTKSMYNKPYENENSYGSTYGEHRDYLEFCWDKYVEMKKCAEDNDVEFMCTAFDFKSVDFLEQLGITSYKVASGDVTNTPLLEYIAQTKKPMFVSTGGATLEEIRVAYDTVTKYNDRVCLLHCVAGYPTEYEYLNLNTINILKKEFPDTLIGYSGHDYGILAPVVAYMLGAVVVEKHFTLNRAWKGTDHKFSLEPTGLYKMTRDLRRVDVSIGNGKKVVHAFEKDPISKMSKSLYASRKLQAGRVLTADDISIKSPGGGIPPYKMEEVLGKMLKINLPEEALFSFDYFE

SEQ ID NO: 15 - protein, 241 aa, Candidatus Magnetomorum sp.
MPSLKAIFFDLDNTLVDCSTADIRTYEILASIARDKVRGINSPALINHFQKQLINVPFDPEKQIPVHPWRTILWTKALKSENIDDIDLAEKLNIAFHRERLAFYTFIYGSQKMLKKLLKLYTCVIITNGDSEIQRPKLKACKAQKYFSHIIVGGEEPYEKPHPSIFKKACEMAKCKPESAIIIGDSLETDIKGGLDAGLASTIWINPKNKPIPEKGPVPHYQAVSVLELPLIIQHIQQQQQ

SEQ ID NO: 16 - protein, 164 aa, Bacteroides thetaiotaomicron
MKEIKLILTDIDGVWTDGGMFYDQTGNEWKKFNTSDSAGIFWAHNKGIPVGILTGEKTEIVRRRAEKLKVDYLFQGVVDKLSAAEELCNELGINLEQVAYIGDDLNDAKLLKRVGIAGVPASAPFYIRRLSTIFLEKRGGEGVFREFVEKVLGINLEDFIAVIQ

SEQ ID NO: 17 - protein, 268 aa, Pasteurella multocida
MDKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLPPHFVYEKLG

SEQ ID NO: 18 - protein, 371 aa, Neisseria meningitidis
MGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 19 - protein, 391 aa, Photobacterium damselae
MTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 20 - protein, 498 aa, Photobacterium sp.
MSEENTQSIIKNDINKTIIDEEYVNLEPINQSNISFTKHSWVQTCGTQQLLTEQNKESISLSVVAPRLDDDEKYCFDFNGVSNKGEKYITKVTLNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSAEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWAGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCIAV

SEQ ID NO: 21 - protein, 380 aa, Mus musculus
MRNCKMARVASVLGLVMLSVALLILSLISYVSLKKENIFTTPKYASPGAPRMYMFHAGFRSQFALKFLDQSFVPITNSLTHELQEKPSKWTFNRTAFLHQRQEILQHVDVIKNFSLTKSSVRIGQLMHYDYSSHKYVFSISNNFRSLLPDVSPIMNKRYNVCAVVGNSGILTGSQCGQEIDKSDFVFRCNFAPTEAFHKDVGRKTNLTTFNPSILEKYYNNLLTIQDRNNFFLSLKKLDGAILWIPAFFFHTSATVTRTLVDFFVEHRGQLKVQLAWPGNIMQHVNRYWKNKHLSPKRLSTGILMYTLASAICEEIHLYGFWPFGFDPNTREDLPYHYYDKKGTKFTTKWQESHQLPAEFQLLYRMHGEGLTKLTLSHCA

SEQ ID NO: 22 - protein, 417 aa, Escherichia coli [sequence omitted]

SEQ ID NO: 23 - protein, 415 aa, Escherichia coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 24 - protein, 301 aa, Zymomonas mobilis
MKNDKKIYGCIEGGGTKFMLALIDSDRKMLAVERVPTTTPEETLGKSVEFFKKALPQYADSFASFGIASFGPLCLDRKSPKWGYITNTPKPFWPNTDVVTPFKEAFGCPVEIDTDVNGAALAENFWGASKGTHTSVYVTVGTGFGGGVLIDGKPIHGLAHPEMGHGIPIRHPDDRDFEGCCPYHGGCYEGLASGTAIRKRWGKALNEMEPAEFEKAREIIAFYLAHFNVTLQAFISPERIVFGGGVMHVDGMLASVRRQTAEIANSYFEGADFEKIIVLPGLGDQAGMMGAFALALAAENK

SEQ ID NO: 25 - protein, 504 aa, Bifidobacterium adolescentis [sequence omitted]

SEQ ID NO: 26 - protein, 299 aa, Helicobacter pylori
MAFKVVQICGGLGNQMFQYAFAKSLQKHSNTPVLLDITSFDWSNRKMQLELFPIDLPYASEKEIAIAKMQHLPKLVRNVLKCMGFDRVSQEIVFEYEPKLLKTSRLTYFYGYFQDPRYFDAISPLIKQTFTLPPPPENGNNKKKEEEYHRKLALILAAKNSVFVHIRRGDYVGIGCQLGIDYQKKALEYMAKRVPNMELFVFCEDLEFTQNLDLGYPFMDMTTRDKEEEAYWDMLLMQSCKHGIIANSTYSWWAAYLINNPEKIIIGPKHWLFGHENILCKEWVKIESHFEVKSQKYNA

SEQ ID NO: 27 - protein, 478 aa, Helicobacter pylori
MFQPLLDAYVESASIEKMASKSPPPLKIAVANWWGDEEIKEFKNSVLYFILSQRYTITLHQNPNEFSDLVFGNPLGSARKILSYQNAKRVFYTGENESPNFNLFDYAIGFDELDFNDRYLRMPLYYDRLHHKAESVNDTTAPYKLKDNSLYALKKPSHCFKEKHPNLCAVVNDESDPLKRGFASFVASNPNAPIRNAFYDALNSIEPVTGGGSVRNTLGYNVKNKNEFLSQYKFNLCFENTQGYGYVTEKIIDAYFSHTIPIYWGSPSVAKDFNPKSFVNVHDFKNFDEAIDYIKYLHTHKNAYLDMLYENPLNTLDGKAYFYQNLSFKKILAFFKTILENDTIYHDNPFIFCRDLNEPLVTIDDLRVNYDDLRVNYDDLRINYDDLRVNYDDLRINYDDLRVNYDDLRVNYDDLRINYDDLRVNYDDLRVNYERLLSKATPLLELSQNTTSKIYRKAYQKSLPLLRAIRRWVKKLGL

SEQ ID NO: 28 - protein, 264 aa, Escherichia coli
MKQYLELMQKVLDEGTQKNDRTGTGTLSIFGHQMRFNLQDGFPLVTTKRCHLRSIIHELLWFLQGDTNIAYLHENNVTIWDEWADENGDLGPVYGKQWRAWPTPDGRHIDQITTVLNQLKNDPDSRRIIVSAWNVGELDKMALAPCHAFFQFYVADGKLSCQLYQRSCDVFLGLPFNIASYALLVHMMAQQCDLEVGDFVWTGGDTHLYSNHMDQTHLQLSREPRPLPKLIIKRKPESIFDYRFEDFEIEGYDPHPGIKAPVAI

SEQ ID NO: 29 - protein, 391 aa, Escherichia coli
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 30 - protein, 456 aa, Escherichia coli
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 31 - protein, 478 aa, Escherichia coli
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 32 - protein, 373 aa, Escherichia coli [sequence omitted]

SEQ ID NO: 33 - protein, 321 aa, Escherichia coli
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 34 - protein, 438 aa, Escherichia coli
MGNTSIQTQSYRAVDKDAGQSRSYIIPFALLCSLFFLWAVANNLNDILLPQFQQAFTLTNFQAGLIQSAFYFGYFIIPIPAGILMKKLSYKAGIITGLFLYALGAALFWPAAEIMNYTLFLVGLFIIAAGLGCLETAANPFVTVLGPESSGHFRLNLAQTFNSFGAIIAVVFGQSLILSNVPHQSQDVLDKMSPEQLSAYKHSLVLSVQTPYMIIVAIVLLVALLIMLTKFPALQSDNHSDAKQGSFSASLSRLARIRHWRWAVLAQFCYVGAQTACWSYLIRYAVEEIPGMTAGFAANYLTGTMVCFFIGRFTGTWLISRFAPHKVLAAYALIAMALCLISAFAGGHVGLIALTLCSAFMSIQYPTIFSLGIKNLGQDTKYGSSFIVMTIIGGGIVTPVMGFVSDAAGNIPTAELIPALCFAVIFIFARFRSQTATN

SEQ ID NO: 35 - protein, 949 aa, Bacteroides fragilis
MQKLLSLPSNLVQSFHELERVNRTDWFCTSDPVGKKLGSGGGTSWLLEECYNEYSDGATFGEWLEKEKRILLHAGGQSRRLPGYAPSGKILTPVPVFRWERGQHLGQNLLSLQLPLYEKIMSLAPDKLHTLIASGDVYIRSEKPLQSIPEADVVCYGLWVDPSLATHHGVFASDRKHPEQLDFMLQKPSLAELESLSKTHLFLMDIGIWLLSDRAVEILMKRSHKESSEELKYYDLYSDFGLALGTHPRIEDEEVNTLSVAILPLPGGEFYHYGTSKELISSTLSVQNKVYDQRRIMHRKVKPNPAMFVQNAVVRIPLCAENADLWIENSHIGPKWKIASRHIITGVPENDWSLAVPAGVCVDVVPMGDKGFVARPYGLDDVFKGDLRDSKTTLTGIPFGEWMSKRGLSYTDLKGRTDDLQAVSVFPMVNSVEELGLVLRWMLSEPELEEGKNIWLRSEHFSADEISAGANLKRLYAQREEFRKGNWKALAVNHEKSVFYQLDLADAAEDFVRLGLDMPELLPEDALQMSRIHNRMLRARILKLDGKDYRPEEQAAFDLLRDGLLDGISNRKSTPKLDVYSDQIVWGRSPVRIDMAGGWTDTPPYSLYSGGNVVNLAIELNGQPPLQVYVKPCKDFHIVLRSIDMGAMEIVSTFDELQDYKKIGSPFSIPKAALSLAGFAPAFSAVSYASLEEQLKDFGAGIEVTLLAAIPAGSGLGTSSILASTVLGAINDFCGLAWDKNEICQRTLVLEQLLTTGGGWQDQYGGVLQGVKLLQTEAGFAQSPLVRWLPDHLFTHPEYKDCHLLYYTGITRTAKGILAEIVSSMFLNSSLHLNLLSEMKAHALDMNEAIQRGSFVEFGRLVGKTWEQNKALDSGTNPPAVEAIIDLIKDYTLGYKLPGAGGGGYLYMVAKDPQAAVRIRKILTENAPNPRARFVEMTLSDKGFQVSRS

SEQ ID NO: 36 - protein, 348 aa, Neisseria meningitidis
MPSEAFRRHRAYRENKLQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGVWLDFAANGRMRRLFTLRQYFGILHRLLKNR

SEQ ID NO: 37 - protein, 265 aa, Escherichia coli
MIIDEAESAESTHPVVSVILPVNKKNPFLDEAINSILSQTFSSFEIIIVANCCTDDFYNELKHKVNDKIKLIRTNIAYLPYSLNKAIDLSNGEFIARMDSDDISHPDRFTKQVDFLKNNPYVDVVGTNAIFIDDKGREINKTKLPEENLDIVKNLPYKCCIVHPSVMFRKKVIASIGGYMFSNYSEDYELWNRLSLAKIKFQNLPEYLFYYRLHEGQSTAKKNLYMVMVNDLVIKMKCFFLTGNINYLFGGIRTIASFIYCKYIK

SEQ ID NO: 38 - protein, 275 aa, Neisseria meningitidis
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 39 - protein, 338 aa, Escherichia coli
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 40 - protein, 587 aa, Kluyveromyces lactis
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 41 - protein, 792 aa, Escherichia coli
MSNNGSSPLVLWYNQLGMNDVDRVGGKNASLGEMITNLSGMGVSVPNGFATTADAFNQFLDQSGVNQRIYELLDKTDIDDVTQLAKAGAQIRQWIIDTPFQPELENAIREAYAQLSADDENASFAVRSSATAEDMPDASFAGQQETFLNVQGFDAVLVAVKHVFASLFNDRAISYRVHQGYDHRGVALSAGVQRMVRSDLASSGVMFSIDTESGFDQVVFITSAWGLGEMVVQGAVNPDEFYVHKPTLAANRPAIVRRTMGSKKIRMVYAPTQEHGKQVKIEDVPQEQRDIFSLTNEEVQELAKQAVQIEKHYGRPMDIEWAKDGHTGKLFIVQARPETVRSRGQVMERYTLHSQGKIIAEGRAIGHRIGAGPVKVIHDISEMNRIEPGDVLVTDMTDPDWEPIMKKASAIVTNRGGRTCHAAIIARELGIPAVVGCGDATERMKDGENVTVSCAEGDTGYVYAELLEFSVKSSSVETMPDLPLKVMMNVGNPDRAFDFACLPNEGVGLARLEFIINRMIGVHPRALLEFDDQEPQLQNEIREMMKGFDSPREFYVGRLTEGIATLGAAFYPKRVIVRLSDFKSNEYANLVGGERYEPDEENPMLGFRGAGRYVSDSFRDCFALECEAVKRVRNDMGLTNVEIMIPFVRTVDQAKAVVEELARQGLKRGENGLKIIMMCEIPSNALLAEQFLEYFDGFSIGSNDMTQLALGLDRDSGVVSELFDERNDAVKALLSMAIRAAKKQGKYVGICGQGPSDHEDFAAWLMEEGIDSLSLNPDTVVQTWLSLAELKK

SEQ ID NO: 42 - protein, 610 aa, Corynebacterium glutamicum
MTTAAIRGLQGEAPTKNKELLNWIADAVELFQPEAVVFVDGSQAEWDRMAEDLVEAGTLIKLNEEKRPNSYLARSNPSDVARVESRTFICSEKEEDAGPTNNWAPPQAMKDEMSKHYAGSMKGRTMYVVPFCMGPISDPDPKLGVQLTDSEYVVMSMRIMTRMGIEALDKIGANGSFVKCLHSVGAPLEPGQEDVAWPCNDTKYITQFPETKEIWSYGSGYGGNAILAKKCYALRIASVMAREEGWMAEHMLILKLINPEGKAYHIAAAFPSACGKTNLAMITPTIPGWTAQVVGDDIAWLKLREDGLYAVNPENGFFGVAPGTNYASNPIAMKTMEPGNTLFTNVALTDDGDIWWEGMDGDAPAHLIDWKGNDWTPESDENAAHPNSRYCVAIDQSPAAAPEFNDWEGVKIDAILFGGRRADTVPLVTQTYDWEHGTMVGALLASGQTAASAEAKVGTLRHDPMAMLPFIGYNAGEYLQNWIDMGNKGGDKMPSIFLVNWFRRGEDGRFLWPGFGDNSRVLKWVIDRIEGRVGADETVVGHTAKAEDLDLDGLDTPIEDVKEALTAPAEQWANDVQDNAEYLTFLGPRVPAEVHSQFDALKARISAAHA

SEQ ID NO: 43 - protein, 540 aa, Escherichia coli
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTRQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 44 - protein, 213 aa, Escherichia coli
MKNWKTSAESILTTGPVVPVIVVKKLEHAVPMAKALVAGGVRVLEVTLRTECAVDAIRAIAKEVPEAIVGAGTVLNPQQLAEVTEAGAQFAISPGLTEPLLKAATEGTIPLIPGISTVSELMLGMDYGLKEFKFFPAEANGGVKALQAIAGPFSQVRFCPTGGISPANYRDYLALKSVLCIGGSWLVPADALEAGDYDRITKLAREAVEGAKL

SEQ ID NO: 45 - protein, 565 aa, Escherichia coli
MEPKTKKQRSLYIPYAGPVLLEFPLLNKGSAFSMEERRNFNLLGLLPEVVETIEEQAERAWIQYQGFKTEIDKHIYLRNIQDTNETLFYRLVNNHLDEMMPVIYTPTVGAACERFSEIYRRSRGVFISYQNRHNMDDILQNVPNHNIKVIVVTDGERILGLGDQGIGGMGIPIGKLSLYTACGGISPAYTLPVVLDVGTNNQQLLNDPLYMGWRNPRITDDEYYEFVDEFIQAVKQRWPDVLLQFEDFAQKNAMPLLNRYRNEICSFNDDIQGTAAVTVGTLIAASRAAGGQLSEKKIVFLGAGSAGCGIAEMIISQTQREGLSEEAARQKVFMVDRFGLLTDKMPNLLPFQTKLVQKRENLSDWDTDSDVLSLLDVVRNVKPDILIGVSGQTGLFTEEIIREMHKHCPRPIVMPLSNPTSRVEATPQDIIAWTEGNALVATGSPFNPVVWKDKIYPIAQCNNAFIFPGIGLGVIASGASRITDEMLMSASETLAQYSPLVLNGEGMVLPELKDIQKVSRAIAFAVGKMAQQQGVAVKTSAEALQQAIDDNFWQAEYRDYRRTSI

SEQ ID NO: 46 - protein, 759 aa, Escherichia coli
MDDQLKQSALDFHEFPVPGKIQVSPTKPLATQRDLALAYSPGVAAPCLEIEKDPLKAYKYTARGNLVAVISNGTAVLGLGNIGALAGKPVMEGKGVLFKKFAGIDVFDIEVDELDPDKFIEVVAALEPTFGGINLEDIKAPECFYIEQKLRERMNIPVFHDDQHGTAIISTAAILNGLRVVEKNISDVRMVVSGAGAAAIACMNLLVALGLQKHNIVVCDSKGVIYQGREPNMAETKAAYAVVDDGKRTLDDVIEGADIFLGCSGPKVLTQEMVKKMARAPMILALANPEPEILPPLAKEVRPDAIICTGRSDYPNQVNNVLCFPFIFRGALDVGATAINEEMKLAAVRAIAELAHAEQSEVVASAYGDQDLSFGPEYIIPKPFDPRLIVKIAPAVAKAAMESGVATRPIADFDVYIDKLTEFVYKTNLFMKPIFSQARKAPKRVVLPEGEEARVLHATQELVTLGLAKPILIGRPNVIEMRIQKLGLQIKAGVDFEIVNNESDPRFKEYWTEYFQIMKRRGVTQEQAQRALISNPTVIGAIMVQRGEADAMICGTVGDYHEHFSVVKNVFGYRDGVHTAGAMNALLLPSGNTFIADTYVNDEPDAEELAEITLMAAETVRRFGIEPRVALLSHSNFGSSDCPSSSKMRQALELVRERAPELMIDGEMHGDAALVEAIRNDRMPDSSLKGSANILVMPNMEAARISYNLLRVSSSEGVTVGPVLMGVAKPVHVLTPIASVRRIVNMVALAVVEAQTQPL

SEQ ID NO: 47 - protein, 652 aa, Escherichia coli
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 48 - protein, 308 aa, Pasteurella multocida
MNLIICCTPLQVLIAEKIIAKFPHMPFYGVMLSTVSNKKFDFYAKRLAQQCQGFFSMVQHKDRFNLLKEILYLKRTFSGKHFDQVFVANINDLQIQFLLSAIDFNLLNTFDDGTINIVPNSLFYQDDPATLQRKLINVLLGNKYSIQSLRALSHTHYTIYKGFKNIIERVEPIELVAADNSEKVTSAVINVLLGQPVFAEDERNIALAERVIKQFNIHYYLPHPREKYRLAQVNYIDTELIFEDYILQQCQTHKYCVYTYFSSAIINIMNKSDNIEVVALKIDTENPAYDACYDLFDELGVNVIDIRE

SEQ ID NO: 49 - protein, 416 aa, Escherichia coli
MNKQSWLLNLSLLKTHPAFRAVFLARFISIVSLGLLGVAVPVQIQMMTHSTWQVGLSVTLTGGAMFVGLMVGGVLADRYERKKVILLARGTCGIGFIGLCLNALLPEPSLLAIYLLGLWDGFFASLGVTALLAATPALVGRENLMQAGAITMLTVRLGSVISPMIGGLLLATGGVAWNYGLAAAGTFITLLPLLSLPALPPPPQPREHPLKSLLAGFRFLLASPLVGGIALLGGLLTMASAVRVLYPALADNWQMSAAQIGFLYAAIPLGAAIGALTSGKLAHSARPGLLMLLSTLGSFLAIGLFGLMPMWILGVVCLALFGWLSAVSSLLQYTMLQTQTPEAMLGRINGLWTAQNVTGDAIGAALLGGLGAMMTPVASASASGFGLLIIGVLLLLVLVELRHFRQTPPQVTASDS

SEQ ID NO: 50 - protein, 410 aa, Escherichia coli
MQNKLASGARLGRQALLFPLCLVLYEFSTYIGNDMIQPGMLAVVEQYQAGIDWVPTSMTAYLAGGMFLQWLLGPLSDRIGRRPVMLAGVVWFIVTCLAILLAQNIEQFTLLRFLQGISLCFIGAVGYAAIQESFEEAVCIKITALMANVALIAPLLGPLVGAAWIHVLPWEGMFVLFAALAAISFFGLQRAMPETATRIGEKLSLKELGRDYKLVLKNGRFVAGALALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLVNGILWLSLMVIFLKDKQMGNSHEG

SEQ ID NO: 51 - protein, 471 aa, Escherichia coli
MTDLPDSTRWQLWIVAFGFFMQSLDTTIVNTALPSMAQSLGESPLHMHMVIVSYVLTVAVMLPASGWLADKVGVRNIFFTAIVLFTLGSLFCALSGTLNELLLARALQGVGGAMMVPVGRLTVMKIVPREQYMAAMTFVTLPGQVGPLLGPALGGLLVEYASWHWIFLINIPVGIIGAIATLLLMPNYTMQTRRFDLSGFLLLAVGMAVLTLALDGSKGTGLSPLTIAGLVAVGVVALVLYLLHARNNNRALFSLKLFRTRTFSLGLAGSFAGRIGSGMLPFMTPVFLQIGLGFSPFHAGLMMIPMVLGSMGMKRIVVQVVNRFGYRRVLVATTLGLSLVTLLFMTTALLGWYYVLPFVLFLQGMVNSTRFSSMNTLTLKDLPDNLASSGNSLLSMIMQLSMSIGVTIAGLLLGLFGSQHVSVDSGTTQTVFMYTWLSMALIIALPAFIFARVPNDTHQNVAISRRKRSAQ

SEQ ID NO: 52 - protein, 334 aa, Escherichia coli
MNAVTEGRKVLLEIADLKVHFEIKDGKQWFWQPPKTLKAVDGVTLRLYEGETLGVVGESGCGKSTFARAIIGLVKATDGHVAWLGKELLGMKPDEWRAVRSDIQMIFQDPLASLNPRMTIGEIIAEPLRTYHPKMSRQEVRERVKAMMLKVGLLPNLINRYPHEFSGGQCQRIGIARALILEPKLIICDEPVSALDVSIQAQVVNLLQQLQREMGLSLIFIAHDLAVVKHISDRVLVMYLGHAVELGTYDEVYHNPLHPYTRALMSAVPIPDPDLEKNKTIQLLEGELPSPINPPSGCVFRTRCPIAGPECAKTRPVLEGSFRHSVSCLKVDPL

SEQ ID NO: 53 - protein, 584 aa, Lactococcus lactis
MANRIEGKAVDKTSIKHFIKLIRAAKPRYLFFIIGILAGIVGTLIQLQVPKMVQPLVNSFGHGVNGGKVALVIALYIGSAAVSAIAAIVLGIFGESVVKNLRTRVWDKMIHLPVKYFDEVKTGEMSSRLANDTTQVKNLIANSIPQAFTSILLLVGSIVFMLQMQWRLTLAMIIAVPVVMLIMFPIMTFGQKIGRTRQDSLANFQGIASESLSEIRLVKSSNAEKQASKKAENDVNALYKIGVKEAIFDGLMSPVMMLSMMLMIFGLLAYGIYLISTGVMSLGTLLGMMMYLMNLIGAVPTVATFFTELAKASGSTGRLTELLDEEQEVLHQGESLDLEGKTLSARHVDFAYDDSEQILRDISFEAQPNSIIAFAGPSGGGKSTIFSLLERFYQPTAGEITIDGQPIDNISLENWRSQIGFVSQDSAIMAGTIRENLTYGLEGDYTDEDLWQVLDLAFARSFVENMPDQLNTEVGERGVKISGGQRQRLAIARAFLRNPKILMLDEATASLDSESESMVQKALDSLMKGRTTLVIAHRLSTIVDADKIYFIEKGQITGSGKHNELVATHPLYAKYVSEQLTVGQ

SEQ ID NO: 54 - protein, 375 aa, Bifidobacterium longum
MAEVVFDHVTRIYPGNDKPSVDDLNLDIKDGEFLVLVGPSGCGKSTTLRMLAGLEEVNKGRILIGGKDVTTMQPKDRDIAMVFQNYALYPHMTVADNMGFALKIAGTPKDEIRKRVEKAAEILDLTEYLDRKPKALSGGQRQRVAMGRAIVREPKVFLMDEPLSNLDAKLRVQTRTQIAALQRQLGVTTLYVTHDQTEALTMGDRIAVIKLGILQQVGAPTELYDRPANVFVAGFIGSPSMNLNTHPVVNGKAKIGEDTVDLPAEAVNKLTAEDNGQIVVGFRPEDAGLAPVDDPNAFSLKVVNVEDLGSDGYIYGTIVTDGSAAEASQVMSDQNKLTTIRVNPRALPKVGATVKIKIDPAKMHLFAPSTELRLN

SEQ ID NO: 55 - protein, 392 aa, Escherichia coli
MIWIMTMARRMNGVYAAFMLVAFMMGVAGALQAPTLSLFLSREVGAQPFWIGLFYTVNAIAGIGVSLWLAKRSDSQGDRRKLIIFCCLMAIGNALLFAFNRHYLTLITCGVLLASLANTAMPQLFALAREYADNSAREVVMFSSVMRAQLSLAWVIGPPLAFMLALNYGFTVMFSIAAGIFTLSLVLIAFMLPSVARVELPSENALSMQGGWQDSNVRMLFVASTLMWTCNTMYIIDMPLWISSELGLPDKLAGFLMGTAAGLEIPAMILAGYYVKRYGKRRMMVIAVAAGVLFYTGLIFFNSRMALMTLQLFNAVFIGIVAGIGMLWFQDLMPGRAGAATTLFTNSISTGVILAGVIQGAIAQSWGHFAVYWVIAVISVVALFLTAKVKDV

SEQ ID NO: 56 - protein, 393 aa, Escherichia coli
MHNSPAVSSAKSFDLTSTAFLIVAFLTGIAGALQTPTLSIFLTDEVHARPAMVGFFFTGSAVIGILVSQFLAGRSDKRGDRKSLIVFCCLLGVLACTLFAWNRNYFVLLFVGVFLSSFGSTANPQMFALAREHADKTGREAVMFSSFLRAQVSLAWVIGPPLAYALAMGFSFTVMYLSAAVAFIVCGVMVWLFLPSMRKELPLATGTIEAPRRNRRDTLLLFVICTLMWGSNSLYIINMPLFIINELHLPEKLAGVMMGTAAGLEIPTMLIAGYFAKRLGKRFLMRVAAVGGVCFYAGMLMAHSPVILLGLQLLNAIFIGILGGIGMLYFQDLMPGQAGSATTLYTNTSRVGWIIAGSVAGIVAEIWNYHAVFWFAMVMIIATLFCLLRIKDV

SEQ ID NO: 57 - protein, 394 aa, Escherichia coli
MQKTATTPSKILDLTAAAFLLVAFLTGIAGALQTPTLSIFLADELKARPIMVGFFFTGSAIMGILVSQFLARHSDKQGDRKLLILLCCLFGVLACTLFAWNRNYFILLSTGVLLSSFASTANPQMFALAREHADRTGRETVMFSTFLRAQISLAWVIGPPLAYELAMGFSFKVMYLTAAIAFVVCGLIVWLFLPSIQRNIPVVTQPVEILPSTHRKRDTRLLFVVCSMMWAANNLYMINMPLFIIDELHLTDKLTGEMIGIAAGLEIPMMLIAGYYMKRIGKRLLMLIAIVSGMCFYASVLMATTPAVELELQILNAIFLGILCGIGMLYFQDLMPEKIGSATTLYANTSRVGWIIAGSVDGIMVEIWSYHALFWLAIGMLGIAMICLLFIKDI